(19) United States
(12) Patent Application Publication
(10) Pub. No.: US 2004/0254136 A1
(43) Pub. Date: Dec. 16, 2004

(54) Antisense oligonucleotides and related methods for regulating cell death

(76) Inventors: Carol M. Troy, Hastings-on-Hudson, NY (US); Michael L. Shelanski, Brooklyn, NY (US)

Correspondence Address: John P White, Cooper & Dunham, 1185 Avenue of the America, New York, NY, 10036, US

(21) Appl. No.: 10/482,952

(22) PCT Filed: Jul. 3, 2002
(86) PCT No.: PCT/US02/21002

(30) Foreign Application Priority Data
Jul. 3, 2001 (US) 09/898,158

(51) Int. Cl. A61K 48/00; C12Q 1/68; C07H 21/04

(52) U.S. Cl. 514/44; 435/6; 536/23.2

(57) ABSTRACT

This invention provides a first nucleic acid which specifically hybridizes to a nucleic acid encoding an inhibitor-of-apoptosis protein. This invention also provides related compositions and methods for inducing cell death and treating cancer using same. This invention further provides a second nucleic acid which specifically hybridizes to a nucleic acid encoding a protein, other than caspase-2, that induces cell death. Finally, this invention provides related compositions and methods for inhibiting cell death, inhibiting neuronal cell death in particular, and treating a neurodegenerative disorder and a heart disorder using the second nucleic acid.

This application is a continuation-in-part and claims priority of U.S. Ser. No. 09/898,158, filed Jul. 3, 2001, the contents of which are hereby incorporated by reference into this application.

The invention disclosed herein was made with government support under Grant Nos. N535933 and N515076 from the National Institutes of Health. Accordingly, the United States Government has certain rights in this invention.

### BACKGROUND OF THE INVENTION

Throughout this application various references are cited by author and date. Full bibliographic citations for these references may be found at the end of the Experimental Details section. Disclosures of these publications in their entireties are hereby incorporated by reference into this application to more fully describe the state of the art to which this invention pertains.

During development, neurons that fail to find appropriate targets and sources of target-derived neurotrophic factors undergo apoptotic cell death (Pettmann and Henderson 1998). The elimination of improperly connected neurons constitutes a critical step in the formation of specific connections in the nervous system.

Caspases are an evolutionarily conserved family of proteins with at least 14 mammalian members (Thornberry and Lazebnik 1998). Data from caspase-null and Apafl-null mice support a role for the caspase-9 pathway in mediating death occurring early in the development of the nervous system (Kuida et al. 1996; Hakem et al. 1998; Kuida et al. 1998; Yoshida et al. 1998), when mainly neuroblasts are being removed. It is not clear which caspases are necessary for the removal of neurons which occurs later in development. It has been reported that the caspase-dependency of a pathway can be altered in caspase-null mice (Zheng et al. 2000) but no mechanism was provided for the observations described. Studies of the caspases required to execute trophic factor deprivation (TFD)-induced death of NGF-dependent neurons have given apparently conflicting results. In the case of cultured rat and mouse sympathetic neurons, as well as of neuronal PC12 cells, there is evidence that caspase-2 is necessary for TFD-induced death. Both acute downregulation of caspase-2 expression with an antisense oligonucleotide (Troy et al. 1997) and chronic downregulation of caspase-2 in PC12 cells by stable transfection with antisense caspase-2 (Haviv et al. 1998) protect these cells from NGF deprivation. In contrast, sympathetic neurons cultured from caspase-2-null mice retain sensitivity to NGF-deprivation and die (Bergeron et al. 1998). Other studies have demonstrated a delay in TFD-induced death in sympathetic neurons from caspase-9-null embryos (Deshmukh et al. 2000).

An understanding of the mechanism of neuronal TFD-induced death which would reconcile these apparently contradictory data has never been achieved.

### SUMMARY OF THE INVENTION

This invention provides a first nucleic acid which specifically hybridizes to a nucleic acid encoding an inhibitor-of-apoptosis protein.

This invention also provides a composition comprising the first nucleic acid and a carrier.

This invention further provides a method for inducing a cell's death which comprises contacting the cell with the first nucleic acid under conditions permitting the nucleic acid to enter the cell.

This invention further provides a method for treating a subject afflicted with cancer which comprises administering to the subject a therapeutically effective amount of the first nucleic acid.

This invention provides a second nucleic acid that specifically hybridizes to a nucleic acid which encodes a protein, other than caspase-2, that induces cell death.

This invention further comprises a composition comprising the second nucleic acid and a carrier.

This invention further provides method for inhibiting a cell's death which comprises contacting the cell with the second nucleic acid under conditions permitting the nucleic acid to enter the cell.

This invention further provides a method for inhibiting a neuronal cell's death which comprises contacting the cell with the second nucleic acid under conditions permitting the nucleic acid to enter the cell.

This invention further provides a method for treating a neurodegenerative disorder in a subject which comprises administering to the subject a therapeutically effective amount of the second nucleic acid.

Finally, this invention provides a method for treating a heart disorder in a subject which comprises administering to the subject a therapeutically effective amount of the second nucleic acid.

### BRIEF DESCRIPTION OF THE FIGURES

FIGS. 1A and 1B

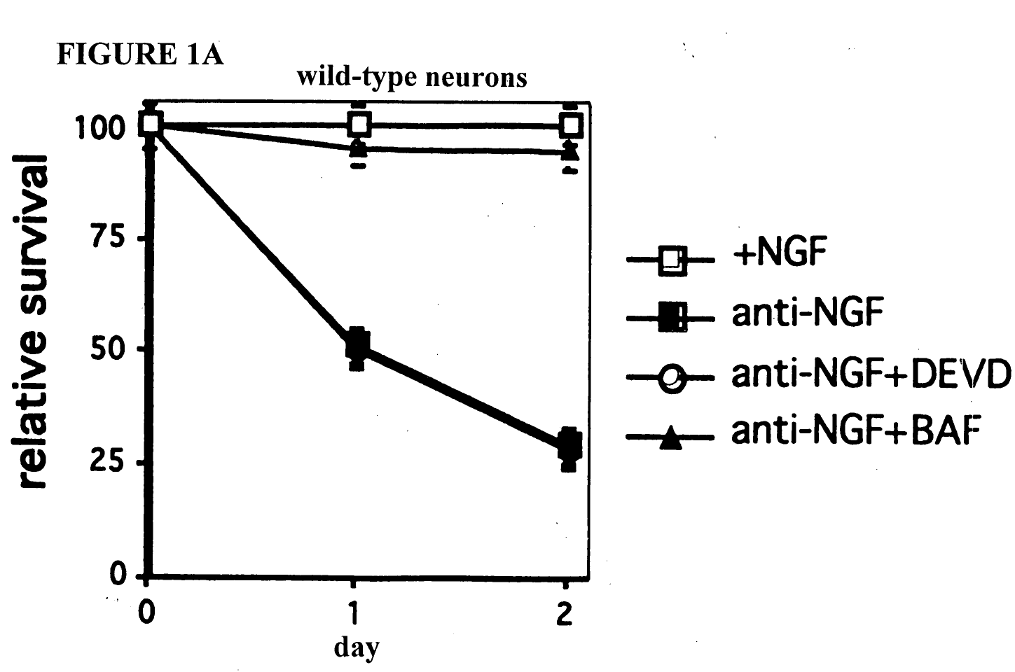

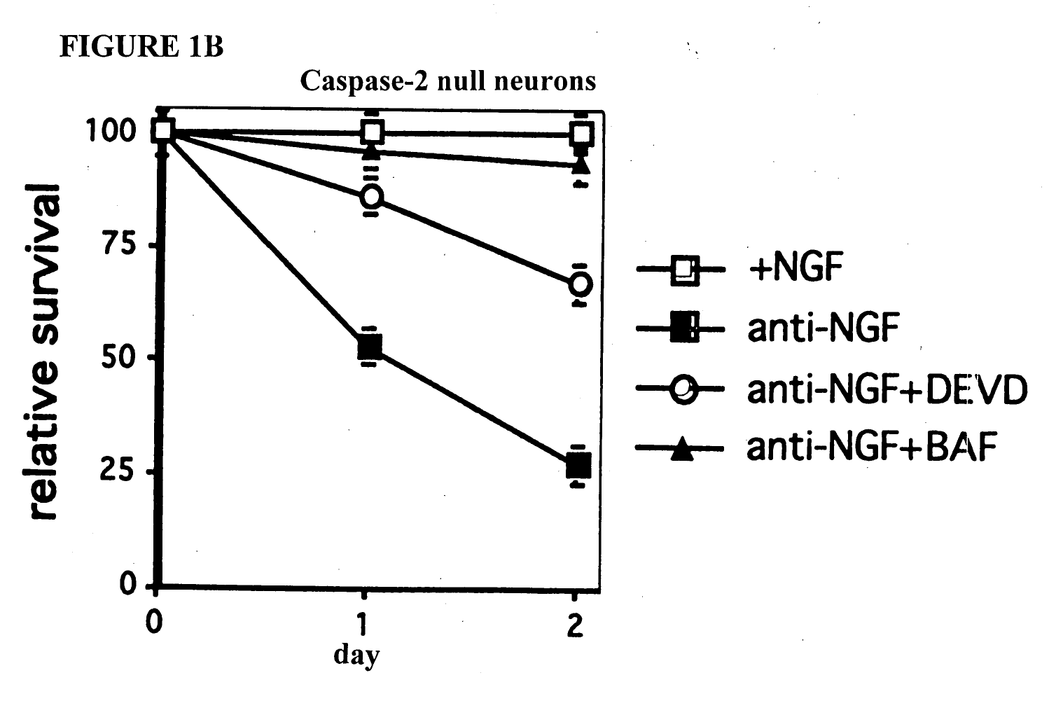

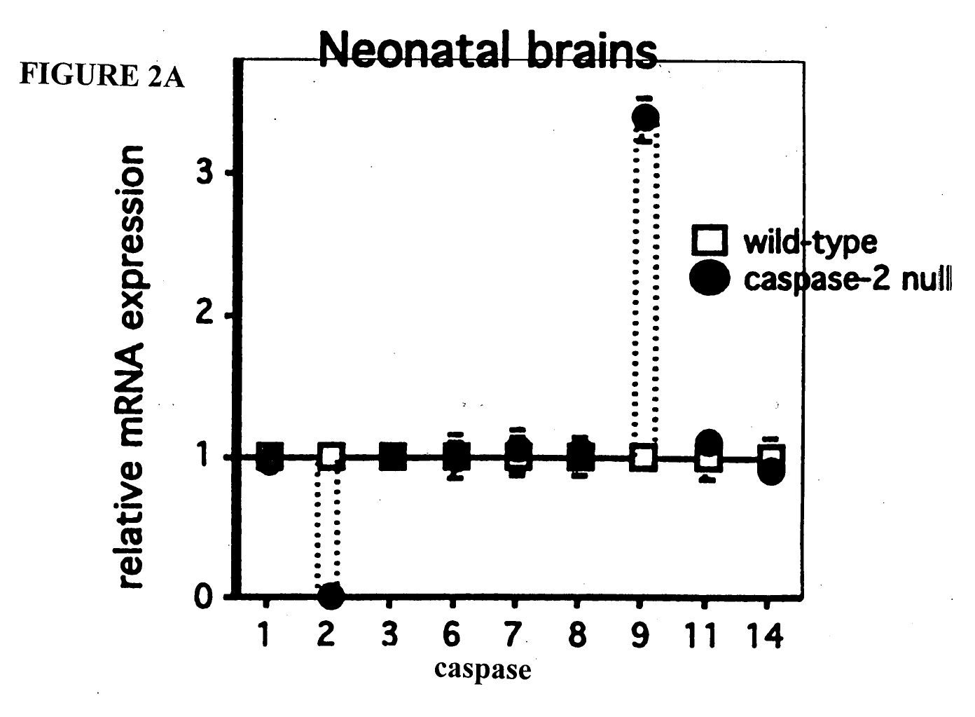

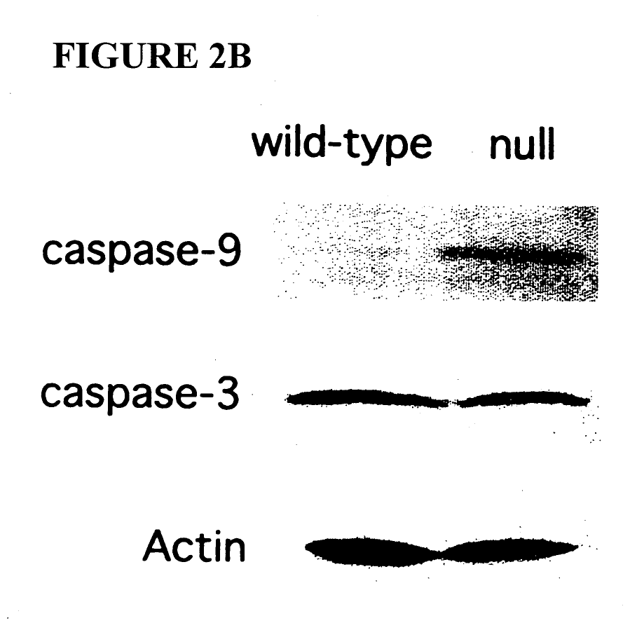

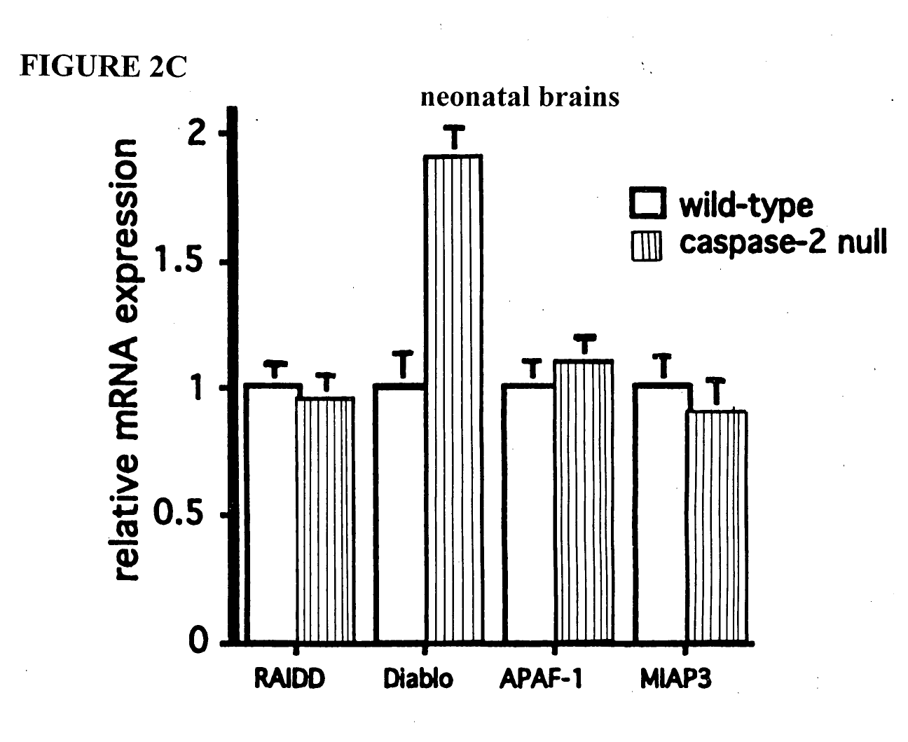

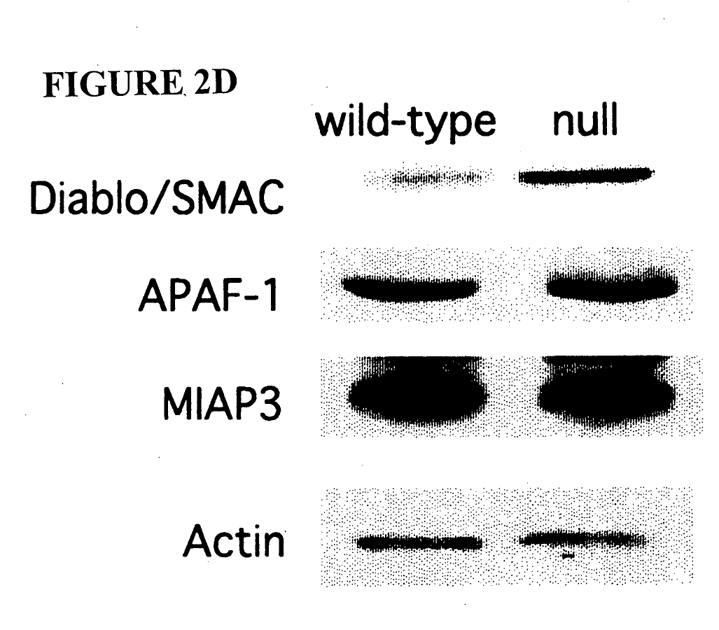

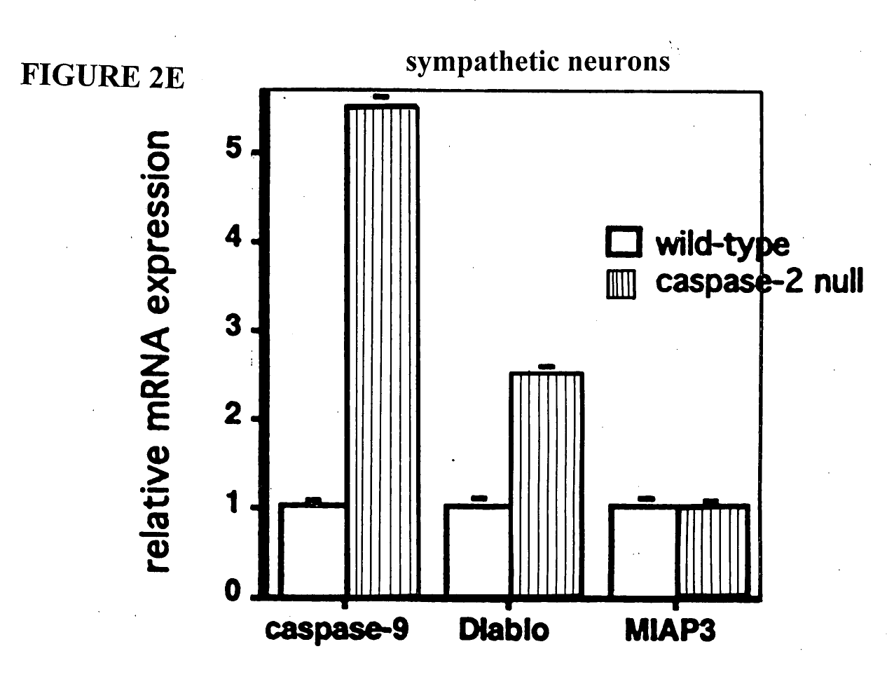

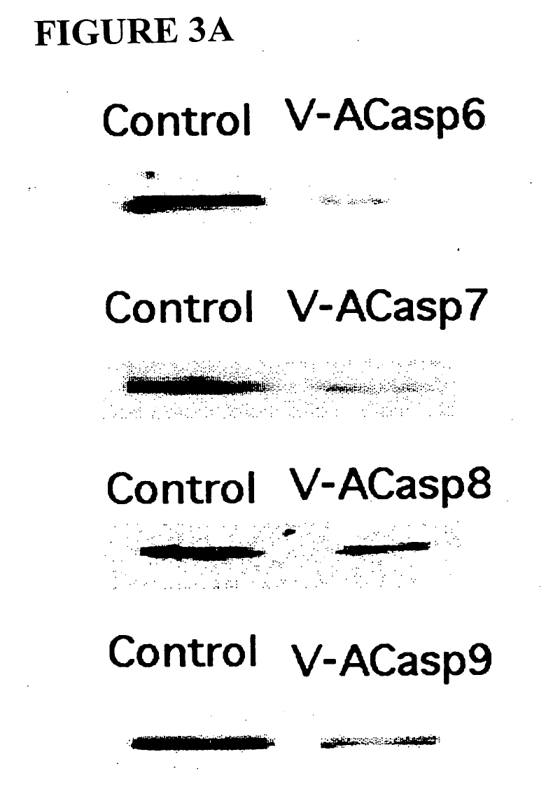

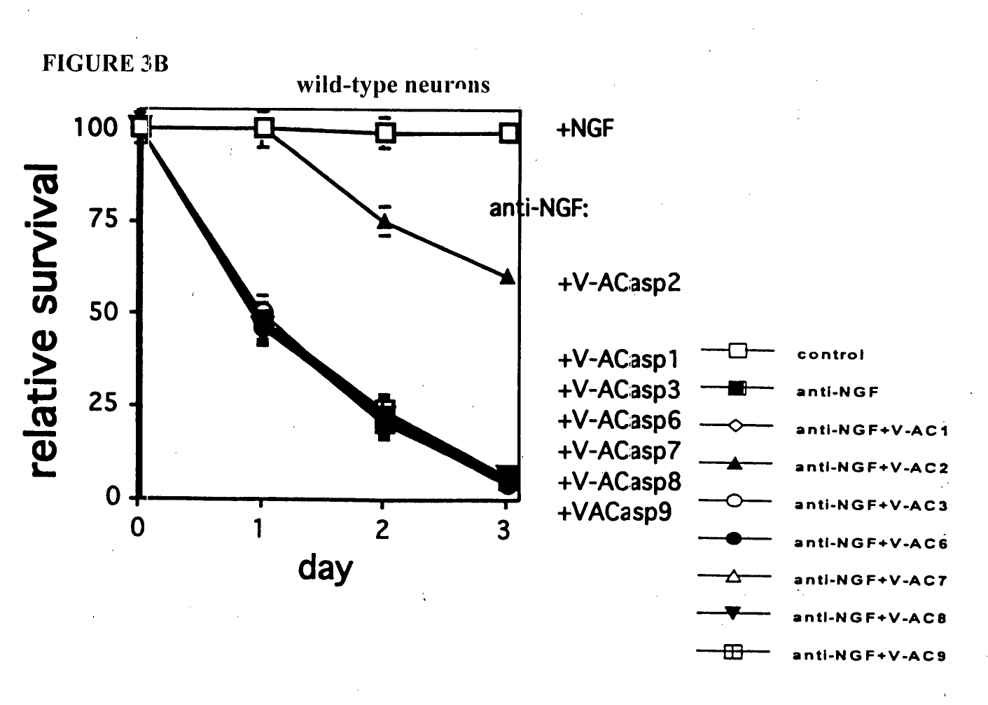

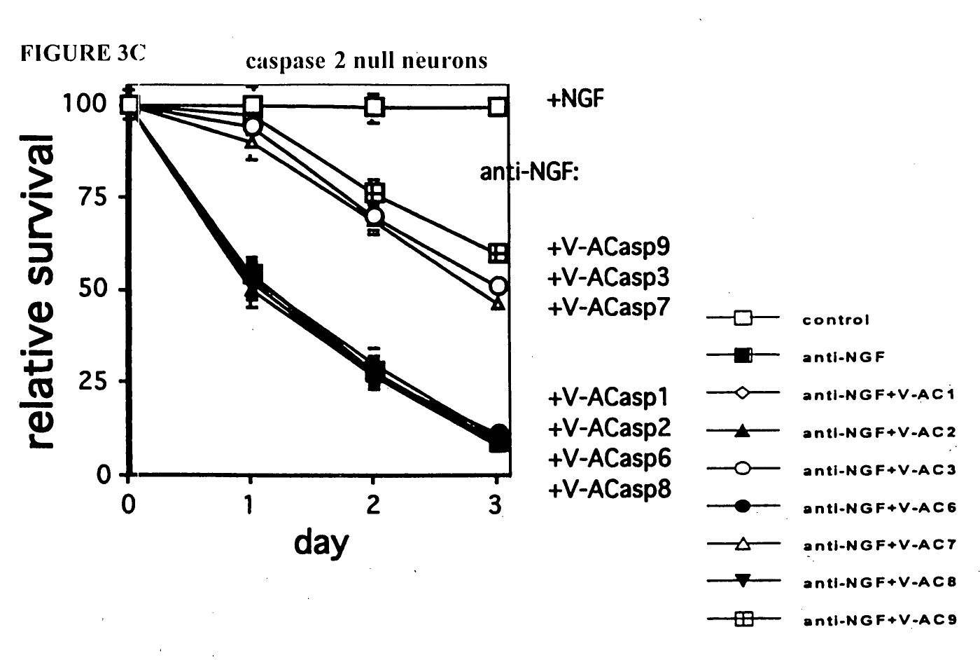

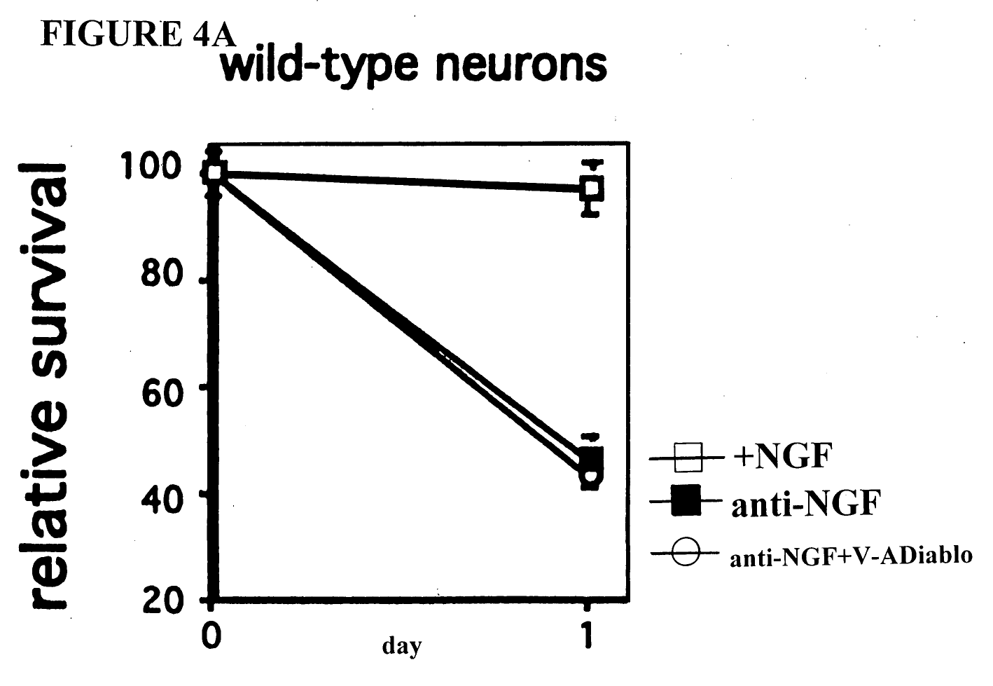

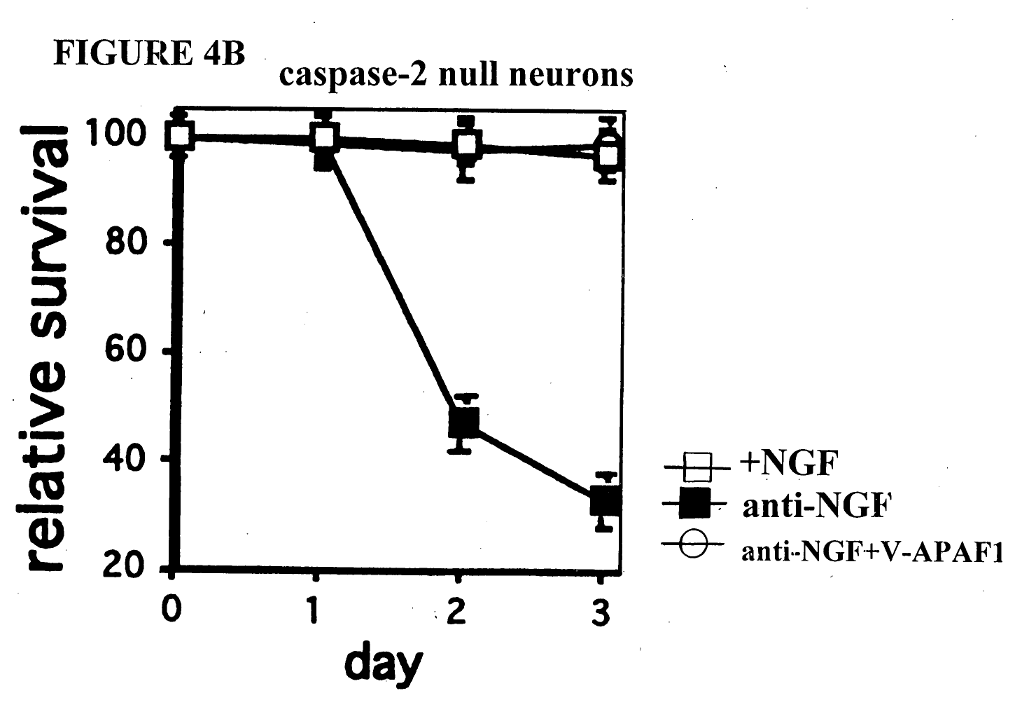

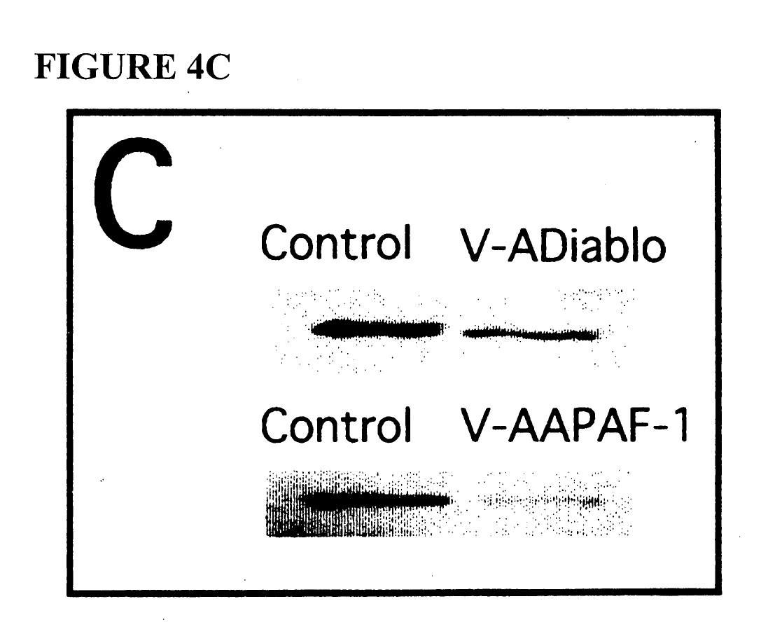

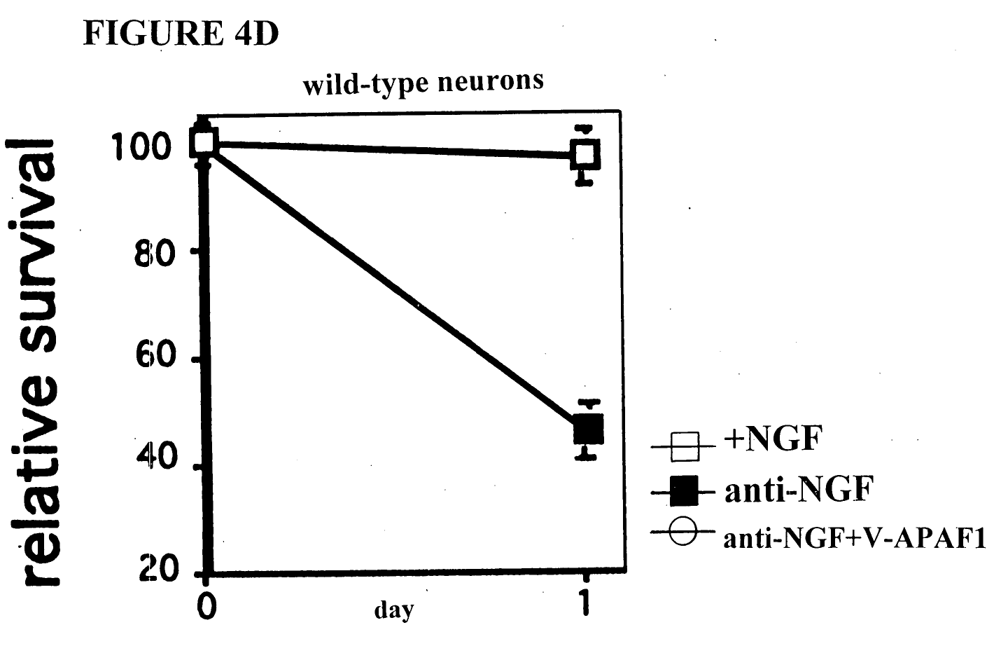

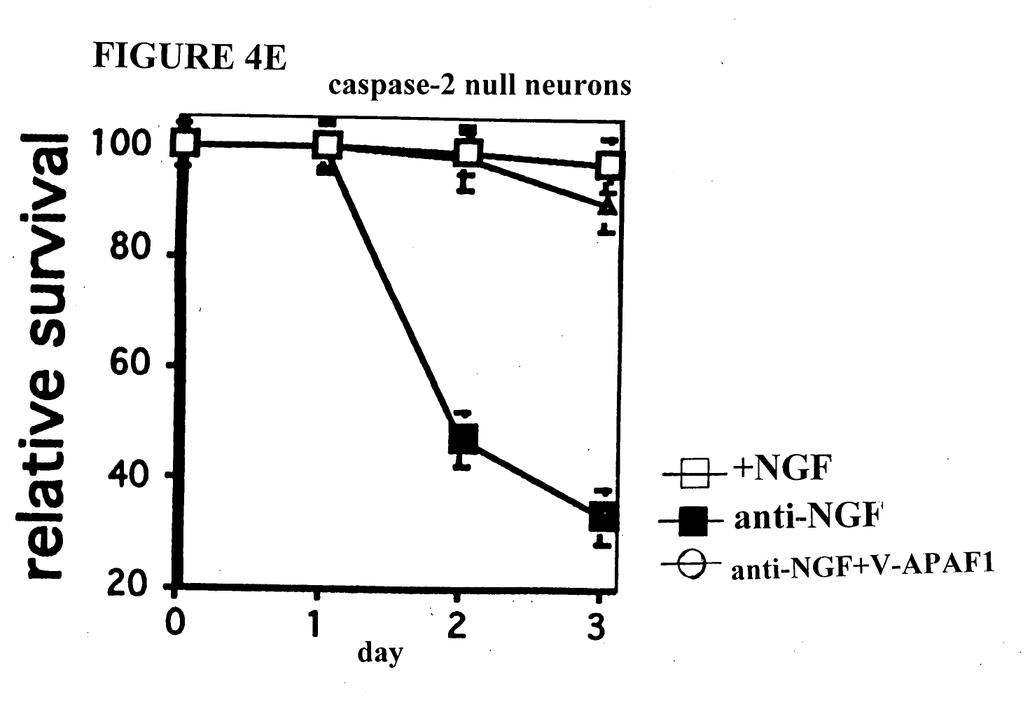

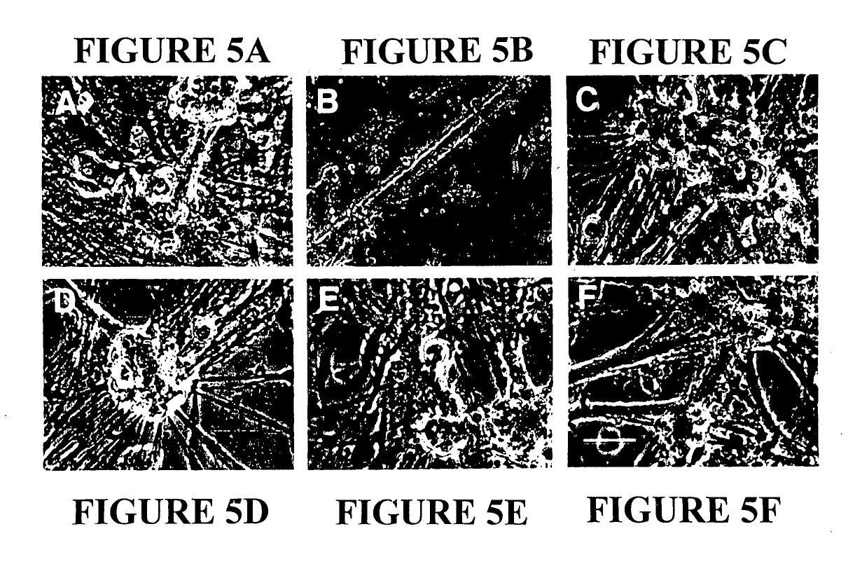

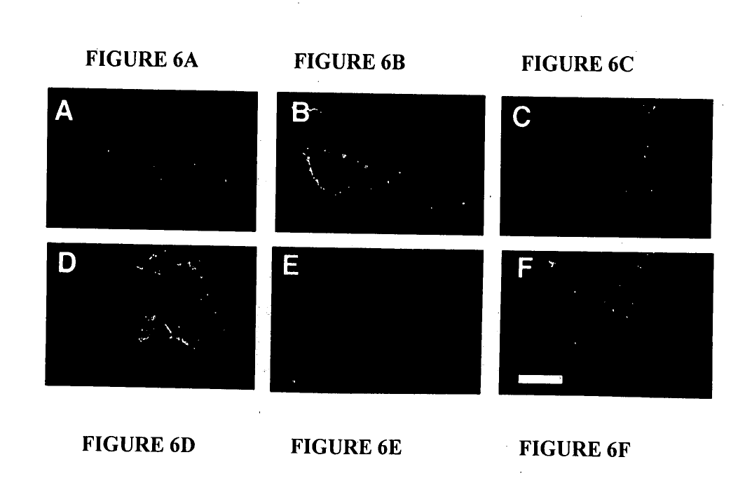

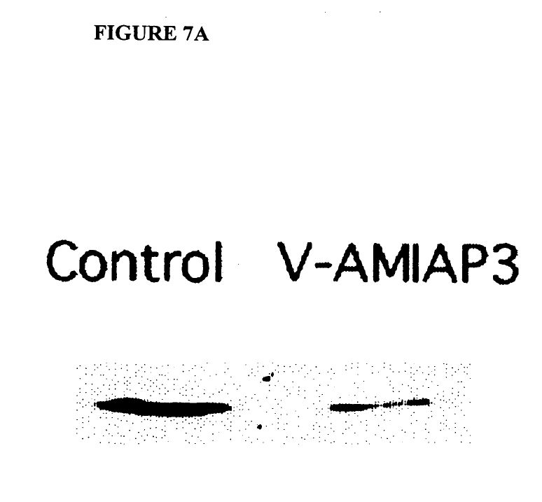

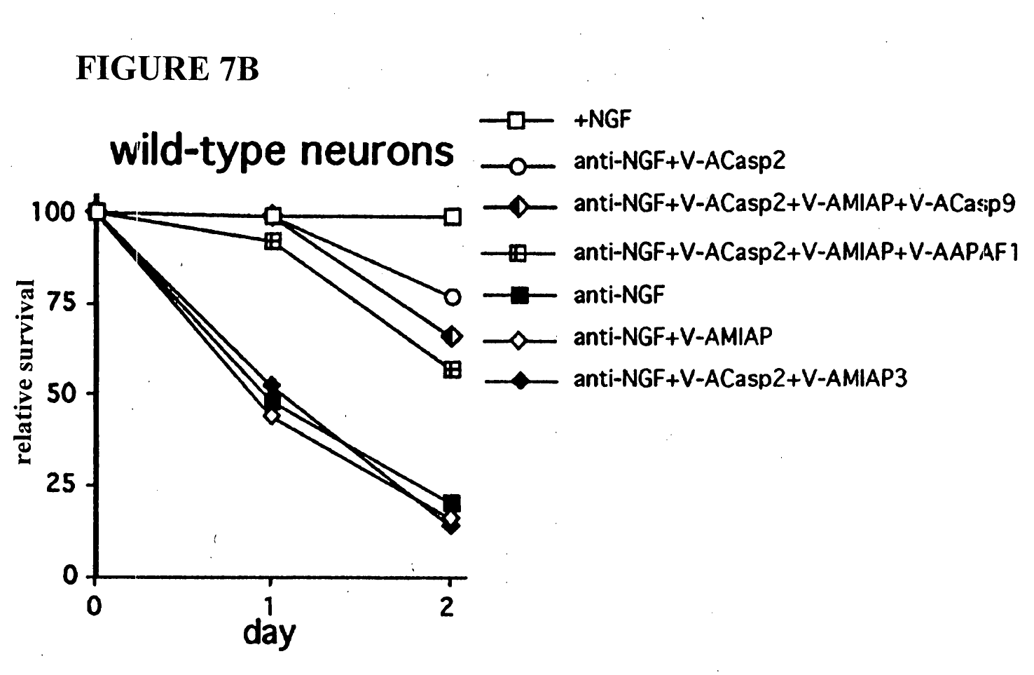

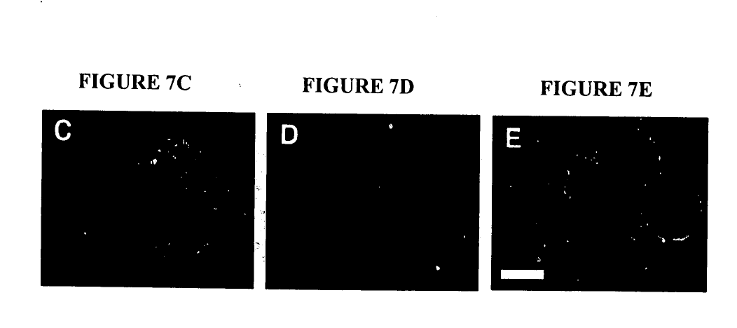

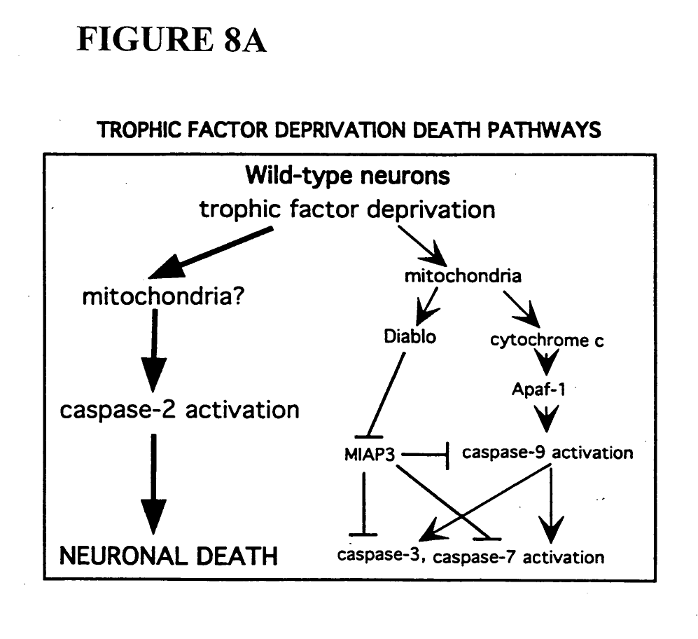

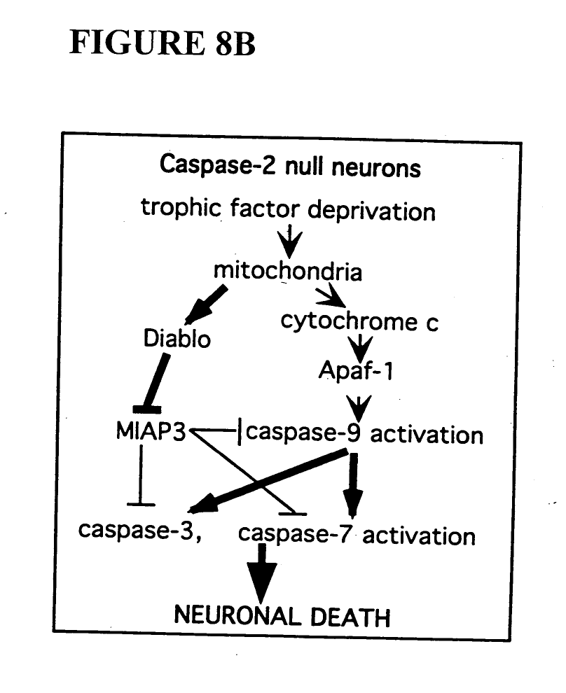

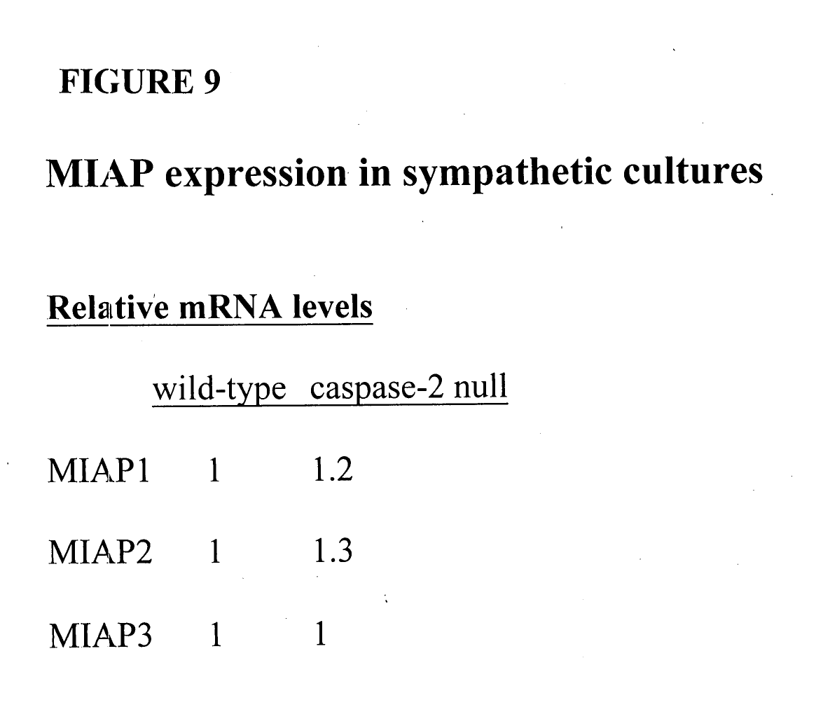

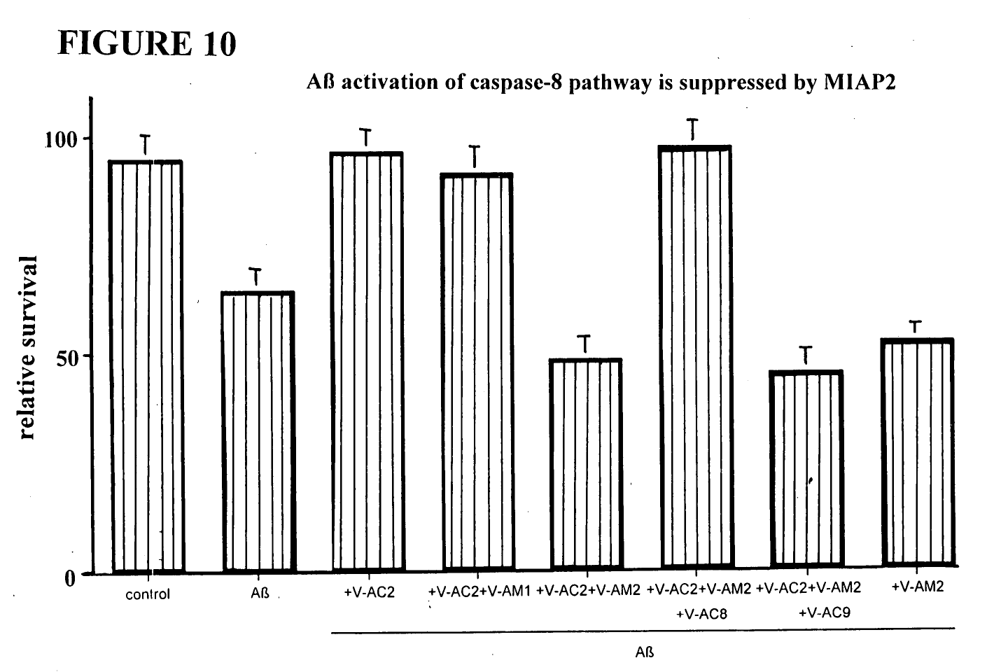

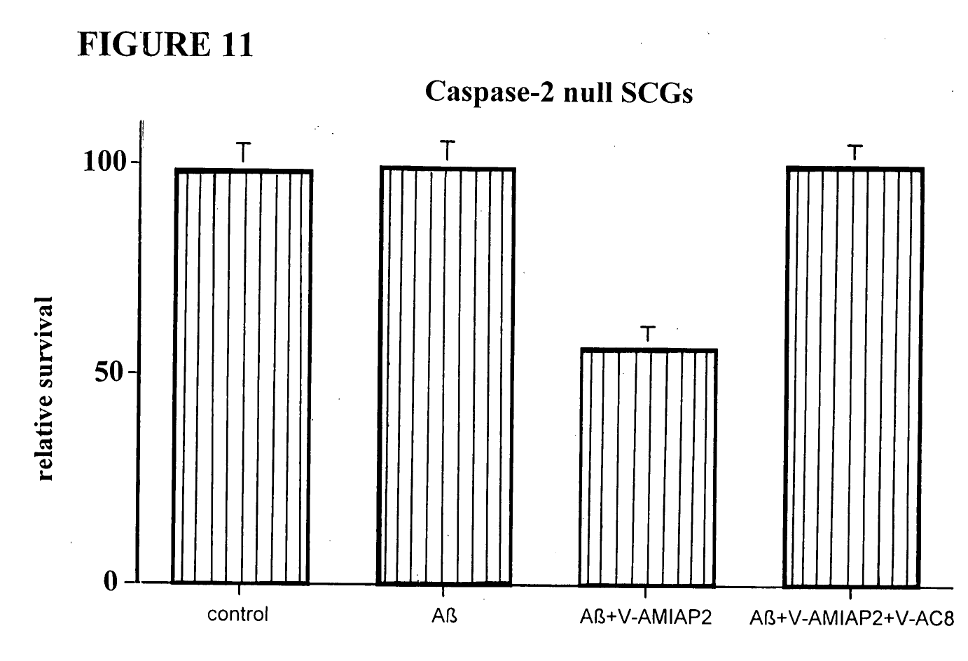

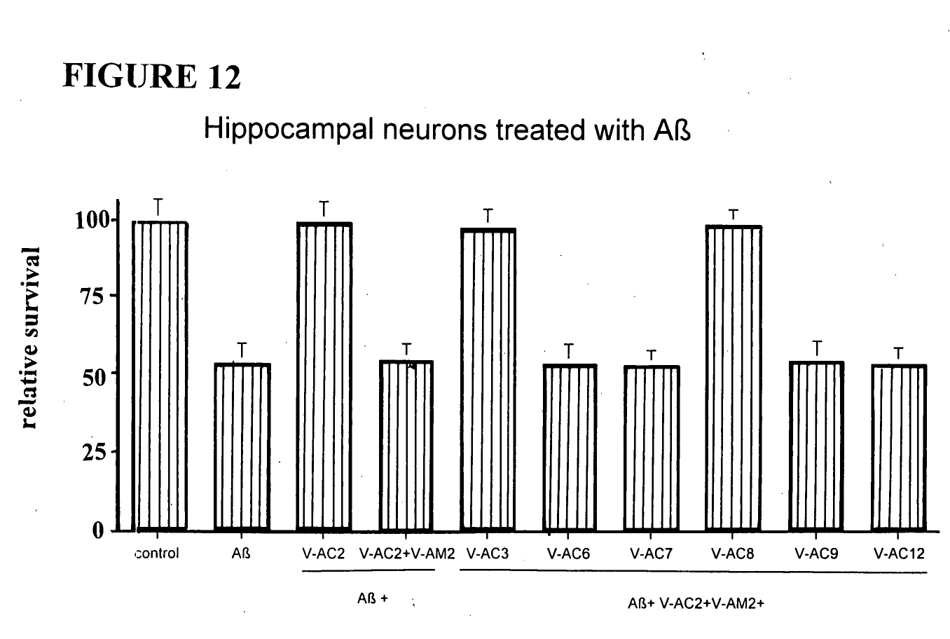

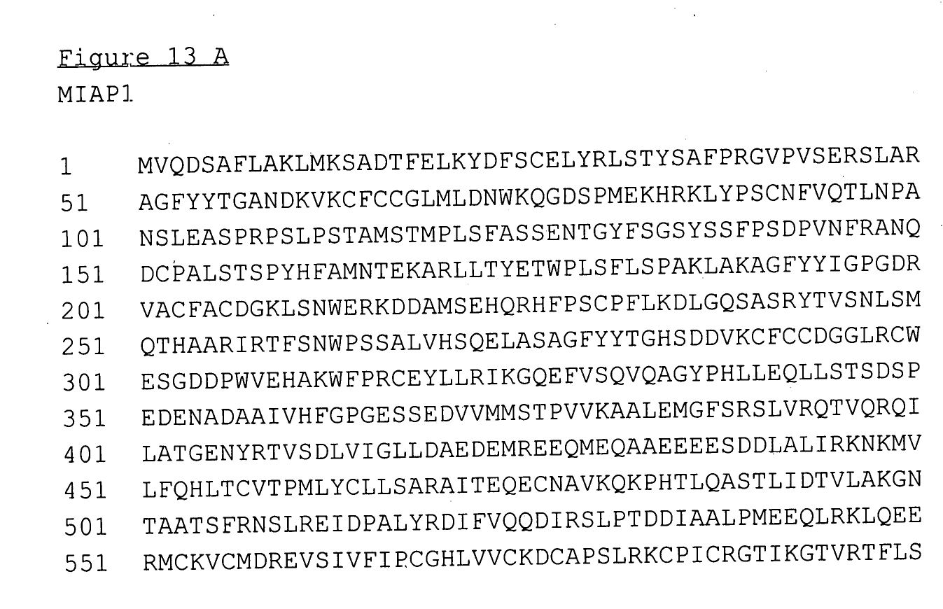

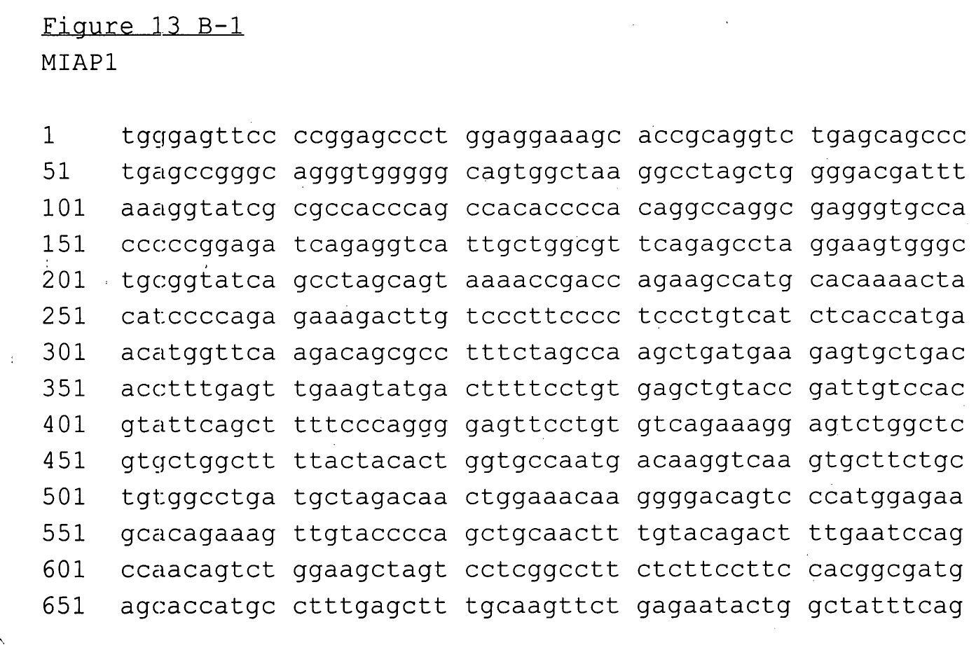

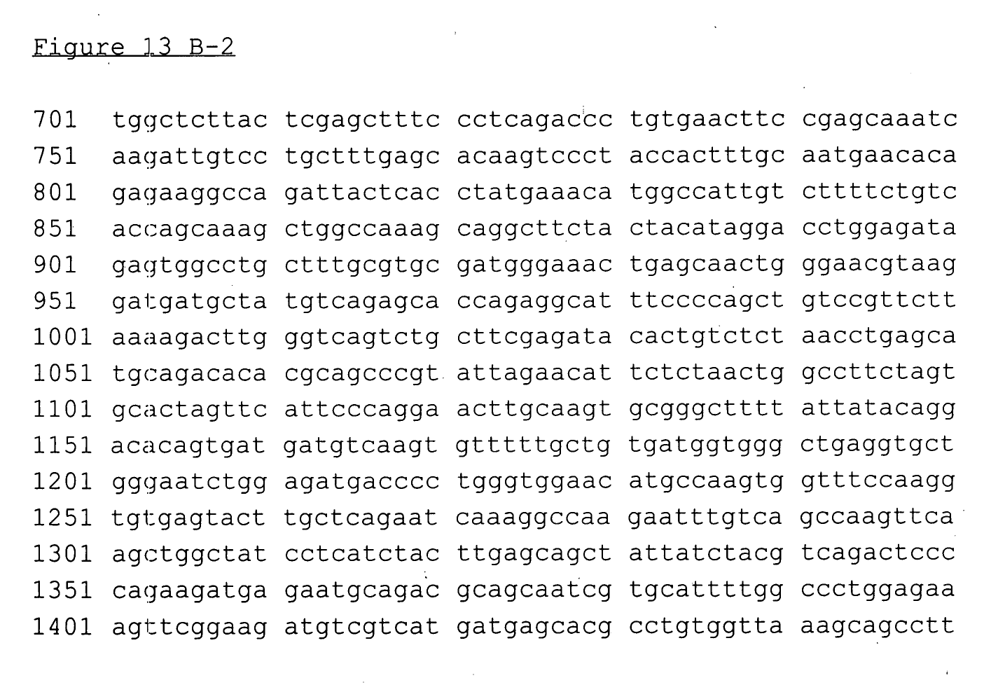

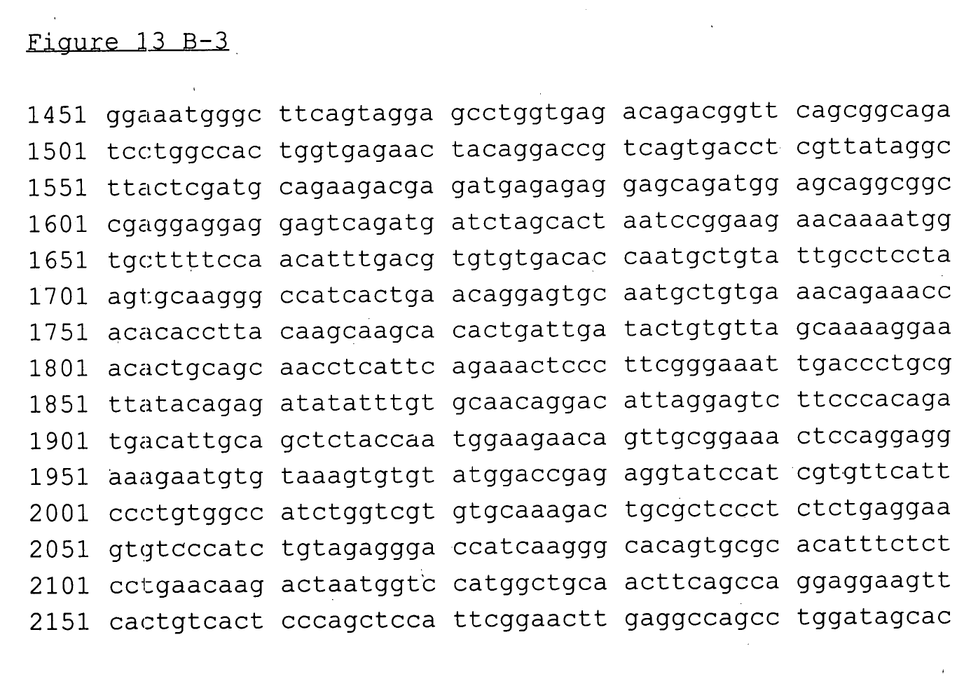

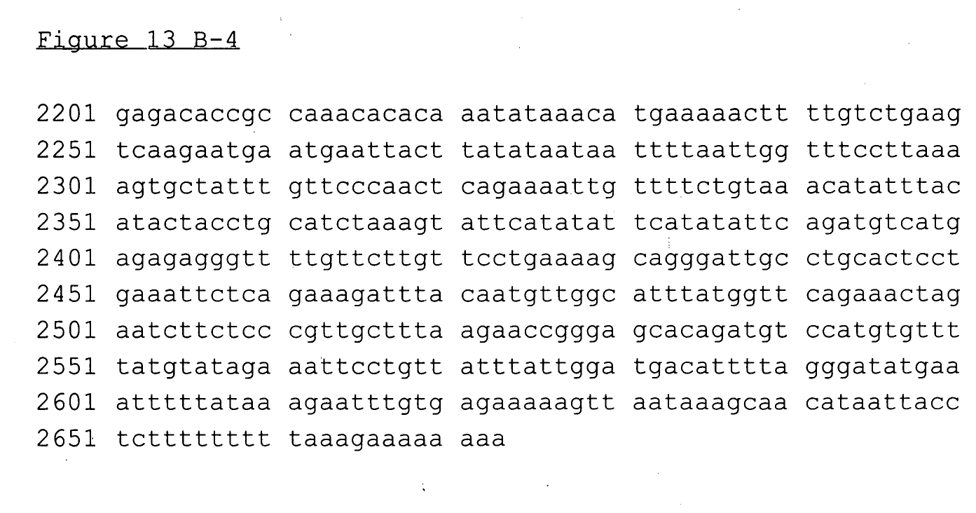

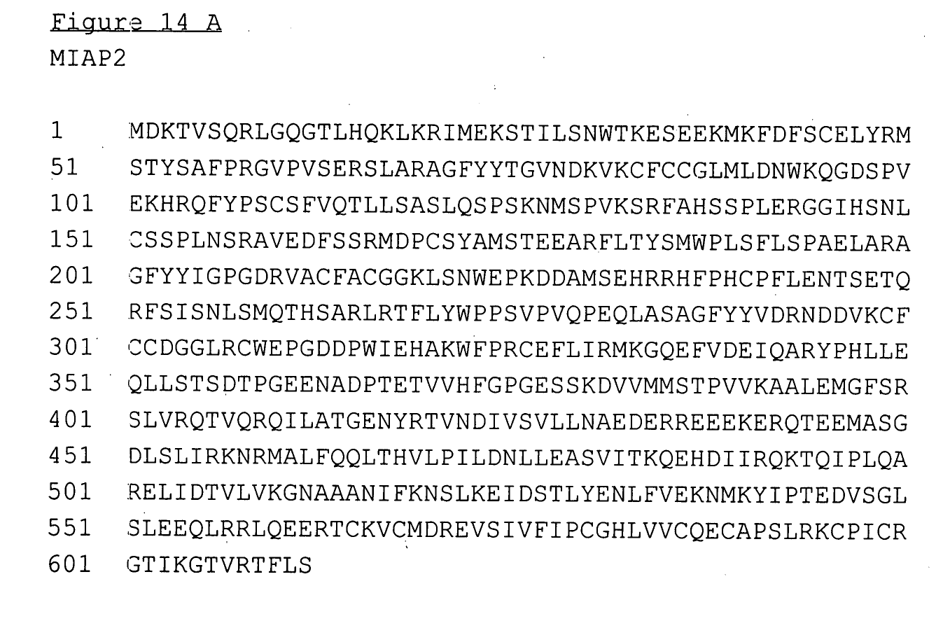

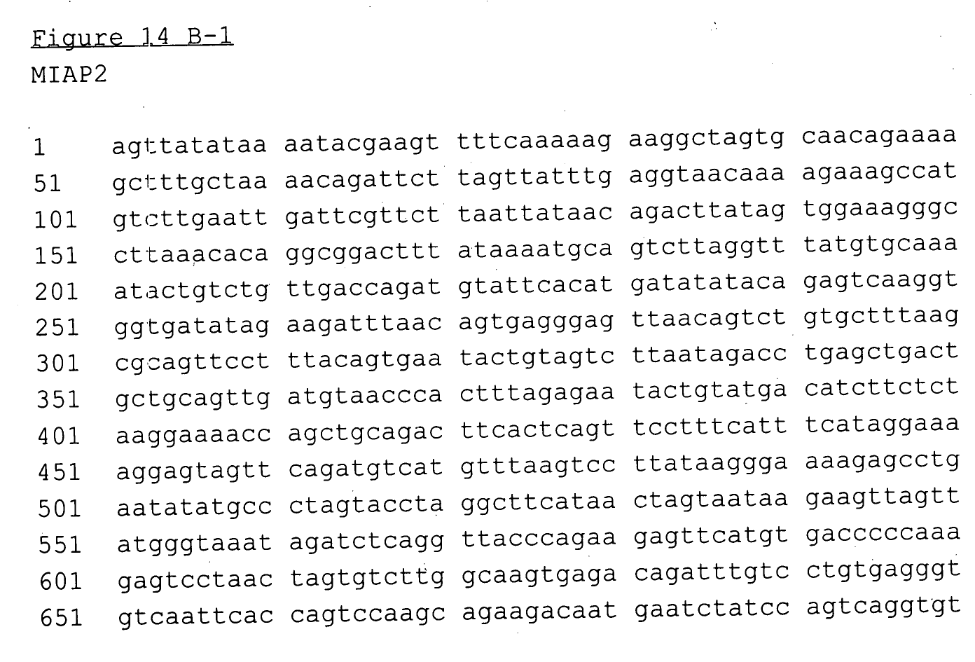

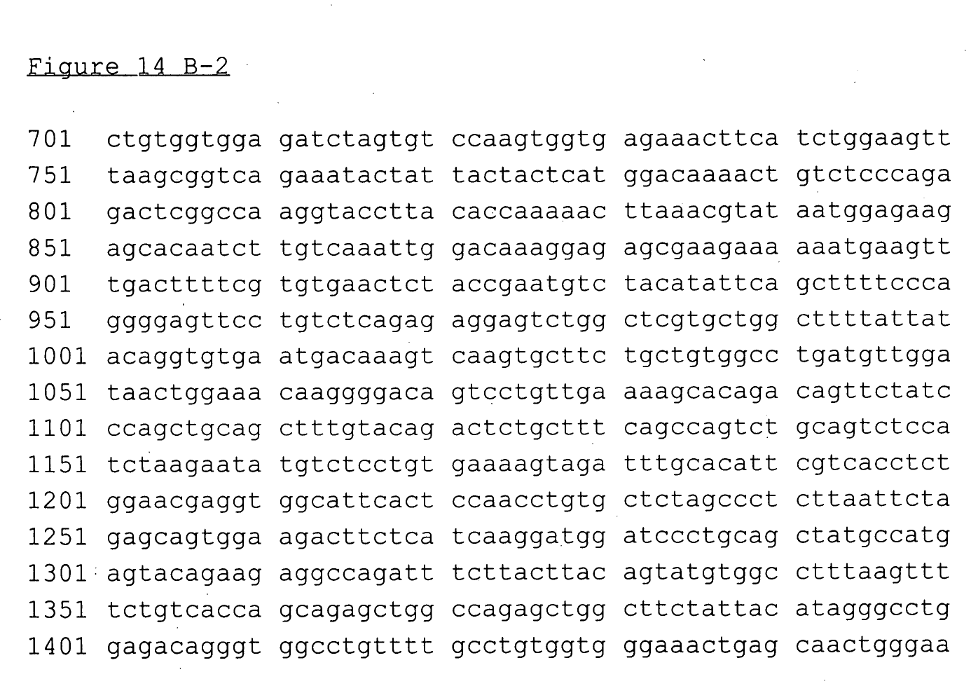

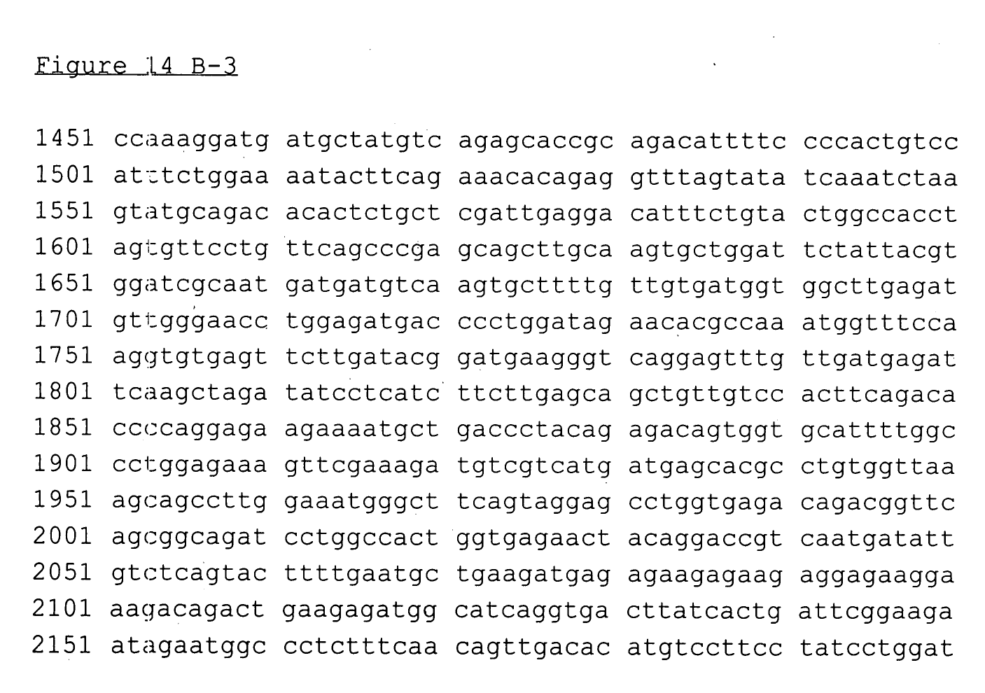

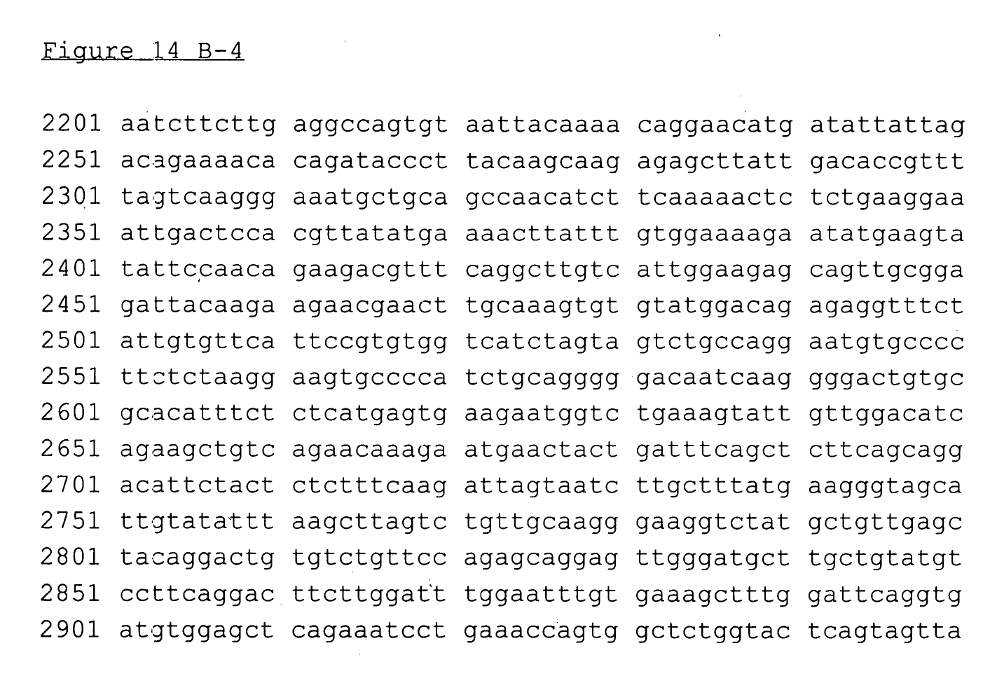

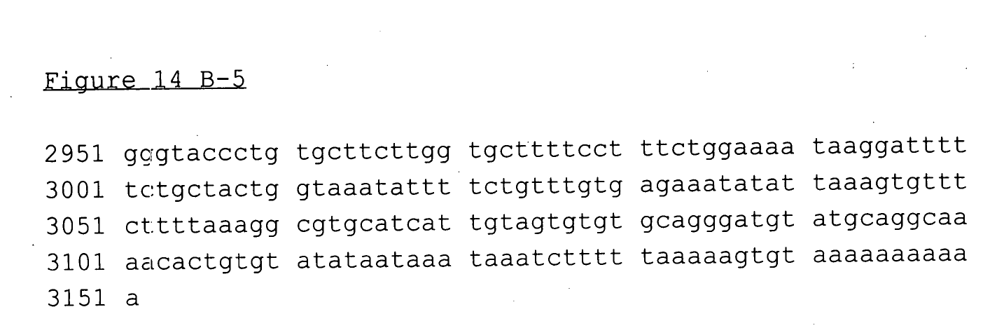

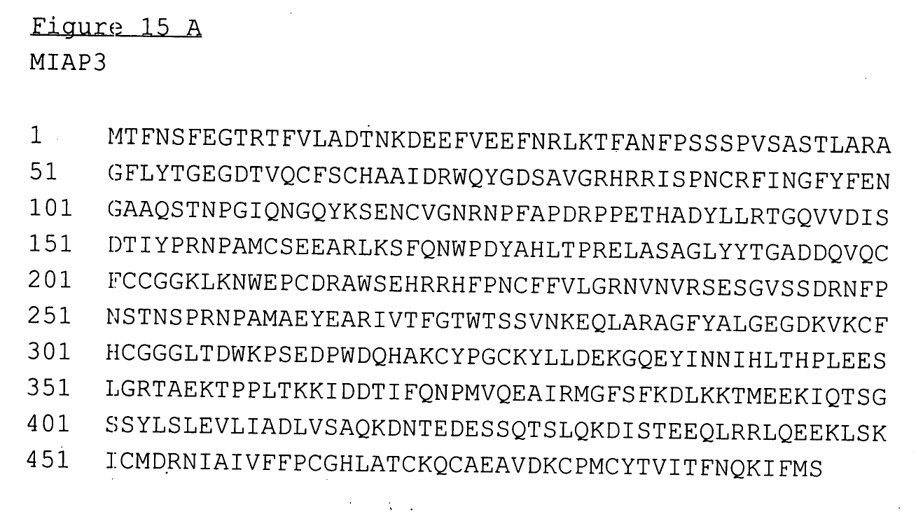

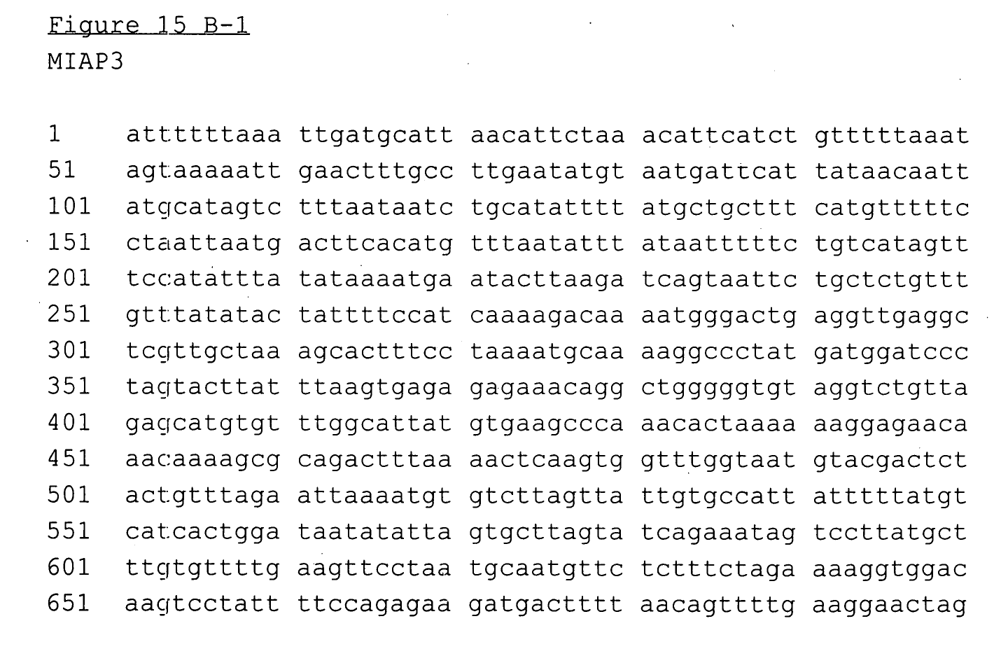

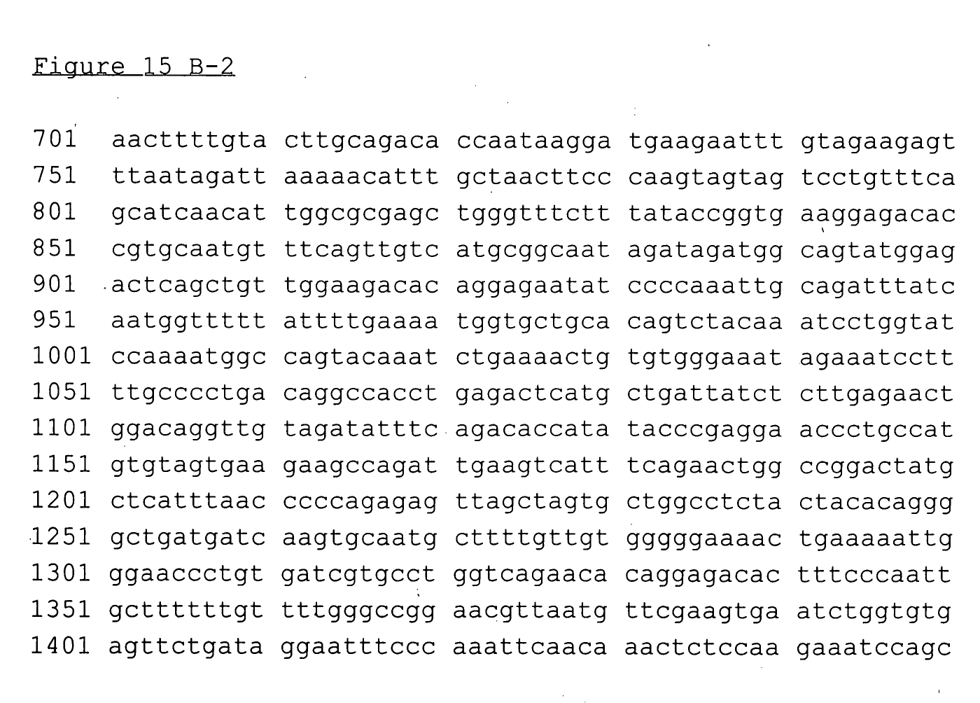

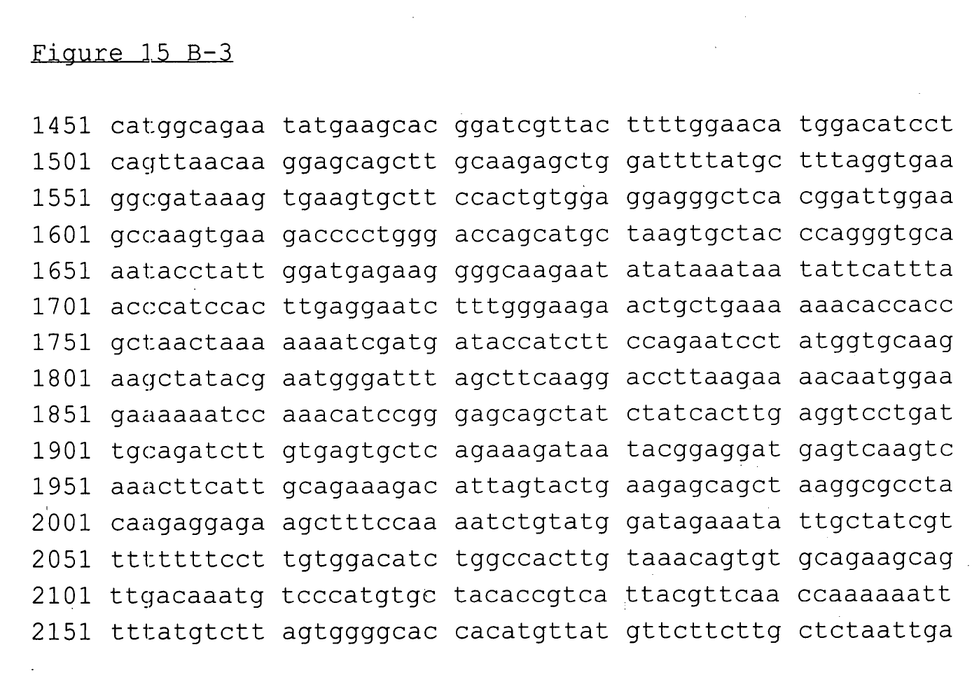

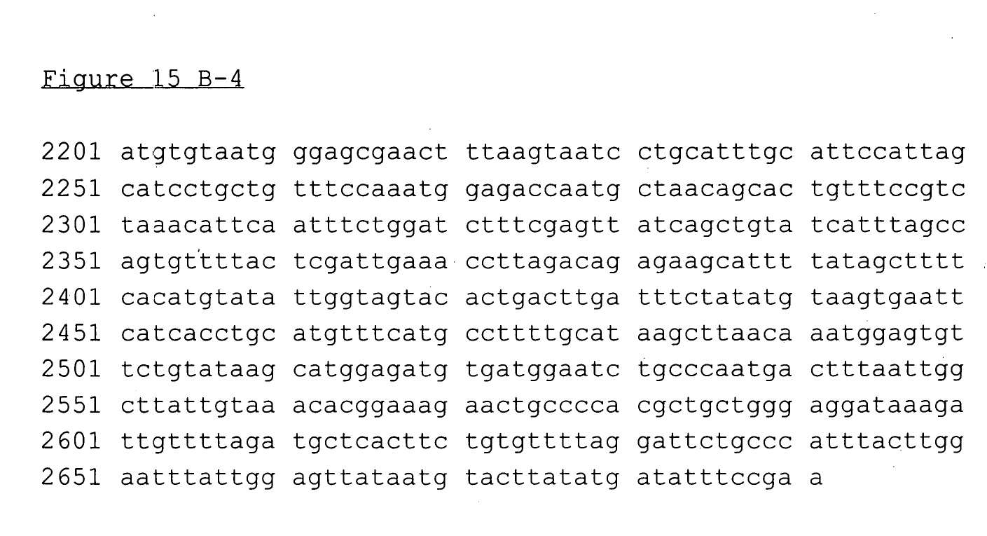

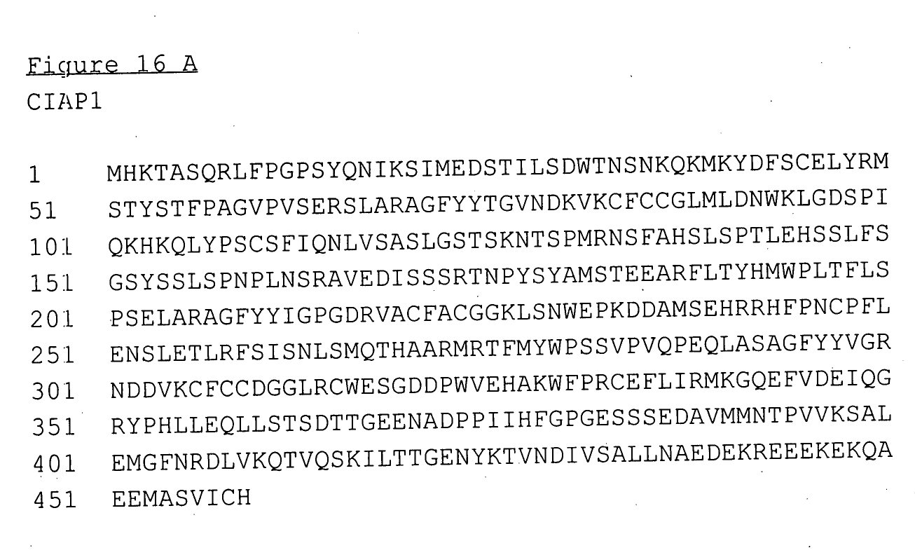

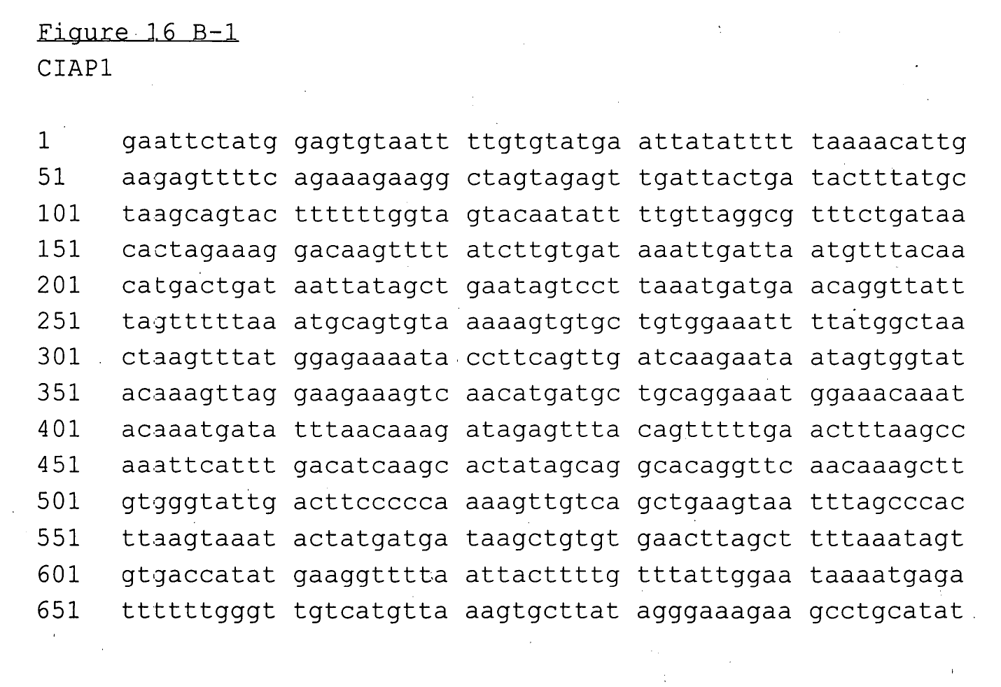

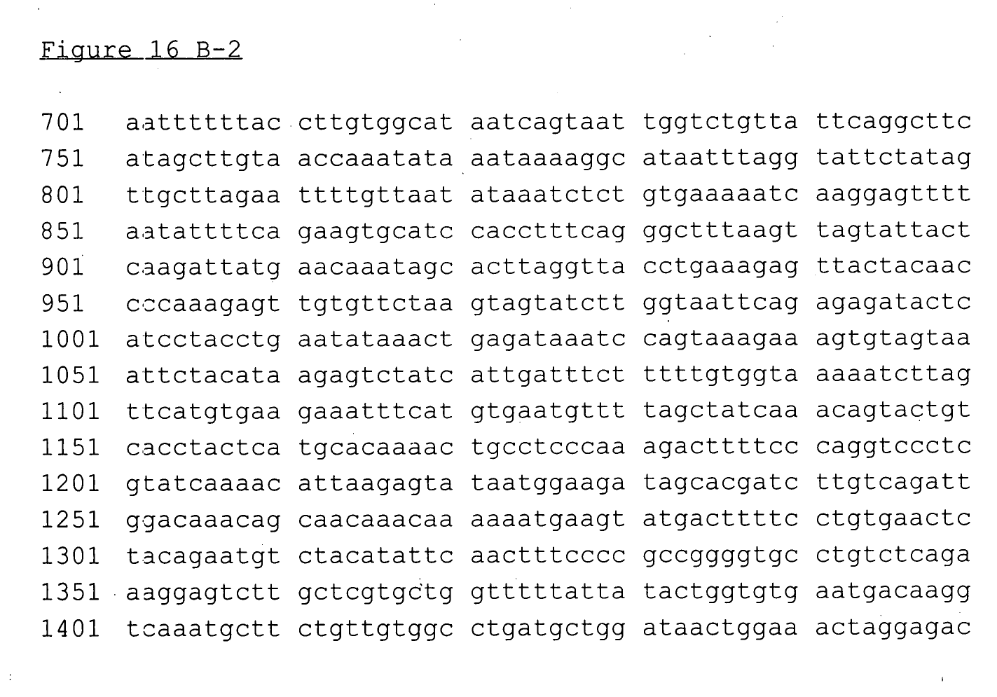

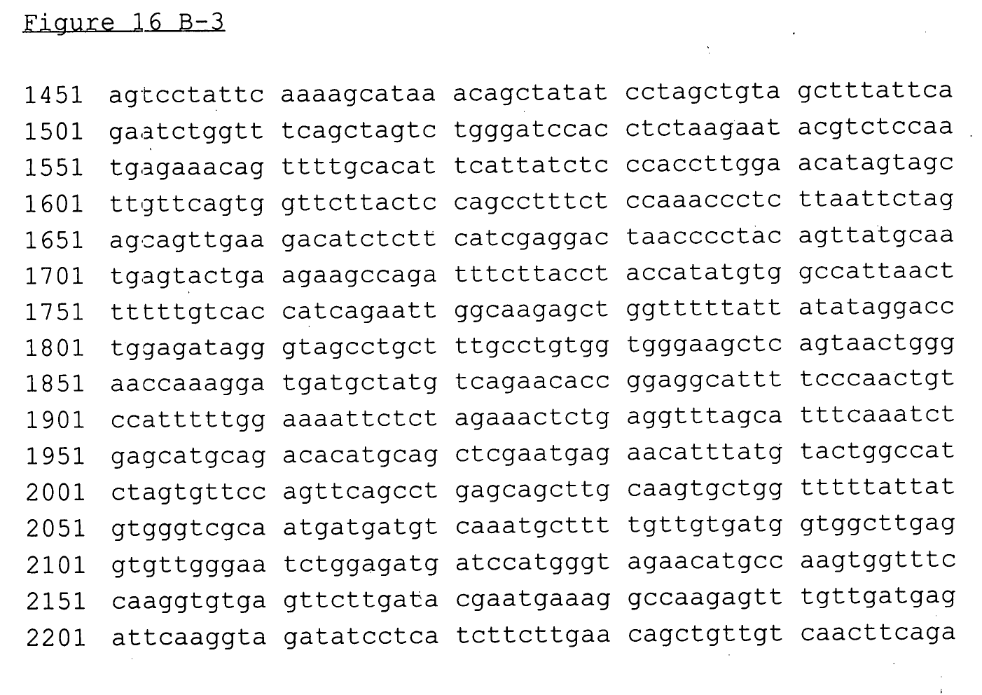

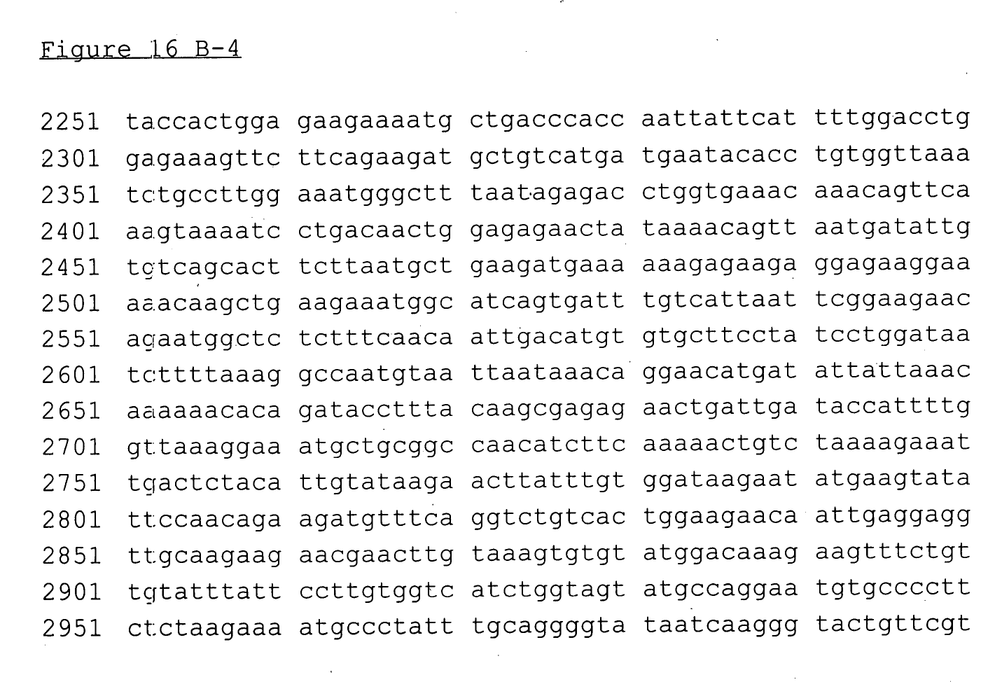

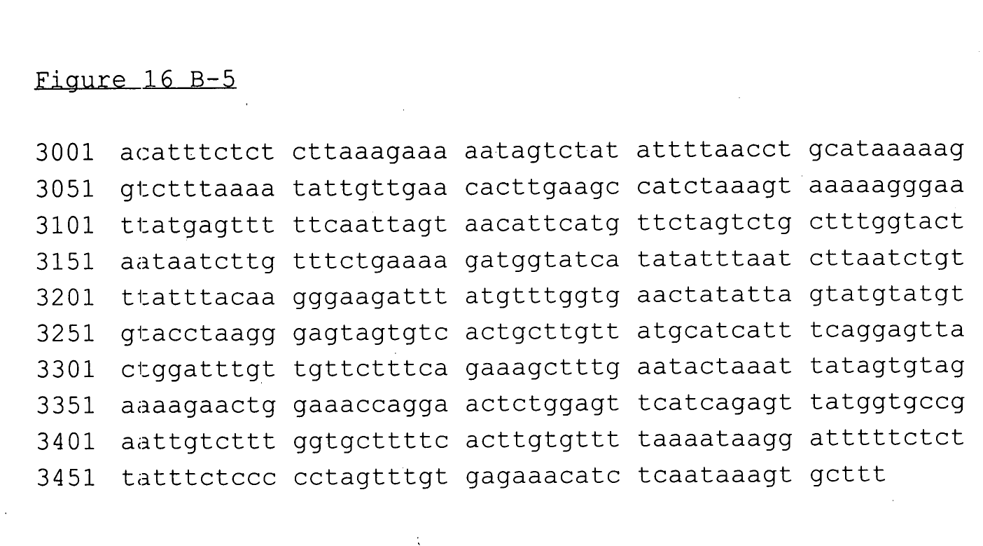

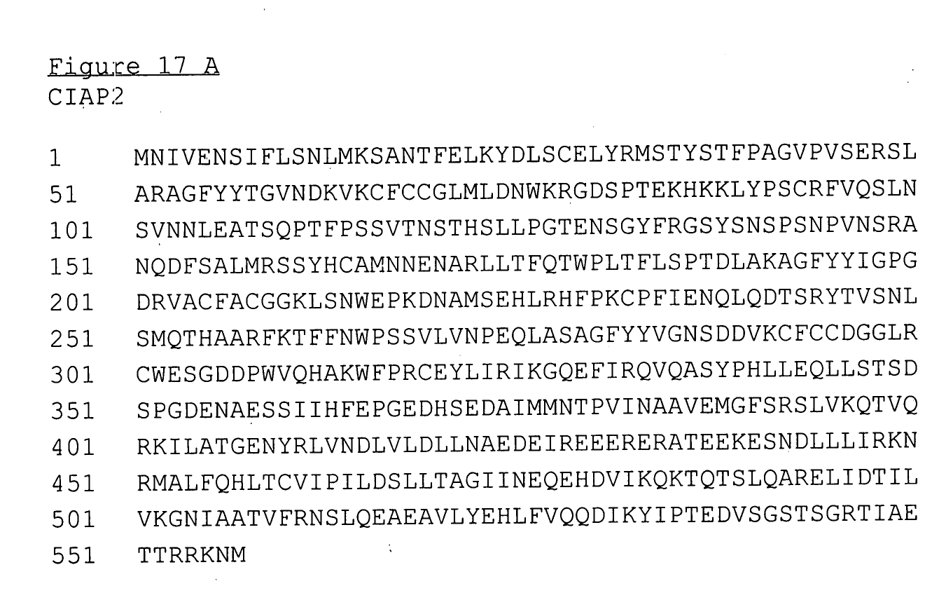

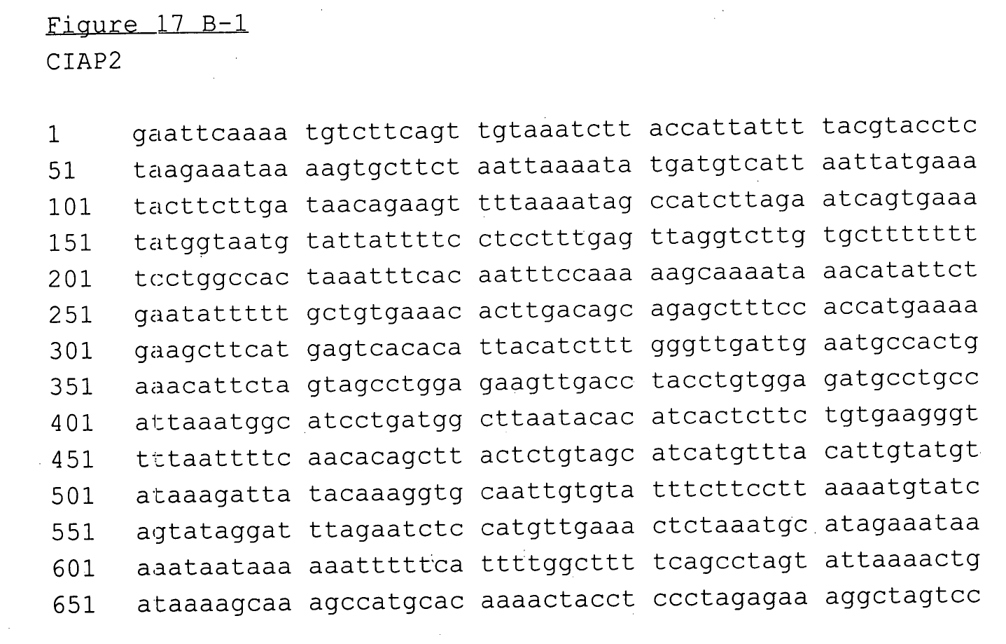

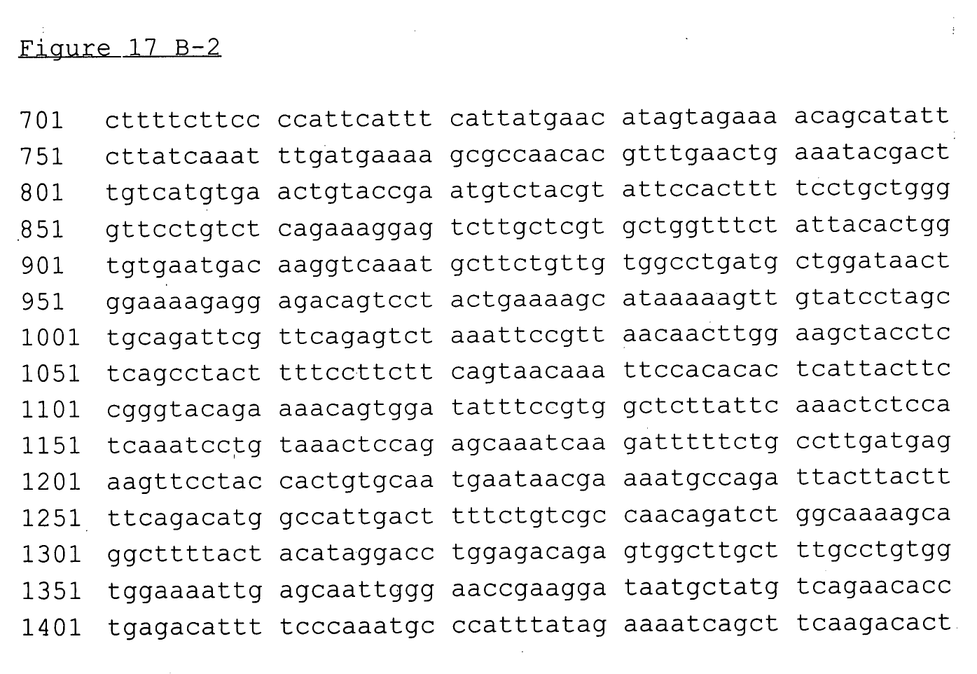

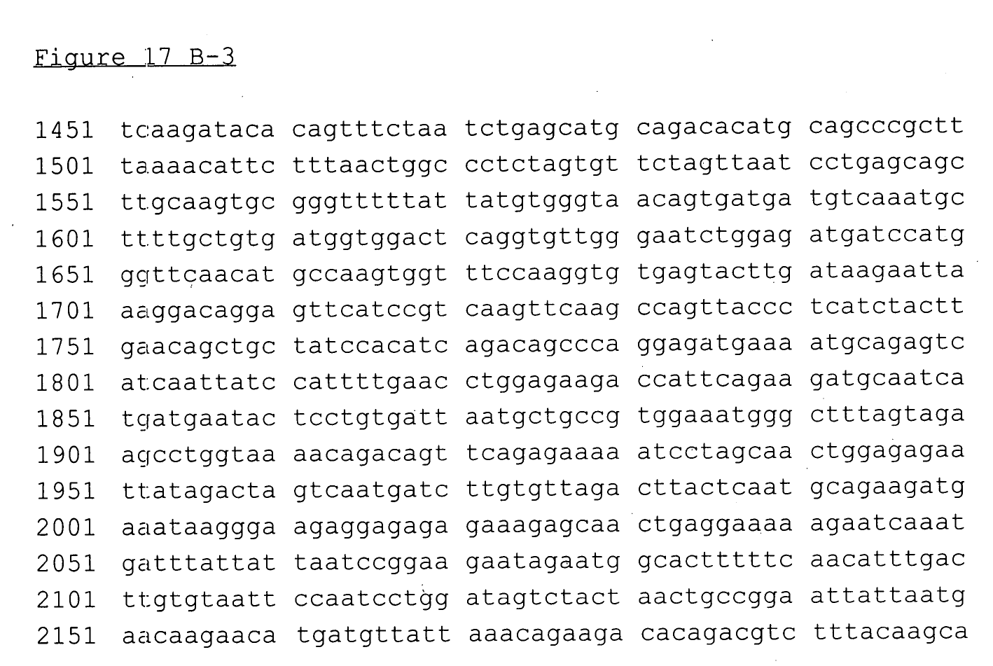

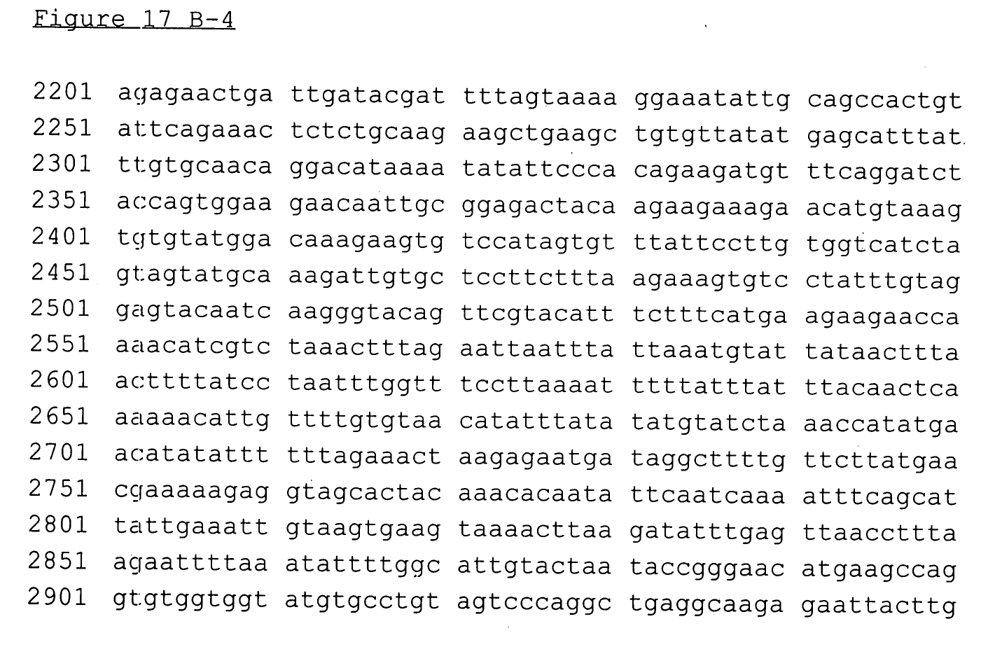

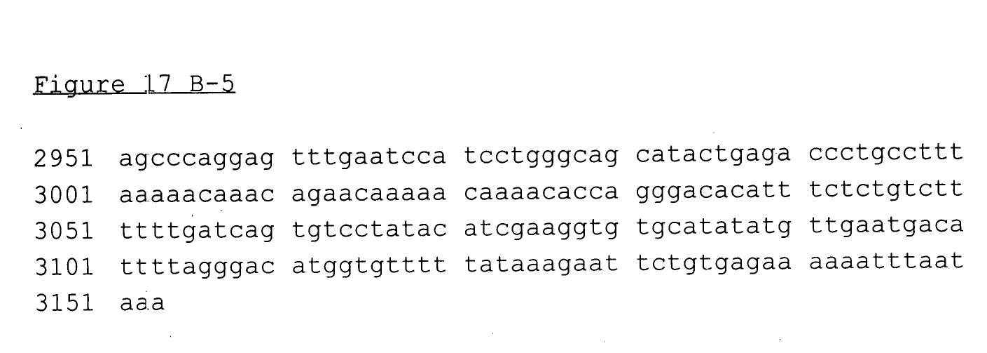

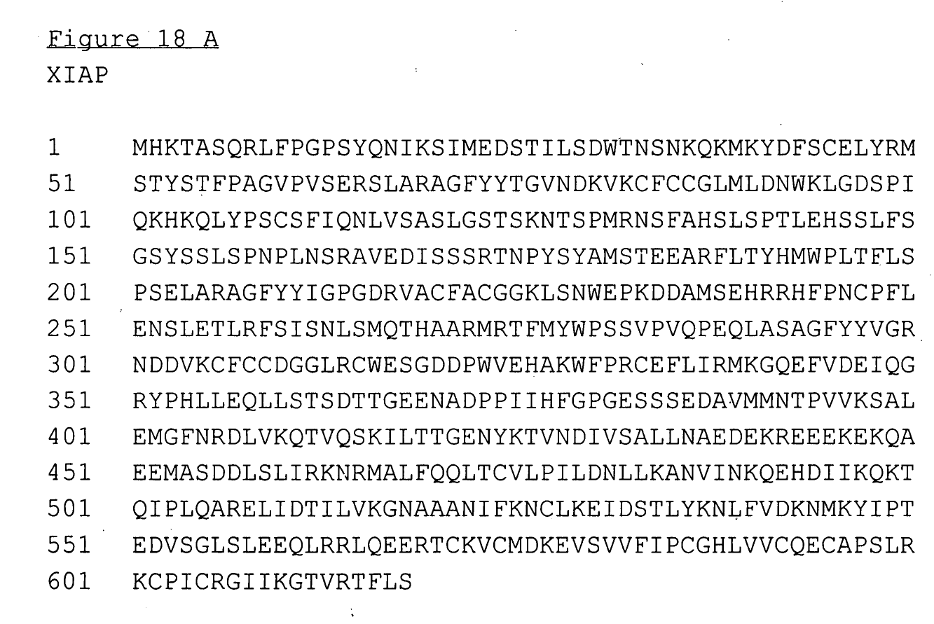

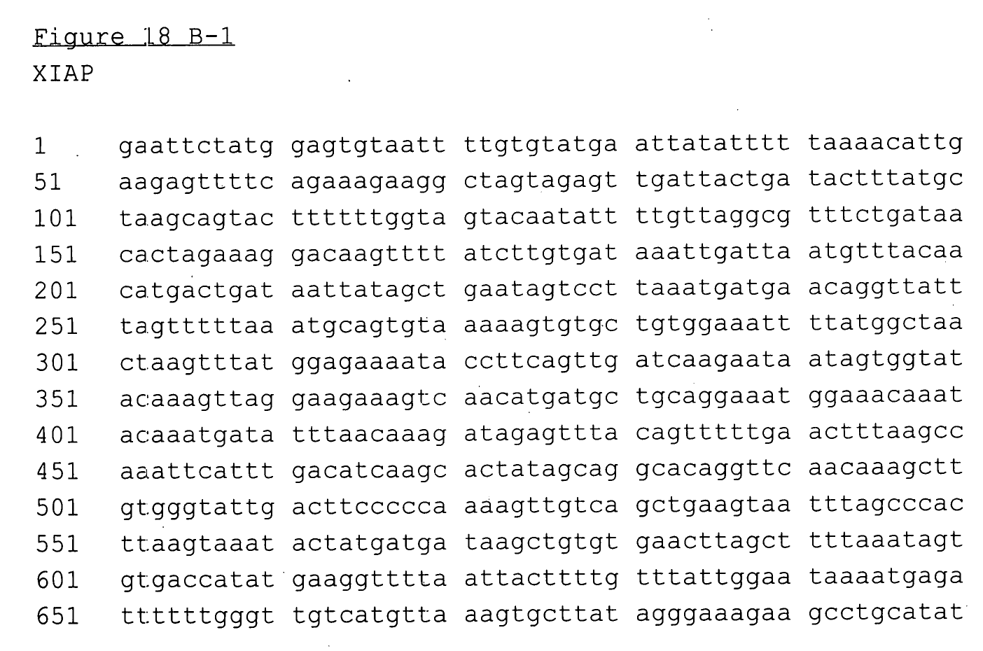

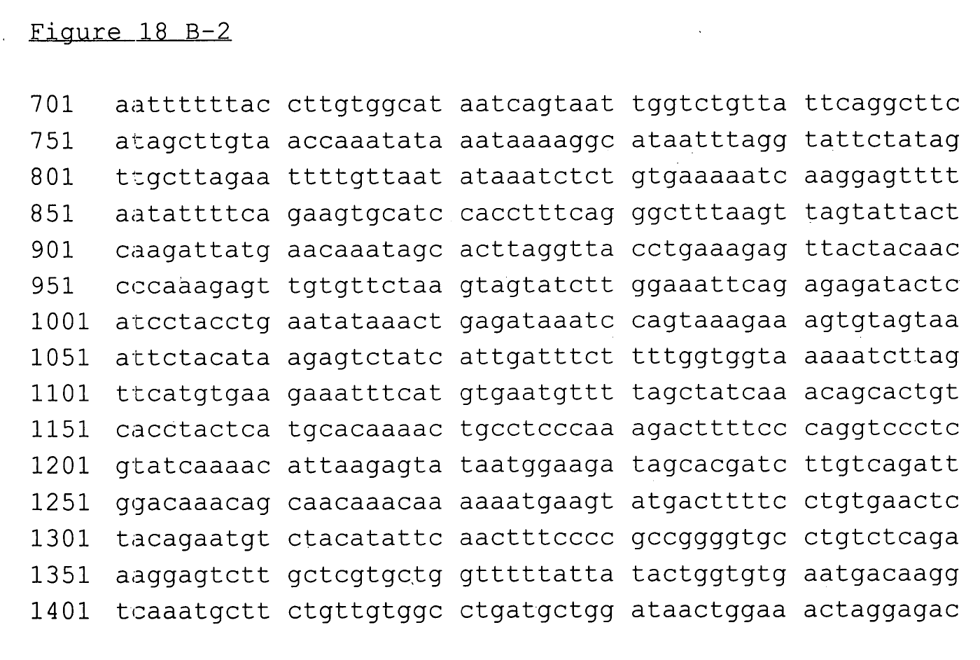

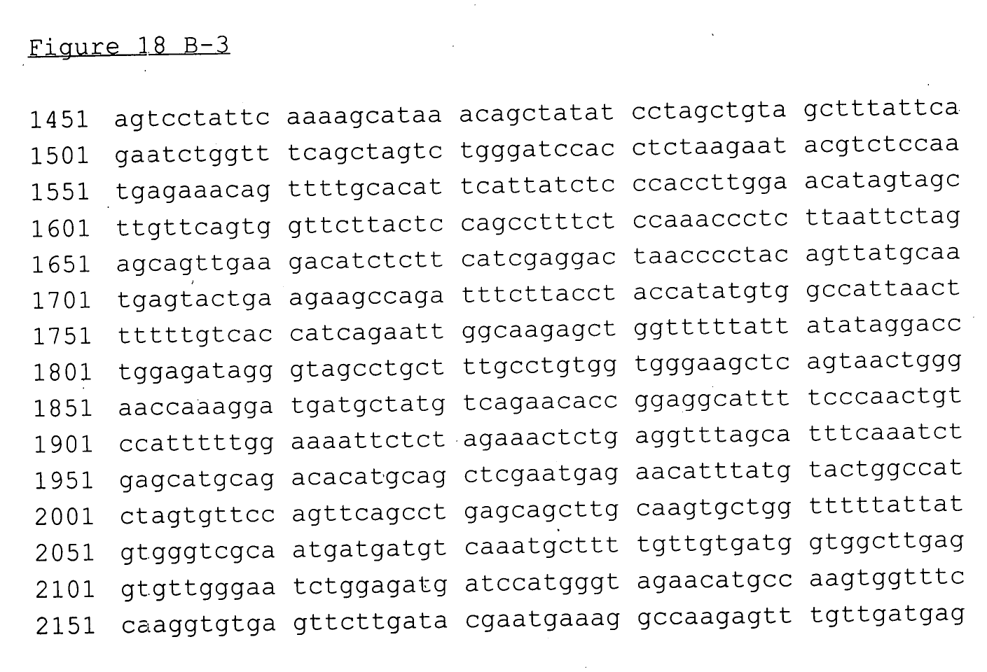

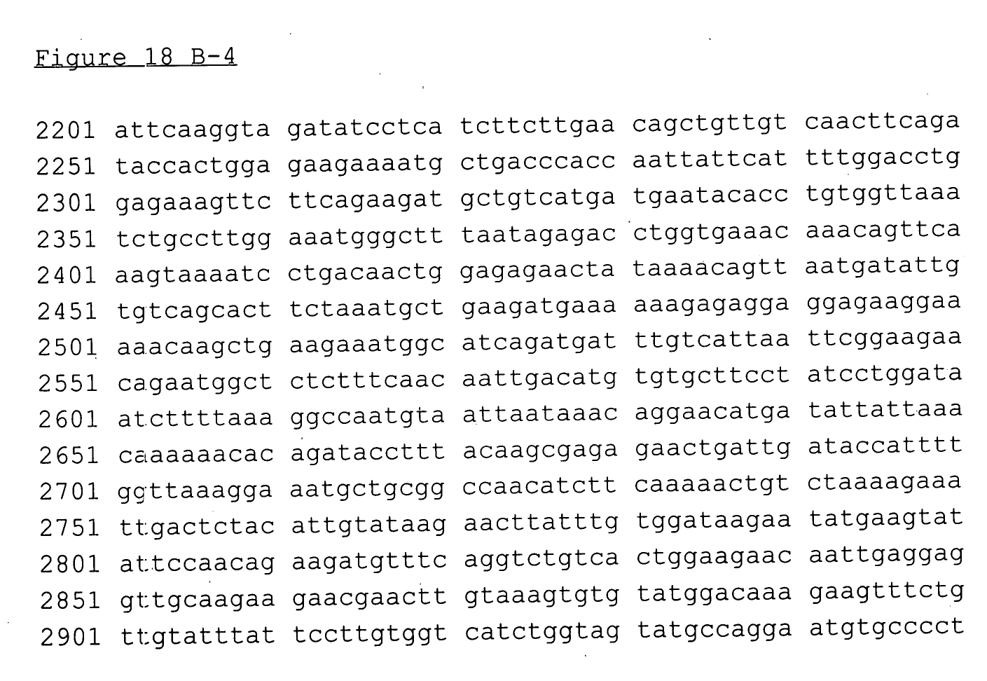

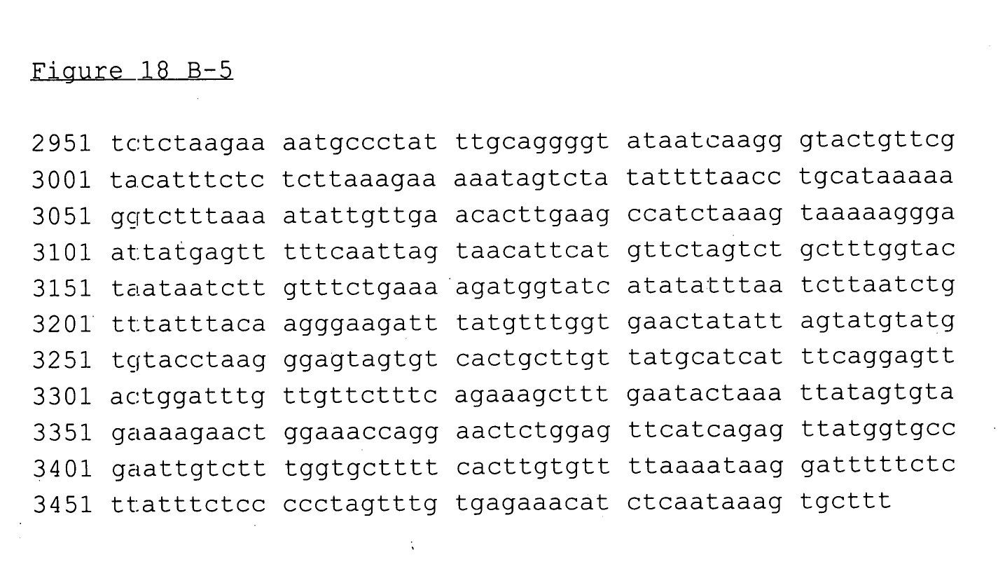

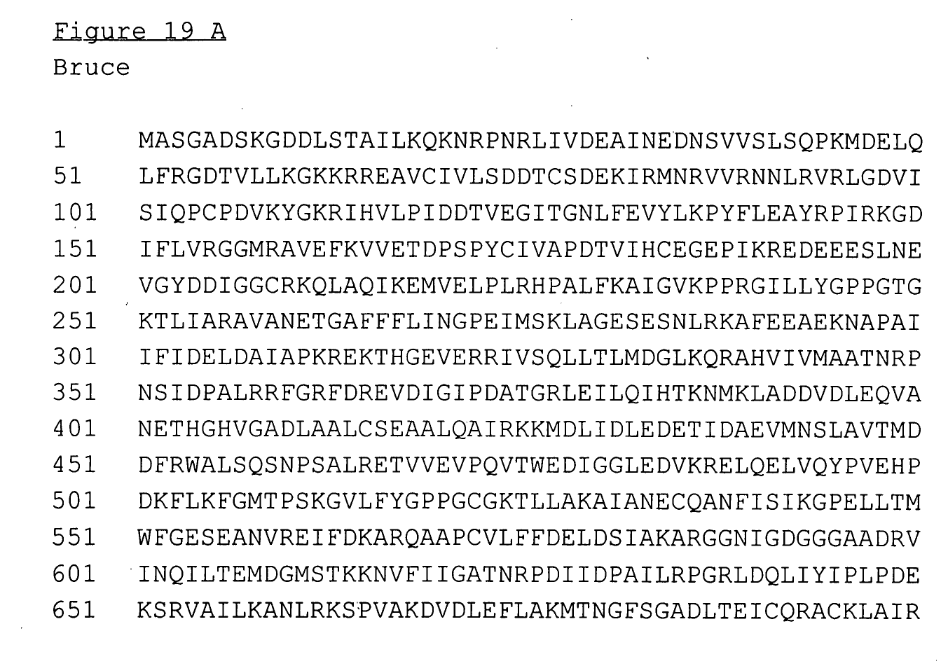

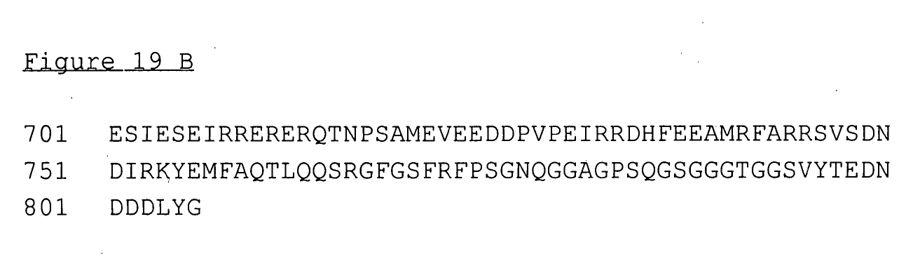

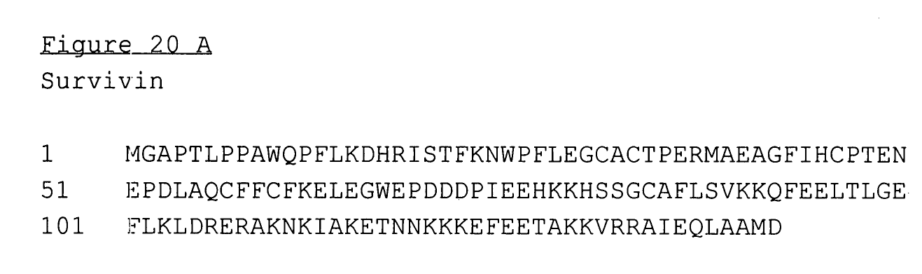

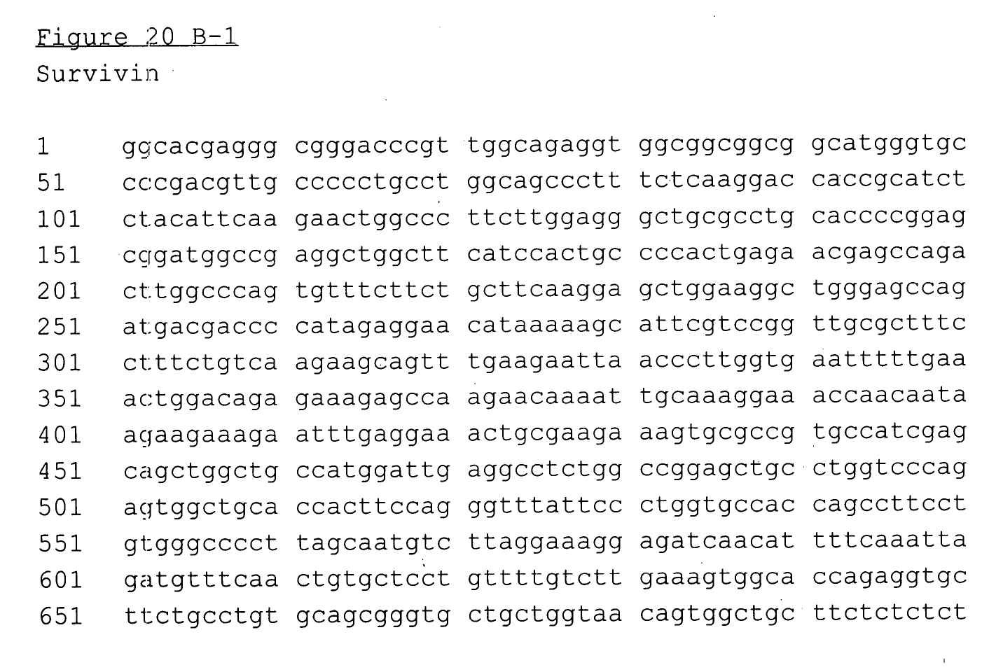

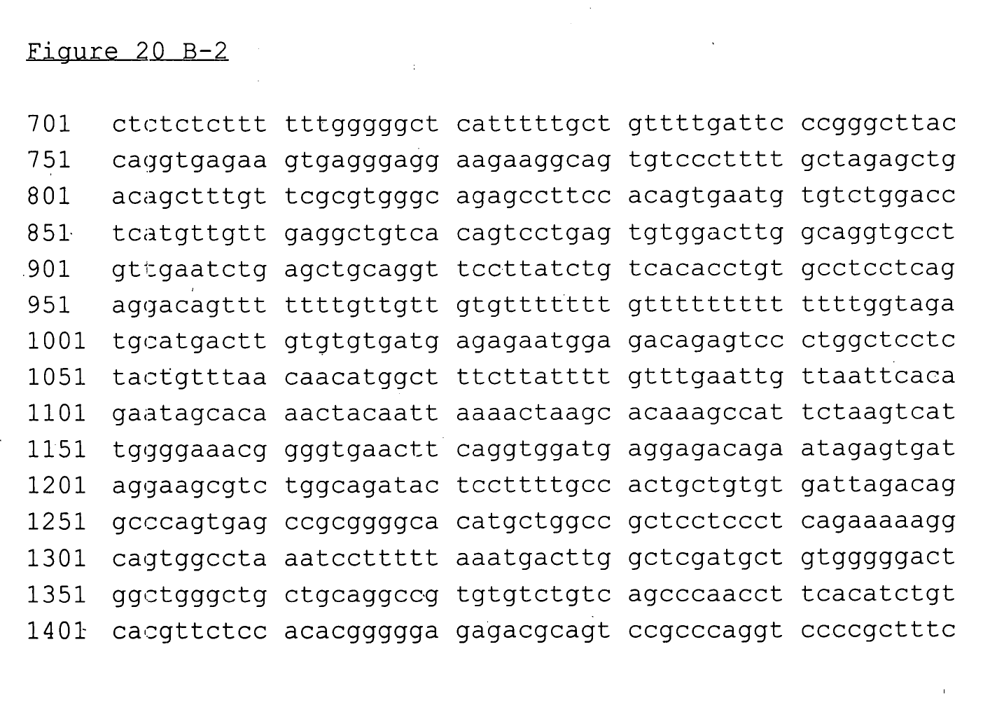

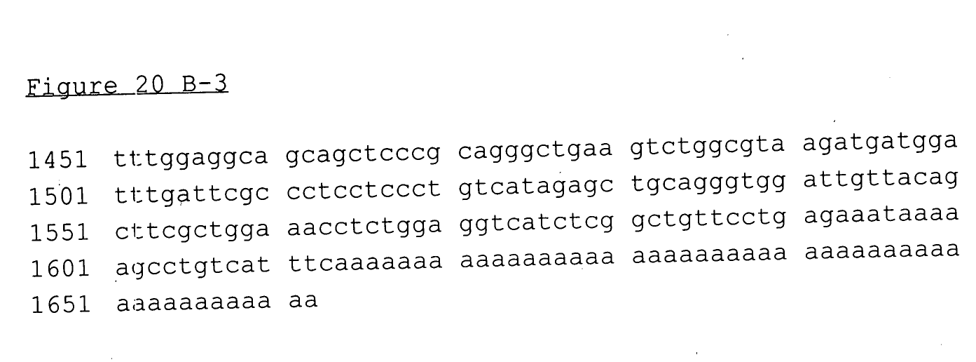

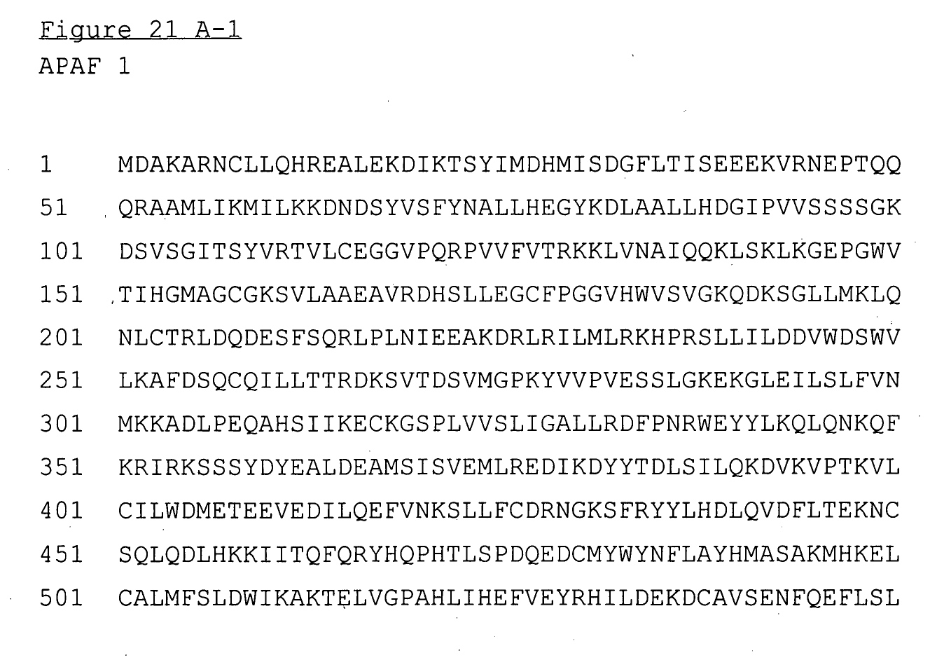

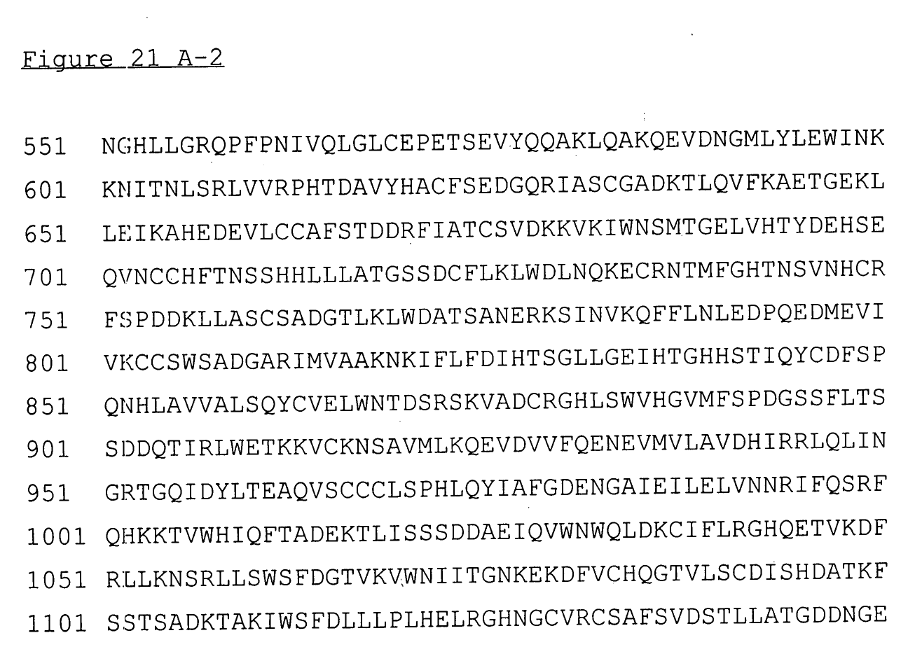

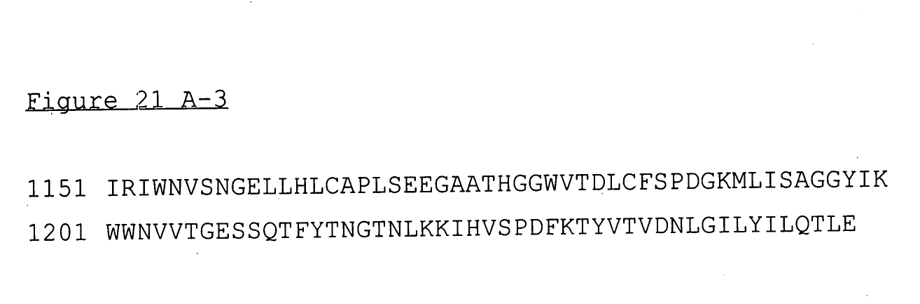

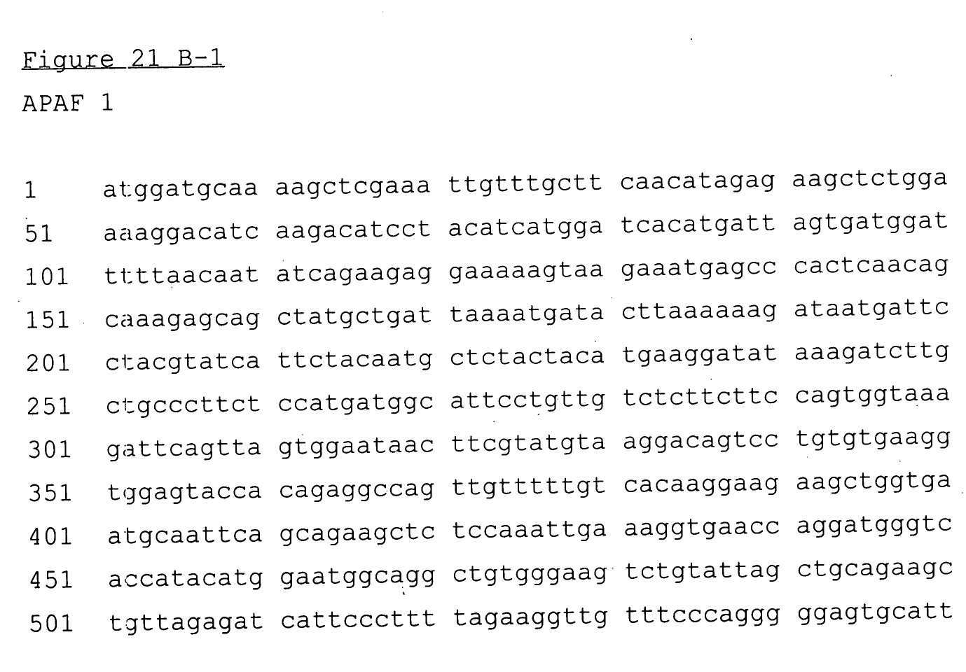

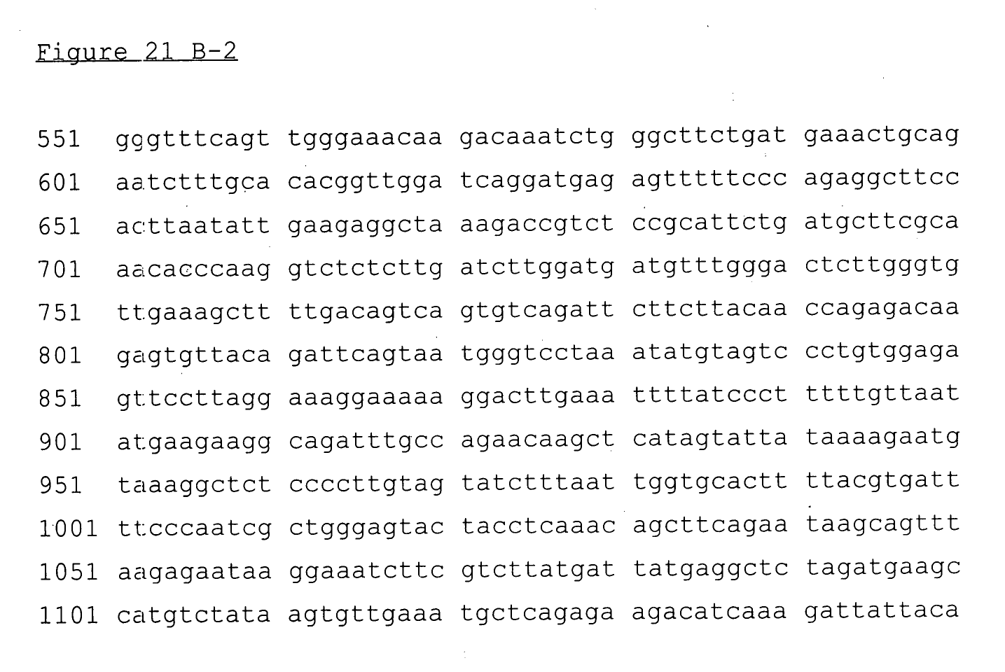

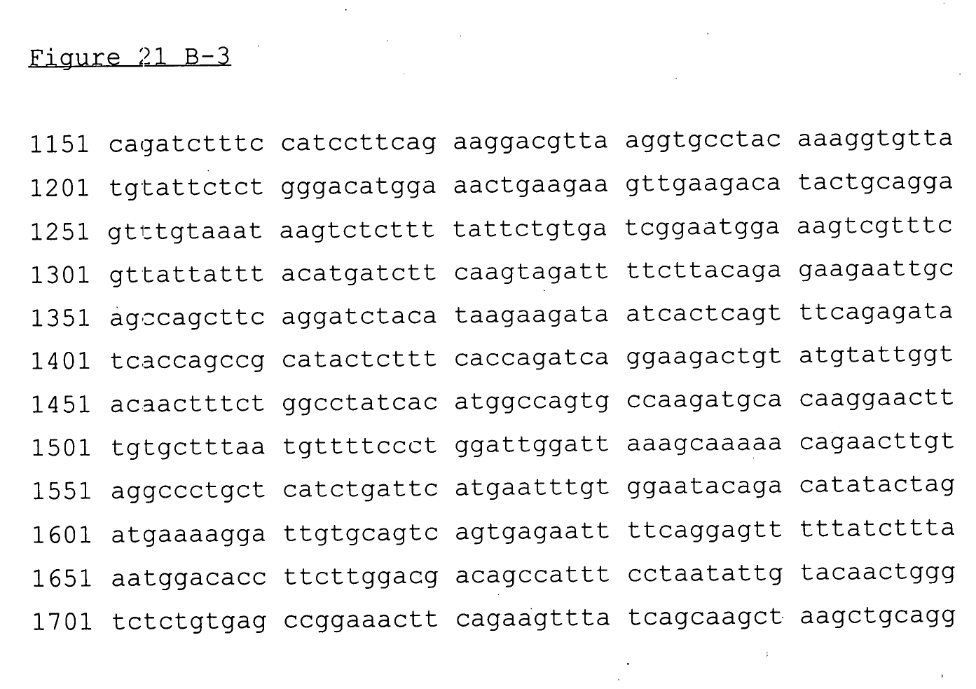

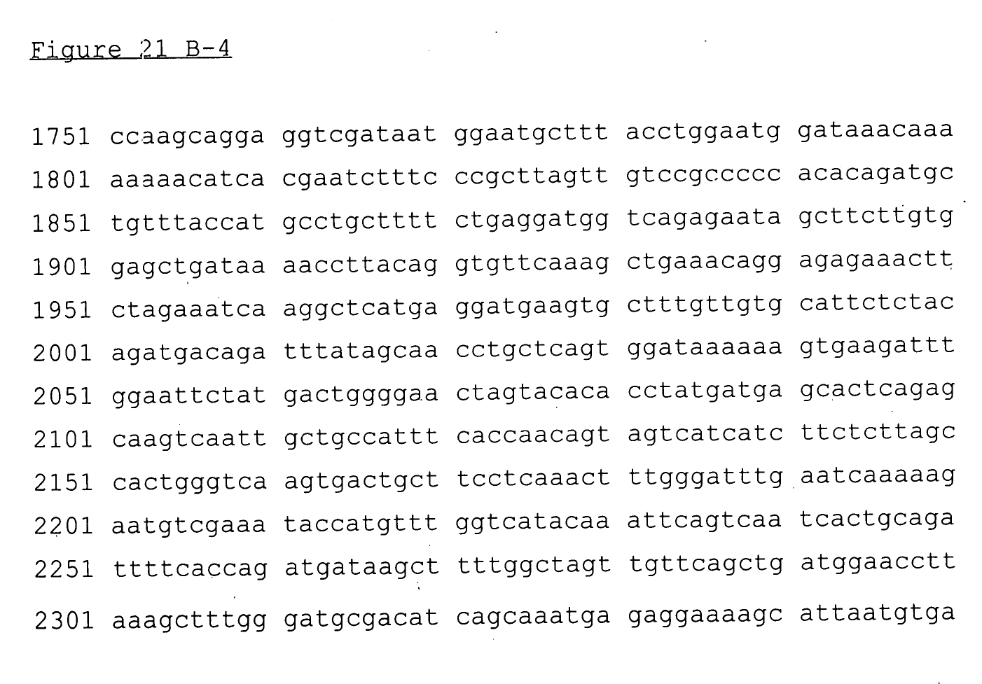

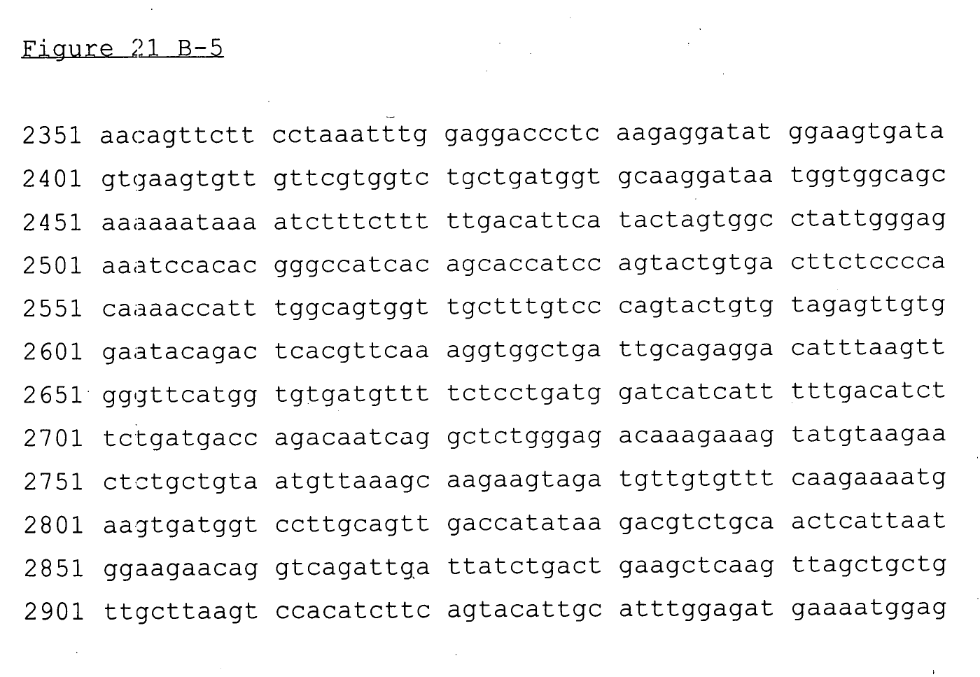

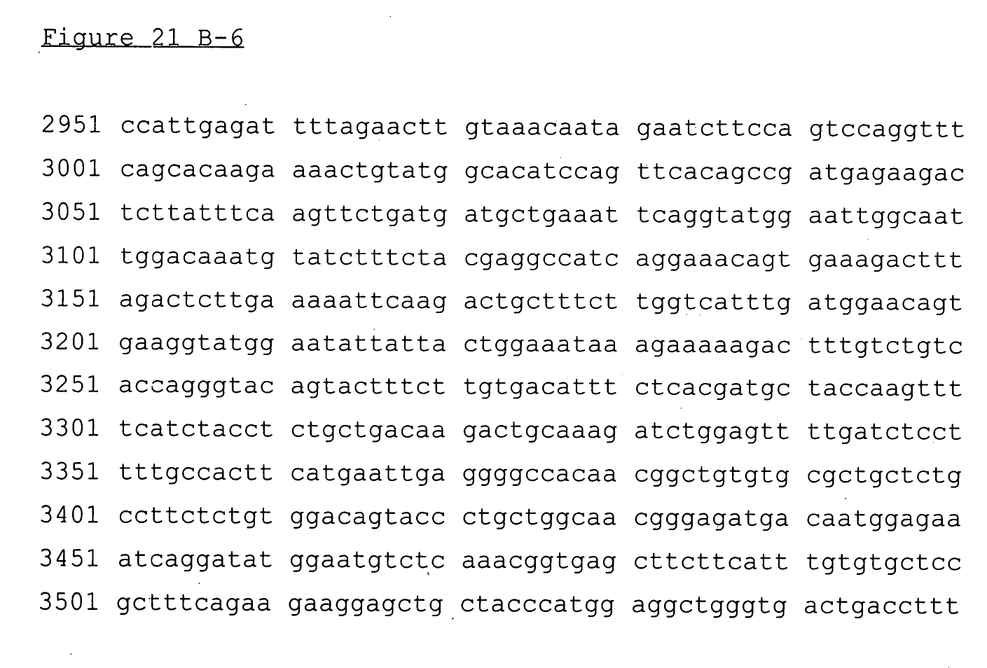

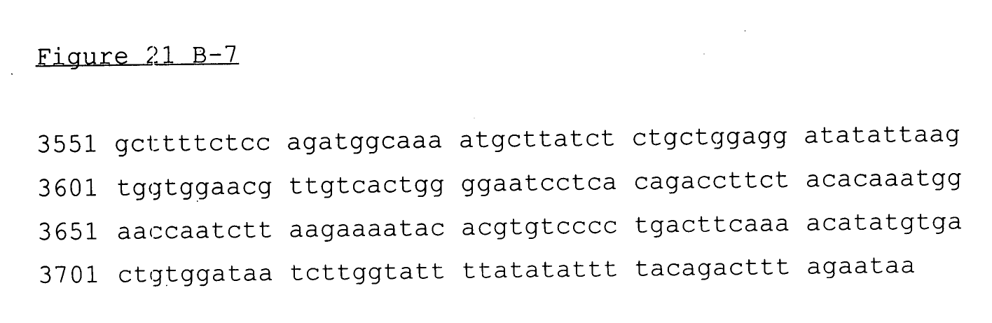

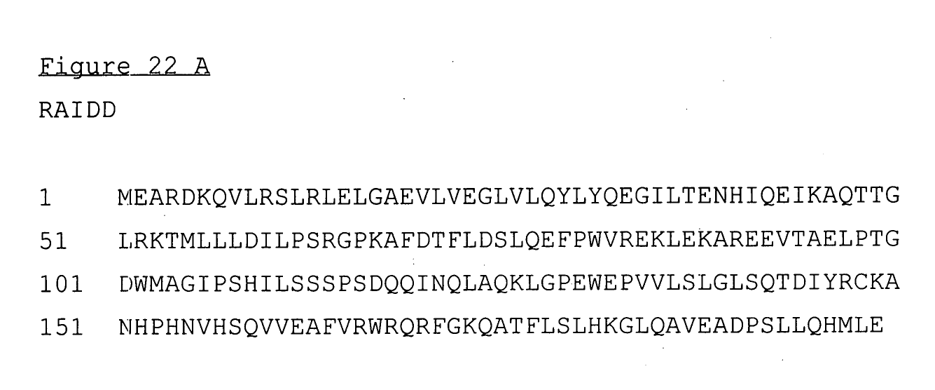

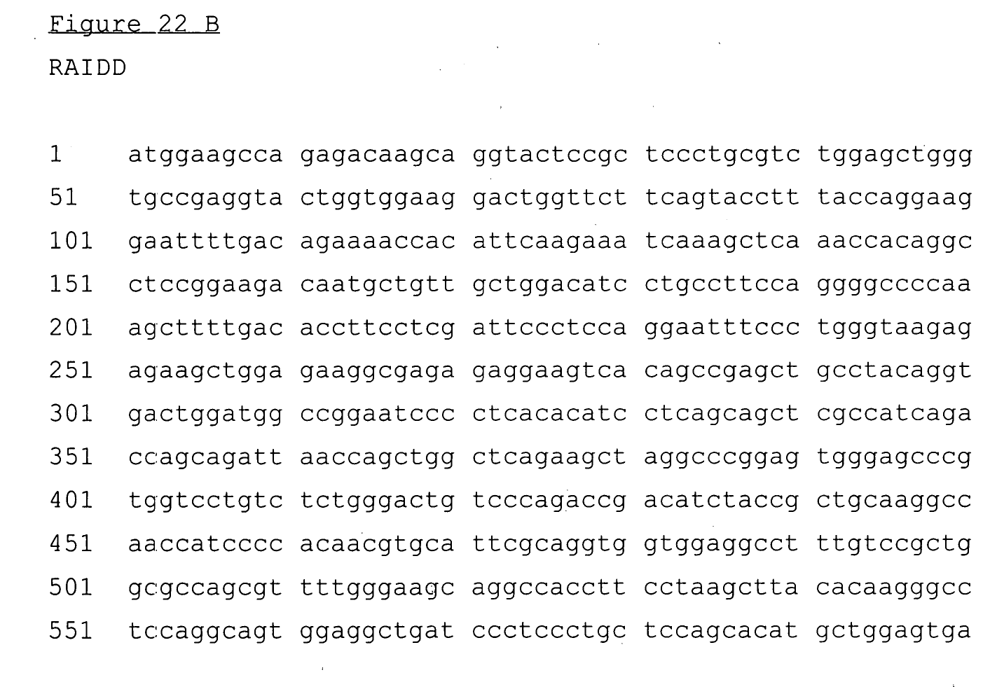

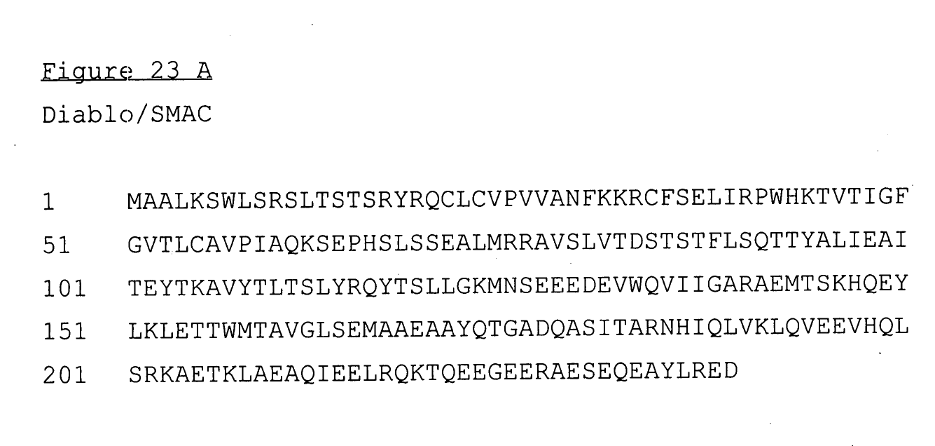

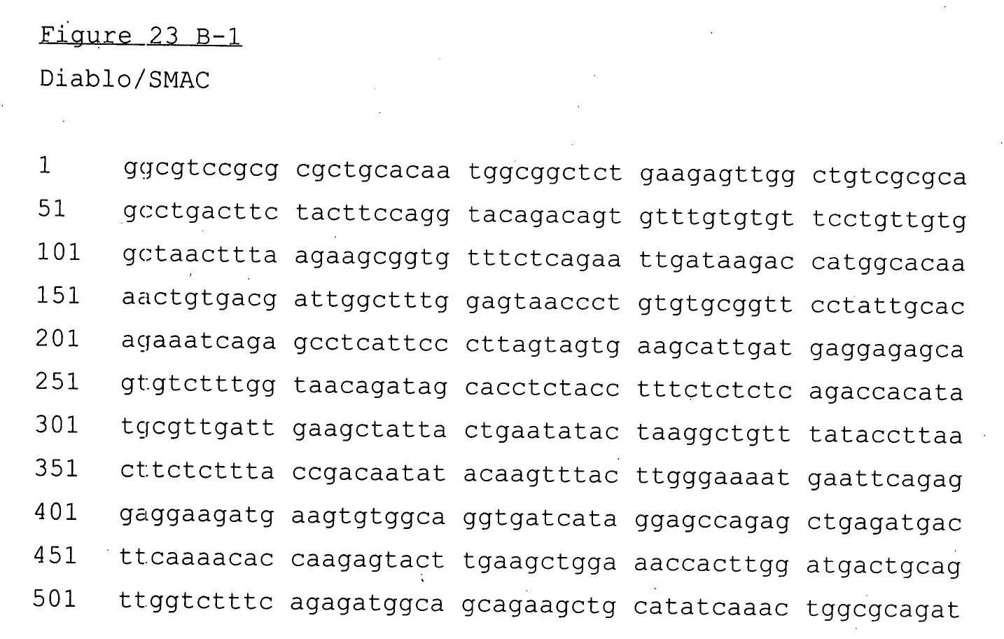

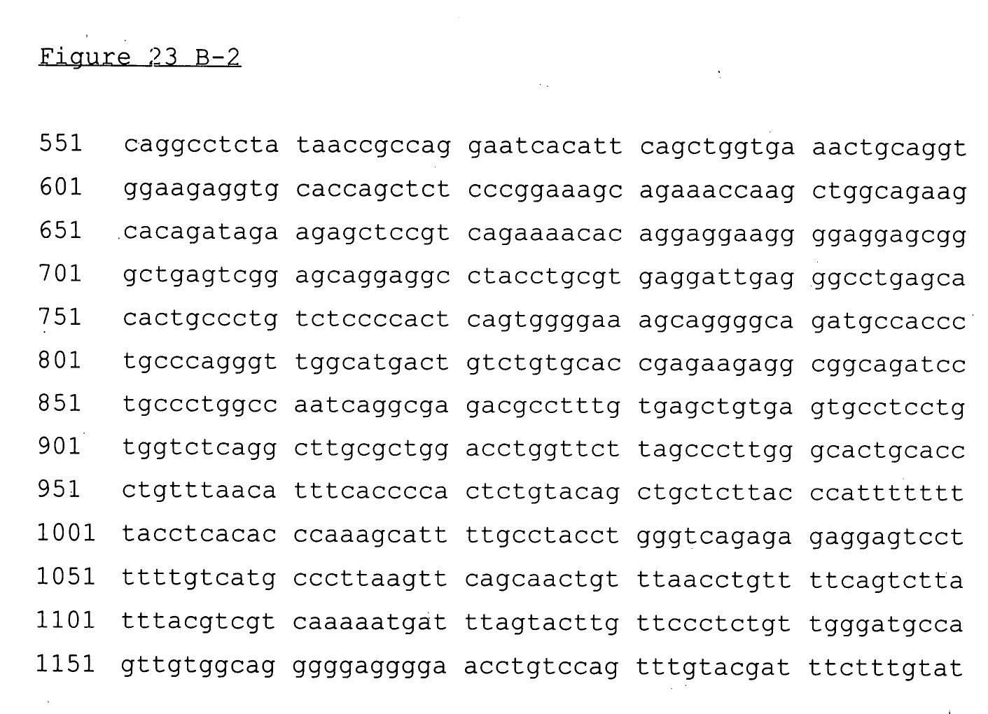

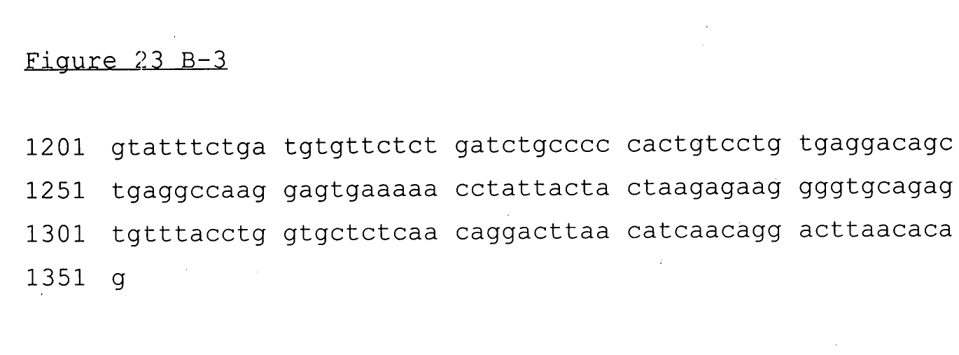

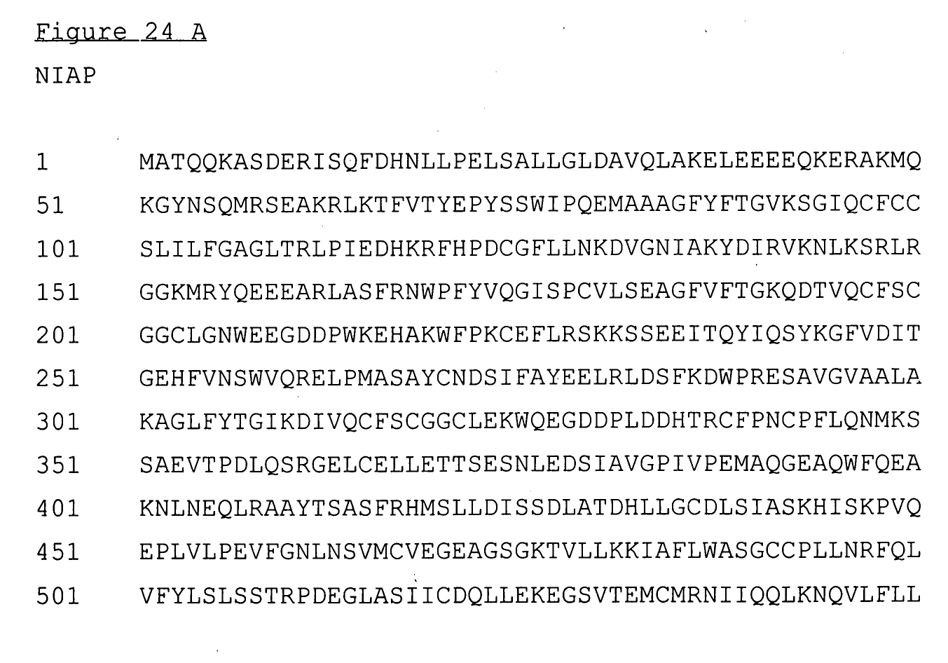

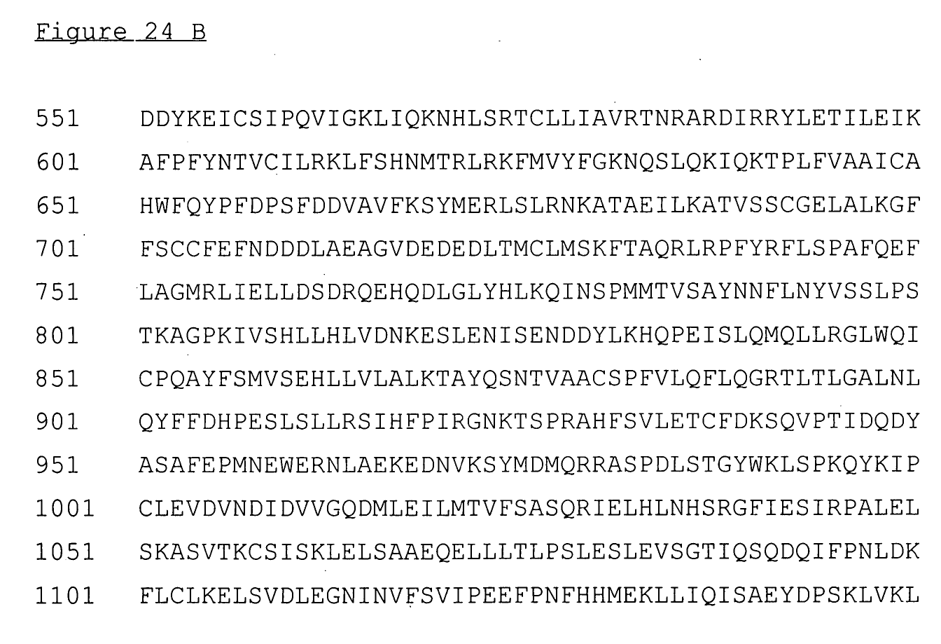

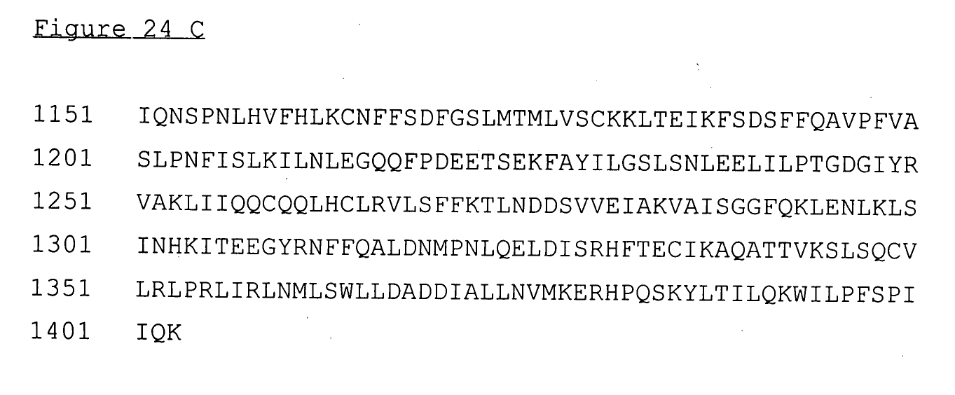

Differential inhibition of TFD-induced death by DEVD-FMK in wild-type and caspase-2-null neurons. Sympathetic neurons from wild-type (FIG. 1A) and caspase-2-null (FIG. 1B) mice were cultured for five days and then washed and treated with anti-NGF in the presence and absence of BAF (50 μM) or DEVD-FMK (10 μM). Cultures were counted daily and survival is reported relative to that in the same cultures before NGF deprivation and is given as mean±SEM (n=3). Error bars are sometimes too small to be visible. This is a representative experiment. Similar results were obtained in four independent experiments.

FIG. 2A

FIG. 2A, like FIGS. 2B-2E, shows differential expression of caspases and caspase regulatory molecules in wild-type and caspase-2-null mice. Relative expression of caspase mRNAs in wild-type and caspase-2-null P1 mouse brains. mRNA was prepared from 6 wild-type and 9 caspase-2-null mouse brains. cDNA from each brain was analyzed individually, using serial dilutions in duplicate, and using real-time PCR. Each sample was analyzed three times. Results were normalized to actin mRNA levels. For each caspase, expression in wild-type brains was set at a value of 1. Data are the means±SEM (n=6 for wild-type, n=9 for caspase-2-null).

FIG. 2B

Western blots for caspase-9 and caspase-3. Wild-type and caspase-2-null P1 mouse brains were homogenized in sample buffer and equal amounts of protein (determined by the Bradford protein assay) were subjected to Western blotting using the indicated antisera. Actin staining confirmed equal loading. These are representative blots. Similar results were obtained in 6 independent blots for caspase-9, 3 independent blots for caspase-3.

FIG. 2C

Relative expression of Diablo, APAF-1, RAIDD and MIAP3 mRNA in wild-type and caspase-2-null mouse brains. mRNA from 6 wild-type and 9 caspase-2 mouse brains was analyzed using real-time PCR as described in FIG. 2A. Results were normalized to actin mRNA levels. For each transcript, results in wild-type are set at a value of 1. Data are the means±SEM (n=6 for wild-type, n=9 for caspase-2-null).

FIG. 2D

Western blots for DIABLO/Smac (also referred to herein as Diablo/SMAC), APAF-1 (also referred to herein as Apaf-1), MIAP3. Wild-type and caspase-2-null P1 mouse brains were homogenized in sample buffer and equal amounts of protein were subjected to Western blotting using the indicated antisera. Actin staining confirmed equal loading. These are representative blots. Similar results were obtained with 6 independent blots for DIABLO/Smac, 3 independent blots for APAF-1 and MIAP3.

FIG. 2E

Relative expression of caspase, DIABLO/Smac, and MIAP3 mRNAs in wild-type and caspase-2-null P1 cultured sympathetic neurons. mRNA was prepared from wild-type and caspase-2-null sympatheic neurons grown in culture for 6 days. cDNA was analyzed with real-time PCR using serial dilutions in duplicate. Each sample was analyzed three times. Results were normalized to actin mRNA levels. For each mRNA, expression in wild-type brains was set at a value of 1. Data are the means±SEM (n=3 for wild-type, n=3 for caspase-2-null).

FIG. 3A

FIG. 3A, like FIGS. 3B and 3C, shows differential effects of down-regulation of specific caspases on TFD-induced death of wild-type and caspase-2-null sympathetic neurons. PENETRATIN1™-linked antisense oligonucleotides specifically down-regulate targeted caspases. PC12 cells were treated with the indicated antisense oligonucleotides (240 nM) for 6 hours. Cell lysates containing equal amounts of protein were subjected to Western blotting using the corresponding antisera. Actin staining confirmed equal loading. These are representative blots. Similar results were obtained in 2 independent experiments. Sympathetic neurons from P1 wild-type (FIG. 3B) and caspase-2-null (FIG. 3C) mice were cultured for 5 days. Cultures were then washed and treated with anti-NGF in the presence and absence of the indicated antisense oligonucleotides (see legend at right). Cultures were scored daily, and survival is reported relative to the numbers of living neurons in the same cultures before NGF deprivation and is given as mean±SEM (n=3). Error bars are sometimes too small to be visible. This is a representative experiment. Similar results were obtained in 6 independent experiments.

FIGS. 4A-4E

Caspase-2-null neurons employ a pathway alternative to TFD-induced death. Sympathetic neurons from P1 wild-type (FIGS. 4A, 4D) and caspase-2-null (FIGS. 4B, 4E) mice were cultured for 5 days. Cultures were then washed and treated with anti-NGF in the presence and absence of V-ADiablo (FIGS. 4A, 4B) or V-AAPAF-1 (FIGS. 4D, 4E). Cultures were scored daily, and survival is reported relative to that in the same cultures before NGF deprivation and is given as mean±SEM (n=3). Error bars are sometimes too small to be visible. This is a representative experiment. Similar results were obtained in 3 independent experiments. Specific down-regulation of DIABLO/Smac and APAF-1 by antisense oligonucleotides is shown in FIG. 4C. PC12 cells were treated with the indicated antisense oligonucleotides (240 nM) for 6 hours. Cells lysates containing equal amounts of protein were subjected to Western blotting using the corresponding antisera. Actin staining confirmed equal loading. These are representative blots. Similar results were obtained in 2 independent experiments.

FIGS. 5A-5F

Photomicrographs of SCGs from caspase-2-null mice rescued by down-regulation of various components of the caspase-9 pathway are shown. Sympathetic neurons for caspase-2-null mice were cultured for 5 days. Cultures were then washed and treated with anti-NGF in the presence or absence of various antisense oligonucleotides. The photomicrographs were taken after two days of treatment. FIG. 5A: +NGF; FIG. 5B: Anti-NGF; FIG. 5C: Anti-NGF+V-ADiablo; FIG. 5D: Anti-NGF+V-AAPAF-1; FIG. 5E: Anti-NGF+V-ACasp9; and FIG. 5F: Anti-NGF+V-ACasp3. Bar=100 μm.

FIGS. 6A-6F

Down-regulation of various components of the caspase-9 pathway suppresses caspase-3 activation in NGF-deprived sympathetic neurons from caspase-2-null mice. Sympathetic neurons from caspase-2-null mice were cultured on chamber coverglass slides for 5 days. Cultures were then washed and treated with anti-NGF in the presence and absence of various oligonuculeotides. After 5 hours, cells were fixed, immunostained for actin and activated caspase-3 and examined by confocal microscopy (colors not shown). FIG. 6A: +NGF; FIG. 6B: Anti-NGF; FIG. 6C: Anti-NGF+V-ADiablo; FIG. 6D: Anti-NGF+V-AAPAF-1; FIG. 6E: Anti-NGF+V-ACasp9; and FIG. 6F: Anti-NGF+V-ACasp3. Bar=50 μm.

FIG. 7A

FIG. 7A, like FIGS. 7B-7E, show that down-regulation of MIAP3 permits caspase-9-dependent TFD-induced death of wild-type SCGs. FIG. 7A shows specific down-regulation of MIAP3. PC12 cells were treated with V-AMIAP3 (240 nM) for 6 hours. Cells lysates containing equal amounts of protein were subjected to Western blotting using the corresponding antisera. Actin staining confirmed equal loading. These are representative blots. Similar results were obtained in 2 independent experiments.

FIG. 7B

Sympathetic neurons from wild-type mice were cultured for 5 days. Cultures-were then washed and treated with anti-NGF in the presence and absence of the indicated antisense oligonucleotides. Cultures were scored daily, and survival is reported relative to that in the same cultures before NGF deprivation and is given as mean±SEM (n=3). Error bars are sometimes too small to be visible. This is a representative experiment. Similar results were obtained in 3 independent experiments.

FIGS. 7C-7E

Activation of caspase-3 in NGF-deprived sympathetic neurons is dependent on caspase-9. Sympathetic neurons from wild-type mice were cultured on chamber coverglass slides for 5 days.

Cultures were then washed and treated with anti-NGF in the presence and absence of various oligonucleotides. After 5 hours, cells were fixed, immunostained for actin and activated caspase-3 and examined with confocal microscopy (color not shown). FIG. 7C:-NGF; FIG. 7D: Anti-NGF+V-ACasp2+V-AMIAP3; and FIG. 7E: Anti-NGF+V-ACasp2+V-AMIAP3+V-ACasp9. Bar=50 μm.

FIGS. 8A and 8B

Schematic representation of the trophic factor deprivation death pathways in sympathetic neurons.

FIG. 9

Relative expression of MIAP mRNAs in wild-type and caspase-2-null P1 cultured sympathetic neurons. mRNA was prepared from wild-type and caspase-2-null sympathetic neurons grown in culture for 6 days. cDNA was analyzed with real-time PCR using serial dilutions in duplicate.

FIG. 10

Aβ activation of the caspase-8 pathway is suppressed by MIAP2 in sympathetic neurons. Wild-type sympathetic neuron cultures were treated with aggregated β-amyloid in the presence or absence of the indicated antisense oligonucleotides for 2 days. Cultures were scored daily, and survival is reported relative to the numbers of living neurons in the cultures before NGF deprivation and is given as mean±SEM (n=3).

FIG. 11

Aβ death in caspase-2-null SCGs can utilize the caspase-8 pathway when MIAP2 is suppressed. Caspase-2-null sympathetic neuronal cultures were treated with Aβ in the presence or absence of the indicated antisense oligonucleotides for 2 days. Cultures were scored daily, and survival is reported relative to the numbers of living neurons in the same cultures before NGF deprivation and is given as a mean±SEM (n=3).

FIG. 12

Aβ activation of the caspase-8 pathway is suppressed by MIAP2 in hippocampal neurons. Cultured hippocampal neurons were treated with Aβ in the presence or absence of the indicated oligonucleotides. Neuronal survival was determined after one day of treatment and is given as a mean±SEM (n=3).

FIG. 13

This Figure sets forth the (a) amino acid sequence of, and (b) nucleotide sequence encoding, MIAP1.

FIG. 14

This Figure sets forth the (a) amino acid sequence of, and (b) nucleotide sequence encoding, MIAP2.

FIG. 15

This Figure sets forth the (a) amino acid sequence of, and (b) nucleotide sequence encoding, MIAP3.

FIG. 16

This Figure sets forth the (a) amino acid sequence of, and (b) nucleotide sequence encoding, CIAP1.

FIG. 17

This Figure sets forth the (a) amino acid sequence of, and (b) nucleotide sequence encoding, CIAP2.

FIG. 18

This Figure sets forth the (a) amino acid sequence of, and (b) nucleotide sequence encoding, XIAP.

FIG. 19

This Figure sets forth the amino acid sequence of human Bruce.

FIG. 20

This Figure sets forth the (a) amino acid sequence of, and (b) nucleotide sequence encoding, human Survivin.

FIG. 21

This Figure sets forth the (a) amino acid sequence of, and (b) nucleotide sequence encoding, human APAF-1.

FIG. 22

This Figure sets forth the (a) amino acid sequence of, and (b) nucleotide sequence encoding, human RAIDD.

FIG. 23

This Figure sets forth the (a) amino acid sequence of, and (b) nucleotide sequence encoding, human Diablo/SMAC.

FIG. 24

This Figure sets forth the amino acid sequence of human NAIP.

### DETAILED DESCRIPTION OF THE INVENTION

Definitions

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Antisense nucleic acid” shall mean any nucleic acid which, when introduced into a cell, specifically hybridizes to at least a portion of an mRNA in the cell encoding a protein (“target protein”) whose expression is to be inhibited, and thereby inhibits the target protein's expression. The instant nucleic acids are antisense nucleic acids, and can hybridize to an mRNA at its protein-coding region and/or its non-coding region (e.g., 5′-untraslated region). Hybridization can also occur at an mRNA splice site, ribosome-binding site, and/or at or near the initiation codon (e.g., from just upstream of the initiation codon to about 10 nucleotides downstream therefrom).

“Nucleic acid” shall mean any nucleic acid molecule, including, without limitation, DNA, RNA and hybrids thereof. Nucleic acids include, for example, oligonucleotides. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Specifically hybridize” to a nucleic acid shall mean, with respect to a first nucleic acid, that the first nucleic acid hybridizes to a second nucleic acid with greater affinity than to any other nucleic acid.

“Subject” shall mean any animal, such as a human, a non-human primate, a mouse, a rat, a guinea pig or a rabbit.

“Treating” a disorder shall mean slowing, stopping or reversing the disorder's progression. In the preferred embodiment, treating a disorder means reversing the disorder's progression, ideally to the point of eliminating the disorder itself.

Embodiments of the Invention

This invention provides a first nucleic acid which specifically hybridizes to a nucleic acid encoding an inhibitor-of-apoptosis protein.

In one embodiment, the nucleic acid is complementary to the nucleic acid encoding the inhibitor-of-apoptosis protein. In another embodiment, the nucleic acid is an oligonucleotide having a length of from about 15 nucleotides to about 25 nucleotides. Specifically envisioned is an oligonucleotide having a length of 15, 16, 17, 18, 19, 20, 21, 22, 23, 24 or 25 nucleotides. Also envisioned is an oligonucleotide that hybridizes to at least eight consecutive nucleotides, and oligonucleotides of at least about 15 nucleotides in length.

With respect to the first nucleic acid, the inhibitor-of-apoptosis protein can be any inhibitor-of apoptosis protein including, without limitation, MIAP1 (FIG. 13, GenBank Accession No. NM_{—}007464), MIAP2 (FIG. 14, GenBank Accession No. NM_{—}007465), MIAP3 (FIG. 15, GenBank Accession No. NM009688), CIAP1 (FIG. 16, GenBank Accession No. XM006266), CIAP2 (FIG. 17, GenBank Accession No. XM006267), XIAP (FIG. 18, GenBank Accession No. NM001166, U.S. Pat. Nos. 6,171,821 and 6,159,709), Bruce (FIG. 19), Survivin (FIG. 20, GenBank Accession No. BC008718), NAIP (FIG. 24), ML-IAP and ILP2. In one embodiment, the first nucleic acid specifically hybridizes to the portion of the nucleic acid encoding MIAP3 beginning with the adenosine at position 769 and ending with the guanosine at position 791.

This invention also provides a first composition comprising the first nucleic acid and a carrier.

In one embodiment, the first composition comprises nucleic acids which specifically hybridize to nucleic acids encoding a plurality of inhibitor-of-apoptosis proteins. Such pluralities of inhibitor-of-apoptosis proteins include, without limitation, (a) CIAP1, CIAP2 and XIAP; (b) CIAP1 and XIAP; (c) CIAP2 and XIAP; and (d) CIAP1 and CIAP2.

In one embodiment of the first composition, the carrier comprises a diluent, an adjuvant, a virus, a liposome, a microencapsule, a neuronal cell receptor ligand, a neuronal-specific virus, a polymer-encapsulated cell or a retroviral vector. In another embodiment, the carrier is an aerosol, an intravenous carrier, an oral carrier or a topical carrier.

This invention further provides a method for inducing a cell's death which comprises contacting the cell with the first nucleic acid under conditions permitting the nucleic acid to enter the cell.

In one embodiment, this method further comprises contacting the cell with nucleic acids which specifically hybridize to nucleic acids encoding a plurality of inhibitor-of-apoptosis proteins. Each plurality of inhibitor-of-apoptosis proteins includes, without limitation, (a) CIAP1, CIAP2 and XIAP; (b) CIAP1 and XIAP; (c) CIAP2 and XIAP; and (d) CIAP1 and CIAP2.

In one embodiment of this method, the conditions permitting the nucleic acid to enter the cell comprise the use of a vector, a liposome, a mechanical means or an electrical means. Such vectors include, without limitation, a plasmid, a cosmid, a bacterophage vector, an adenovirus vector, an adeno-associated virus vector, a protein vector (e.g., PENETRATIN1™), an Epstein-Barr virus vector, a Herpes virus vector, an LXSN vector, an LNL6 vector, an attenuated HIV vector (e.g., TAT), a retroviral vector (e.g., MMuLV vector) and a vaccinia virus vector. Such liposomes include, for example, antibody-coated liposomes.

This invention further provides a method for treating a subject afflicted with cancer which comprises administering to the subject a therapeutically effective amount of the first nucleic acid.

Cancers treated by this method include, without limitation, acute lymphocytic leukemia, acute myelogenous leukemia, lung cancer, breast cancer, ovarian cancer, prostate cancer, lymphoma, Hodgkin's disease, malignant melanoma, neuroblastoma, renal cell carcinoma and squamous cell carcinoma. In one embodiment, the cancer is a tumor.

This method can be applied to any subject. In one embodiment, the subject is a mammal. Preferably, the subject is a human.

This invention provides a second nucleic acid that specifically hybridizes to a nucleic acid which encodes a protein, other than caspase-2, that induces cell death.

In one embodiment, the nucleic acid is complementary to the nucleic acid encoding the protein that induces cell death. In another embodiment, the nucleic acid is an oligonucleotide having a length of from about 15 nucleotides to about 25 nucleotides. Specifically envisioned is an oligonucleotide having a length of 15, 16, 17, 18, 19, 20, 21, 22, 23, 24 or 25 nucleotides. Also envisioned is an oligonucleotide that hybridizes to at least eight consecutive nucleotides, and oligonucleotides of at least about 15 nucleotides in length.

With respect to the second nucleic acid, the cell death-inducing protein can be any such protein including, without limitation, APAF1 (FIG. 21, GenBank Accession No. AF149794), RAIDD (FIG. 22, GenBank Accession No. U87229, U.S. Pat. No. 6,130,079), Diablo/SMAC (FIG. 23, GenBank Accession No. XM006685), and Htr2/Omi.

In one embodiment, the second nucleic acid which specifically hybridizes to a nucleic acid encoding the protein APAF1 having the amino acid sequence shown in FIG. 21, and in a further embodiment, specifically hybridizes to the portion of the nucleic acid encoding APAF-1 beginning with the cytosine at position 576 and ending with the adenosine at position 596.

In another embodiment, the second nucleic acid specifically hybridizes to a nucleic acid encoding the protein RAIDD having the amino acid sequence shown in FIG. 22, and in a further embodiment, specifically hybridizes to the portion of the nucleic acid encoding RAIDD beginning with the guanosine at position 110 and ending with the adenosine at position 130.

In another embodiment, the second nucleic acid specifically hybridizes to a nucleic acid encoding the protein Diablo/SMAC having the amino acid sequence shown in FIG. 23, and in a further embodiment, specifically hybridizes to the portion of the nucleic acid encoding Diablo/SMAC beginning with the thymidine at position 1 and ending with the thymidine at position 21.

This invention provides a second composition comprising the second nucleic acid and a carrier. In one embodiment, the second composition comprises nucleic acids which specifically hybridize to nucleic acids encoding a plurality of proteins that induce cell death. Such pluralities include, without limiation, (a) APAF-1 and Diablo/SMAC; (b) APAF-1, Diablo/SMAC and caspase-9; (c) APAF-1, Diablo/SMAC and caspase-7; (d) caspase-2 and RAIDD; (e) caspase-8 and RAIDD; (f) caspase-8, RAIDD and caspase-3; and (g) caspase-2 and caspase-9.

In one embodiment of the second composition, the carrier comprises a diluent, an adjuvant, a virus, a liposome, a microencapsule, a neuronal cell receptor ligand, a neuronal-specific virus, a polymer-encapsulated cell or a retroviral vector. In another embodiment, the carrier is an aerosol, an intravenous carrier, an oral carrier or a topical carrier.

This invention further provides a method for inhibiting a cell's death which comprises contacting the cell with the second nucleic acid under conditions permitting the nucleic acid to enter the cell.

This invention still further provides a method for inhibiting a neuronal cell's death which comprises contacting the cell with the second nucleic acid under conditions permitting the nucleic acid to enter the cell.

In one embodiment, these methods further comprise contacting the cell with nucleic acids which specifically hybridize to nucleic acids encoding a plurality of proteins that induce cell death. Such pluralities include, without limitation, (a) APAF-1 and Diablo/SMAC; (b) APAF-1, Diablo/SMAC and caspase-9; (c) APAF-1, Diablo/SMAC and caspase-7; (d) caspase-2 and RAIDD; (e) caspase-8 and RAIDD; (f) caspase-8, RAIDD and caspase-3; and (g) caspase-2 and caspase-9.

In one embodiment of the instant methods, the conditions permitting the second nucleic acid to enter the cell comprise the use of a vector, a liposome, a mechanical means or an electrical means. Such vectors include, without limitation, a plasmid, a cosmid, a bacterophage vector, an adenovirus vector, an adeno-associated virus vector, a protein vector (e.g., PENETRATIN1™), an Epstein-Barr virus vector, a Herpes virus vector, an LXSN vector, an LNL6 vector, an attenuated HIV vector (e.g., TAT), a retroviral vector (e.g. MMuLV vector) and a vaccinia virus vector. Such liposomes include, for example, antibody-coated liposomes.

This invention further provides a method for treating a neurodegenerative disorder in a subject which comprises administering to the subject a therapeutically effective amount of the second nucleic acid. Neurodegenerative disorders include, for example, brain disorders and central nervous system disorders.

Finally, this invention provides a method for treating a heart disorder in a subject which comprises administering to the subject a therapeutically effective amount of the second nucleic acid. Heart disorders include, for example, cardiomyopathy.

The methods employing the second nucleic acid can be applied to any subject. In one embodiment, the subject is a mammal. Preferably, the subject is a human.

In this invention, nucleic acid sequences encoding certain inhibitor-of-apoptosis proteins and proteins that induce cell death, as well as the amino acid sequences thereof, are set forth herein as follows: MIAP1 (FIG. 13), MIAP2 (FIG. 14), MIAP3 (FIG. 15), CIAP1 (FIG. 16), CIAP2 (FIG. 17), XIAP (FIG. 18), Bruce (FIG. 19), Survivin (FIG. 20), APAF1 (FIG. 21), RAIDD (FIG. 22), and Diablo/SMAC (FIG. 23).

Determining a therapeutically effective amount of the instant nucleic acids can be done based on animal data using routine computational methods. In one embodiment, the therapeutically effective amount contains between about 0.1 ug and about 1 g of nucleic acid. In other embodiments, the effective amount contains between (a) about 1 ug and about 100 mg of nucleic acid, (b) about 10 ug and about 10 mg of the nucleic acid, (c) about 100 ug and about 1 mg of the nucleic acid, (d) about 1 mg and about 100 mg of the nucleic acid, and (e) about 10 mg and about 50 mg of the nucleic acid. Additionally, the nucleic acid can be administered to the subject, for example, one time only, once in a 24-hour period, more than once in a 24-hour period, and for more than one day.

In this invention, administering the instant nucleic acids can be effected or performed using any of the various methods and delivery systems known to those skilled in the art. The administering can be performed, for example, intravenously, orally, nasally, via ocular, anal or otic delivery, via implant, via liposome, via viral infection (e.g., via non-integrating, replication-defective virus), via gene bombardment, transmucosally, transdermally, intramuscularly, and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

This invention will be better understood from the Experimental Details which follow. However, one skilled in the art will readily appreciate that the specific methods and results discussed are merely illustrative of the invention as described more fully in the claims which follow thereafter.

Experimental Details

Evidence suggests that caspases are activated in cascades where upstream (activator) caspases lead to activation of downstream (effector) caspases. One of the most studied cascades is that of caspase-9 which leads to activation of caspases-3 and-7. Activation of the caspase-9 dependent apoptotic pathway is tightly regulated by both the regulatory adaptor molecule Apaf-1, which recruits caspase-9 to the apopotosome, and by the inhibitors of apoptosis proteins (Salvesen 1999; Hengartner 2000). The human IAP, XIAP, has been shown to inhibit caspase-9 as well as the downstream caspases, caspases-3 and -7 (Deveraux et al. 1997). Recent work has revealed a mammalian inhibitor of IAPs, DIABLO/Smac, that inhibits the IAPs and thus promotes caspase-9, -3 and -7 activities (Du et al. 2000; Verhagen et al. 2000). Similar regulation of the caspase-2 pathway has not been found. The death adaptor protein RAIDD activates caspase-2 (Duan and Dixit 1997) but, to date, no IAPs have been found which bind caspase-2 (Deveraux et al. 1999b). The position of caspase-2 in an activation cascade has not been clarified. It has been proposed to act as either an activator or an effector. In any event, it does appear to be independent of the caspase-9 pathway.

These studies reported reveal specific up-regulation in brains and sympathetic neurons of caspase-2 null mice of both caspase-9 and of DIABLO/Smac. As a consequence of these changes, TFD-induced death, which is normally dependent on caspase-2, switches to an alternative pathway dependent on caspase-9. These results show the tight regulation of caspase activities in neurons. When caspase-2 is removed early in development, as in the null animals, the redundancy of caspases allows the compensations which we have described. The shift from the caspase-2 to the caspase-9 pathway would assure the elimination of neurons which are superfluous. This regulated expression of caspases and IAP inhibitors is likely to be important in neurodegenerative disorders as well as in neurodevelopment.

Materials and Methods

Sympathetic Neuron Cultures

Sympathetic neuron cultures were prepared from 1-day-old wild-type and caspase-2−/− mouse pups (Bergeron et al. 1998). Cultures were grown in 24-well collagen-coated dishes for survival experiments and in 6-well collagen-coated dishes for RNA and protein extraction in RPMI 1640 medium plus 10% horse serum with mouse NGF (100 ng/ml). One day following plating, uridine and 5-fluorodeoxyuridine (10 μM each) were added to the cultures and left for three days to eliminate non-neuronal cells (less than 1% non-neuronal cells remain after 3 days). For survival experiments, on the sixth day following plating, NGF was removed by washing the cultures three times with RPMI 1640 medium plus 10% horse serum, followed by the addition of medium containing anti-mouse NGF (1:200, Sigma). Caspase inhibitors (Enzyme Systems Products) were added as indicated. Each culture was scored, as previously described (Troy et al., 1997), for numbers of living, phase-bright neurons present in the same field at various times. Three replicate cultures were assessed for each condition and data are normalized to numbers of neurons present in each culture at the time of NGF deprivation and reported as mean±SEM. For RNA and protein extraction on the sixth day following plating, RNA and protein were extracted using the Trizol reagent according to the manufacturer's protocol.

Synthesis of Antisense Oligonucleotides

Oligonucleotides bearing an SH group at their 5′ end and an NH group at their 3′ end were synthesized by Operon (California). As previously described (Troy et al., 1996a), oligonucleotides were resuspended in deionized water, an equimolar ratio of PENETRATIN1™ (Oncor) was added and the mixture was incubated at 37° C. for 1 hour. The yield of the reaction, estimated by SDS-PAGE followed by Coomassie blue staining, was routinely above 50%. As a control, a scrambled sequence of the antisense oligonucleotide (same base composition, different order) was used. Antisense sequences used were:

Acasp1 =
CCTCAGGACCTTGTCGGCCAT
ACasp3 =
GTTGTTGTCCATGGTCACTTT
Acasp6 =
TGTTTCCATCATGCTTTATTG
Acasp7N1 =
ATCGTCTGTCATCGTTCCCAC
Acasp7N2 =
CTCGAAGTCCATACGGTACAG
Acasp8 =
GTGGAAATCCATTCTTACCAA
Acasp9 =
CTGCCGGTCCGCCTCGTCCAT
Adiablo =
AGAGCCGCCATCCCGCGGCCA
AAPAF1 =
CTTTGCATCCATTGTGCCTCA
AMIAP3 =
GTTAAAAGTCATCTTCTCTGG

Western Blotting

Postnatal day 1 mouse brains were harvested in sample buffer. For antisense down-regulation studies, PC12 cells, grown as previously described, were treated with various antisense constructs for 5 hours and harvested in sample buffer. Equal amounts of protein were separated by 15% PAGE, transferred to nitrocellulose and immunostained as described (Troy et al, 2000). Anti-caspase-9 (MBL) was used at 1:1000, anti-APAF-1 (StressGen) was used at 1:1000, anti-Smac was used at 1:2000, anti-XIAP (StressGen) was used at 1:1000, anti-RAIDD (StressGen) was used at 1:500 and anti-actin (Sigma) was used at 1:200. Visualization was with ECL, using goat-anti-rabbit peroxidase at 1:1000. The relative intensities of the protein bands were quantified using Scion Image 1.55 software (NIH).

Quantitative PCR

Primers were designed to amplify a 300-400 base piece of each gene of interest. cDNA from brains of wild-type and caspase-2-null mice or cDNA from cultured sympathetic neurons were added to a reaction mix together with appropriate primers at 0.5 μM each. Reaction mix for the Roche Light Cycler was DNA Master SYBR Green 1 (Roche Molecular Biochemicals). Reaction mix for the Cepheid SMARTCYCLER™, (Fisher) was PCR READY-TO-GO BEADS™ (Amersham Pharmaceuticals) with SYBR GREEN™ (Molecular Probes). Levels of gene transcripts were analyzed using the Roche LIGHT CYCLER™ the Cepheid SMARTCYCLER™ following the manufacturers' specifications. Real time fluorescence of SYBR GREEN™ indicated that double-stranded DNA was measured. Melting curve analysis was used for each protocol to characterize and identify the specific amplicon. In each case quantification was made from the linear portion of the amplification curve. Actin was used to normalize input cDNA.

Immunocytochemistry

Sympathetic neurons were grown on.collagen-coated 8 well LabTek chamber slides. After indicated treatments, cells were fixed with 4% paraformaldehyde and immunostained as previously described (Troy et al. 1997). Cells were double labeled with anti-actin (Sigma) at 1:250 and anti-activated caspase-3 (New England Biolabs) at 1:100. Western blotting showed that the lot of the activated caspase-3 antibody used for these studies detected activated caspase-3 but not caspase-3 zymogen. Secondary antibodies were goat-anti-rabbit Alexafluor 546 and goat-anti-mouse Alexafluor 488 (Molecular Probes), both at 1:1000. Cells were examined with a Perkin-Elmer Spinning Disc confocal imaging system mounted on a Nikon inverted microscope.

Results

Caspase-2-null Neurons Employ an Alternate Caspase Path to Death after Trophic Factor Deprivation

Caspase-2 has been identified as critical for trophic factor death in sympathetic neurons and PC12 cells (Troy et al. 1997; Haviv et al. 1998). However, cultured sympathetic neurons from caspase-2-null mice die when deprived of NGF (Bergeron et al. 1998). To ascertain that TFD-induced death in the caspase-2-null neurons was caspase-dependent, both wild-type and caspase-2-null cells were treated with the pseudosubstrate caspase inhibitors BAF and DEVD-FMK. FIG. 1A shows that-the broad-spectrum caspase inhibitor BAF protects caspase-2-null neurons as well as wild-type neurons, confirming that the death process is caspase-mediated in both sets of neurons. However, DEVD-FMK, used at a concentration (10 μM) that is relatively specific for caspase-3 family members, provided protection only for caspase-2-null neurons (FIG. 1B). This suggested that although caspase activity was required for death in both cases after removal of NGF, different caspases were used in each case. The rescue of the caspase-2-null neurons from TFD by DEVD-FMK suggested that this was a member of the caspase-3 family.

Caspase-2 Null Mice Brains and Sympathetic Neurons have Increased Expression of Caspase-9 and DIABLO/Smac

The differential effects of DEVD-FMK led us to investigate whether the targeted knock-out of caspase-2 resulted in changes in expression of other caspases or other constituents of cell death pathways that would preserve vulnerability to TFD. Brains, not including cerebella, from wild-type and caspase-2-null postnatal day 1(P1) mice were harvested for RNA and protein, and the relative expression of various caspases was determined using quantitative PCR and Western blotting. These studies revealed that caspase-2 and caspase-9 transcripts are differentially expressed in the two groups of animals; caspase-2-null animals have no caspase-2 mRNA but have more than 3 times the levels of caspase-9 mRNA, relative to actin expression (FIG. 2A). No significant changes were observed for transcripts encoding caspases-1, -3, -6, -7, -8, -11, or -14. The increase in caspase-9 mRNA was confirmed by Northern blotting. Western blotting also revealed changes only in caspase-2 and caspase-9 levels. Caspase-9 protein was increased approximately three-fold in the caspase-2-null mouse brain (FIG. 2B) and, as expected, caspase-2 protein was absent. Other caspases (-1, -3, -6, -7, -8, -11) were unchanged (caspase-3 levels are shown in FIG. 2B). The increase in caspase-9 expression was confirmed in cultured sympathetic neurons as well. Sympathetic neurons from wild-type and caspase-2-null P1 animals were cultured for 5 days and harvested for total cellular RNA and protein assays. Quantitative PCR showed a more than 5-fold increase in caspase-9 expression (FIG. 2E). Western blotting confirmed the increase in caspase-9 protein in the neurons.

Next, it was investigated whether loss of caspase-2 resulted in changes in other molecules known to modulate the caspase-2 or caspase-9 pathways. These include RAIDD for.caspase-2, and Apaf-1, MIAP3 and DIABLO/Smac for caspase-9. There were no changes in expression of either message or protein for RAIDD, the death adaptor protein for caspase-2 (Duan and Dixit 1997) or Apaf-1, the mammalian ced-4 homologue that activates caspase-9 (Zou et al. 1997) (FIGS. 2C, 2D). However, in the case of the recently discovered DIABLO/Smac, an inhibitor of IAPs (inhibitor of apoptosis proteins) that is permissive for caspase-9 activation (Chai et al. 2000; Du et al. 2000; Verhagen et al. 2000), both mRNA and protein were increased by approximately two-fold in brain, as well as in sympathetic neurons (FIGS. 2C, 2D, 2E). MIAP3, a mouse homologue of XIAP (Farahani et al. 1997), an IAP that has been shown to inhibit the activity of caspases-3, -7 and -9 (Takahashi et al. 1998; Deveraux et al. 1997, 1999a), was unchanged (FIGS. 2C, 2D, 2E).

Specific Inhibition of the Caspase-9 Pathway Protects Caspase-2-null Neurons but not Wild-type Neurons from TFD

It was next assessed whether, as indicated by the above findings, TFD-induced death of caspase-2 null and wild-type sympathetic neurons employs different sets of caspases. Since none of the available pharmacologic caspase inhibitors is completely specific for an individual caspase, antisense oligonucleotides were used to decrease expression of specific caspases. This was achieved by using antennapedia peptide (PENETRATIN1™)-mediated intracellular delivery of antisense oligonucleotides, which is a technique that has been widely and successfully used for such purposes (Allinquant et al. 1995; Troy et al. 1996; Pooga et al. 1998; Nakagawa et al. 2000). By this means, 50-80% down-regulation of individual caspases was achieved in cultured neuronal cells without affecting levels of other caspases (Troy et al. 1997; Troy et al. 2000, and representative blots in FIG. 3A). Control (scrambled) oligonucleotides had no discernable effect on expression. Antisense oligonucleotides were designed to down-regulate caspases-1, -2, -3, -6, -7, -8, -9 and linked to PENETRATIN1™. Specificity of sequences was verified by BLAST search (NCBI). Efficacy of down-regulation was evaluated by Western blotting of the target caspase in PC12 cell cultures with or without exposure to the PENETRATIN1™-linked oligonucleotides (FIG. 3A for caspases-6, -7, -8, -9; Troy et al. 1997 for caspase-2; Troy et al. 2000 for caspases-1, -3). PC12 cell cultures were used because of the greater number amount of material available for for the biochemical measurements. Our previous work supports the concurrence of mechanisms in PC12 cells and sympathetic neurons (Farinelli et al. 1996; Park et al. 1998; Stefanis et al. 1998; Troy et al. 1997, 2000). All antisense oligonucleotides provided greater than 50% downregulation of the targeted caspase within 5 hours of treatment. Levels of the other non-targeted caspases were not affected (data not shown). Sympathetic neurons from wild-type and caspase-2 null mice were deprived of NGF in the presence and absence of each of these antisense oligonucleotides and survival assessed daily for three days (FIGS. 3B, 3C). As previously shown, PENETRATIN1™-linked antisense oligonucleotide to caspase-2 (V-ACasp2, previously called V-ANedd) protected wild-type neurons from NGF withdrawal. As anticipated, there was no protection of caspase-2-null neurons by this construct. In contrast, caspase-2-null neurons were protected by V-ACasp3, V-ACasp7 and V-ACasp9. These antisense constructs, however, provided no protection for wild-type neurons. No protection was afforded for either wild-type or caspase-2-null neurons by control (scrambled) oligonucleotides or by downregulation of caspases-1, -6 or -8.

Downregulation of DIABLO/Smac or Apaf-1 Selectively Protects Caspase-2-null Neurons from TFD

It was next tested whether down-regulation of additional components of the caspase-9 pathway would bring about differential protection. Antisense-mediated down-regulation of either DIABLO/Smac (FIGS. 4A, 4B) or Apaf-1 (FIGS. 4D, 4E) provided complete protection against NGF withdrawal for caspase-2-null neurons but had no effect on survival of wild-type neurons. FIG. 4C shows the efficacy of down-regulation by these constructs. The photomicrographs in FIGS. 5C-5F show that inhibition of Apaf-1 (FIG. 5C), DIABLO/Smac (Figure 5D), caspase-9 (FIG. 5E) or caspase-3 (FIG. 5F) expression in caspase-2-null neurons protected not only cell bodies, but also neurites.

Downregulation of DIABLO/Smac, Apaf-1, Caspase-9 or Caspase-3 Suppresses Elevation of Activated Caspase-3 in NGF-deprived Neurons from Caspase-2 null Mice

The preceding findings point to the activation of.the caspase-9 pathway in caspase-2-null neurons, with consequent activation of caspases-3 and -7. Using an antibody that specifically recognizes activated caspase-3 (see Methods), we assessed the cellular localization of this enzyme in caspase-2-null neurons after various treatments. The confocal micrographs in FIGS. 6A-6F depict cultures of sympathetic neurons from caspase-2-null mice double-labeled for actin (green, but color not shown) and activated caspase-3 (red, but color not shown). Control cells show only minimal staining for activated caspase-3 in either cell bodies and neurites (FIG. 6A). After 5 hours of TFD, there is substantial activation of caspase-3. In the two cells shown in FIG. 6B, it is clear that, as activation of caspase-3 increases, actin immunostaining decreases, likely due to actin degradation during the death process. The induction of activated caspase-3 seen in caspase-2-null neurons after TFD is blocked by downregulation of either Diablo or APAF-1 with the appropriate antisense oligonucleotide. Downregulation of caspase-9 or caspase-3 (FIGS. 6E, 6F) substantially decreased the amount of activated caspase-3 detectable by immunostaining, but did not completely block it.

The Caspase-9 Pathway is Suppressed in Wild-type Neurons by IAPs

Although NGF-deprivation induces DEVDase activity in wild-type sympathetic neurons and PC12 cells, this is neither necessary nor sufficient to induce death (Troy et al. 1997; Stefanis et al. 1998). In addition, endogenous suppressors of caspases are likely to play an important role in the regulation of caspase activity and death. The IAP family of caspase inhibitors has been shown to block caspases-3, -7 and -9 activities (Deveraux et al. 1997, 1999b). To reduce IAP activity in cultured sympathetic neurons, a PENETRATIN1™-linked antisense oligonucleotide (V-AMIAP3) to MIAP3 was designed. MIAP3 was chosen because it is the mouse homologue of XIAP, the IAP that has been most closely linked with the caspase-9 pathway. V-AMIAP3 promotes 70% down-regulation of MIAP3 within 5 hours (FIG. 7A). To determine if in vivo activation of the caspase-9 pathway might be suppressed by IAPs in wild-type neurons, we withdrew NGF in the presence of V-ACasp2 and V-AMIAP3. Simultaneous treatment with multiple PENETRATIN1™-linked antisense oligonucleotides does not alter the effects of the individual oligonucleotides (Troy et al. 1996). As shown in FIG. 7B, the protection conferred by caspase-2 down-regulation (by V-ACasp2) was reversed by down-regulation of MIAP3 (by co-treatment with V-AMIAP3). This suggests that reduction of MIAP3 levels permits death by an otherwise suppressed caspase-9-dependent pathway. Consistent with this, the death induced by V-ACasp2 plus V-AMIAP3 was prevented by down-regulation of either Apaf-1 or caspase-9 (FIG. 7B). This suggestion was further supported when we examined immunostaining of activated caspase-3 in these neurons. Withdrawal of NGF in the presence of V-ACasp2plus V-AMIAP3 induced a strong signal in both cell bodies and neurites (FIGS. 7C, 7D). This activated caspase-3 immunostaining was suppressed by the downregulation of caspase-9 (FIG. 7E).

Discussion

In an attempt to reconcile the apparently conflicting observations that NGF withdrawal induces apoptosis in caspase-2-null sympathetic neurons (Bergeron et al. 1998), but is unable to cause death in neurons in which caspase-2 has been down regulated (Troy et al. 1997), we examined the ability of caspase inhibitors to rescue each of these cell types from death. Both cell types were rescued by the broad spectrum inhibitor, BAF, but only the caspase-2-null neurons were rescued by DEVD-FMK, which is relatively selective for caspase-3-like activities when used at 10 μM. This differential sensitivity to the caspase inhibitors led us to question whether there were differences in the expression of caspases or the regulators of caspase activity in these two cell types. We found that caspase-9 mRNA and protein are selectively increased by approximately 3-fold in the newborn caspase-2-null mouse brain and more than 5-fold in cultured sympathetic neurons. Expression of the pro-apoptotic death regulator DIABLO/Smac was also elevated.

The increases in the expression of these two pro-apoptotic molecules suggests that they might compensate for the loss of caspase-2 and enable neurons from caspase-2-null mice to die by an alternative pathway mediated by caspase-9 and its downstream targets such as caspases-3 and -7. This was confirmed in the series of experiments showing that down-regulation of caspase-2 suppresses TFD in wild-type neurons, but not in caspase-2-null neurons. In contrast, down-regulation of caspases-3, -7, and -9 rescues caspase-2-null neurons, but not wild-type neurons, from NGF deprivation. Furthermore, interference with caspase-9 activation by down-regulation of Apaf-1 provided protection for caspase-2-null neurons, but not for wild-type neurons.

The compensatory switch to the caspase-9 pathway that we observed in caspase-2-null mice appears to involve more than simply elevation of caspase-9 levels. DIABLO/Smac is a recently identified protein that enables activation of caspase-9 (and most likely, caspases-3 and -7) by binding to members of the IAP (inhibitor of apoptosis protein) family (Chai et al. 2000; Du et al. 2000; Verhagen et al. 2000). We observed that DIABLO/Smac levels are doubled in caspase-2-null brains and sympathetic neurons and that down-regulation of DIABLO/Smac protects caspase-2-null, but not wild-type, sympathetic neurons from NGF deprivation. Thus, the availability of the caspase-9 pathway for induction of death in NGF-deprived neurons may be at least in part dependent on its regulation by the competing activities of IAPs and DIABLO/Smac. In caspase-2-null neurons, the elevated levels of DIABLO/Smac might help swing the balance to favor enhanced activation of the caspase-9 pathway.

The apparent involvement of DIABLO/Smac in promoting death of caspase-2-null neurons raised the issue of whether IAPs may play a role in the repression of the caspase-9 pathway in wild-type neurons. The IAPs effectively suppress activity of caspases-3, -7 and -9 (Deveraux et al. 1997; Deveraux et al. 1998; Deveraux et al. 1999b). We chose to investigate the role of MIAP3, the mouse homologue of XIAP, because it is expressed in sympathetic neurons and can inhibit all three of the above caspases (Farahani et al. 1997). We found that although downregulation of caspase-2 protects wild-type neurons from NGF deprivation, the simultaneous down-regulation of MIAP3 results in death. This death appeared to involve the caspase-9 pathway because it was inhibited in turn by the additional down-regulation of either caspase-9 or Apaf-1. The simplest interpretation of these observations is that when NGF is removed from wild-type neurons in our system, caspase-2 is activated and mediates death, and that the caspase-9 pathway is held in check by MIAP3. When MIAP3 is down-regulated in such neurons, the caspase-9 pathway is no longer suppressed and, when caspase-2 is also down-regulated, the caspase-9 pathway mediates death. A schematic depiction of the alternative death pathways is shown in FIGS. 8A and 8B.

Taken together, our findings support the idea that wild-type sympathetic neurons possess two alternative caspase pathways that have the potential to mediate TFD-induced death. Under the conditions of our experiments, the caspase-2 pathway is predominant and the caspase-9 pathway is held in check by IAPs. One result of this arrangement is that various circumstances may switch utilization of the two pathways. For instance, as we observed, knockout of caspase-2 results in compensatory enablement of the caspase-9 pathway due, at least in part, to up-regulation of caspase-9 and of DIABLO/Smac. Another variable is developmental stage. Although the existing literature is incomplete, it appears that caspase 9 is highly expressed in the developing mouse brain at embryonic day 7 and declines after that (Kuida et al. 1998).

In contrast, mouse caspase-2 (originally identified by virtue of its down-regulation in brain during development (Kumar et al. 1994)) is barely expressed at embryonic day 8, and has peak expression in the brain at embryonic day 12. However, rodent sympathetic neurons show high expression of caspase-2 in P1 animals and a subsequent decrease so that expression is minimal by day P11 (Savitz and Kessler 2000). Developmental expression patterns for other elements of either the caspase-2 or caspase-9 pathways have yet to be established in sympathetic neurons, but such time-dependent changes represent potentially important variables in choice of caspase death mechanisms. Similarly, it is likely that additional factors can influence the expression of specific caspases and caspase regulatory molecules, and thereby switch cells from one death pathway to another.

In light of the aforementioned findings, it is not surprising that circumstances may occur in which TFD-induced death of sympathetic neurons is dependent on the caspase-9 pathway. It was recently reported that sympathetic neurons from caspase-9 null embryos undergo delayed TFD-induced death (Deshmukh et al. 2000) and on this basis, it was suggested that caspase-9 plays a critical role in death caused by NGF deprivation. These studies employed E17 embryos, a developmental stage at which the expression of caspase-9 may normally be higher and caspase- 2 lower than in the postnatal (P1) neurons used in our studies. It is also possible that the knock-out of caspase-9 also leads to depression of the caspase-2 pathway and delay of death.

The failure of caspase 3 activation to cause death in the wild-type neurons in which caspase-2 has been -down-regulated suggests that the level of activation falls below a critical level for inducing apoptosis. This possibility is supported by the observation that further increasing the activity of the caspase-9 pathway by down-regulation of MIAP3 leads to death and by the increased concentrations of both caspase-9 and DIABLO/Smac in the caspase-2-null mouse brains and neurons. It is possible that “subapoptotic” activation of the caspases in the caspase-9 pathway serves one or more important functions, such as mediating cytoskeletal breakdown.

In contrast to the caspase 9 pathway, relatively little is known about the mechanisms by which caspase-2 is activated and how such activation leads to death. Our past work indicates that caspase-2 is not downstream of caspase-3-like activity in NGF-deprived sympathetic neurons and visa versa (Stefanis et al. 1998). Caspase-2 possesses a long CARD-containing pro-domain that appears important for activation via specific association with CARD-containing adapter proteins such as RAIDD (Duan and Dixit 1997). However, little is known about how NGF deprivation might trigger interaction between caspase-2 and RAIDD and/or other activators. Several types of evidence indicate that cytochrome C release from mitochondria is required for TFD-induced death of sympathetic neurons (Deshmukh and Johnson 1998; Neame et al. 1998). Given the importance of caspase-2 in TFD-induced death, this raises the yet untested possibility that activation of caspase-2 lies downstream of mitochondrial perturbation. In this regard, it may be relevant that procaspase-2 has been reported to be present within mitochondria and to be released during the apoptotic process (Susin et al. 1999). Also, in contrast to the caspase-9 pathway, there are no currently known negative regulators of caspase-2 activity. Thus it is possible that, unlike caspases -3, -7 and -9 which are subject to inhibition by IAPs, caspase-2, once it is activated, inevitably leads to death.

The aforementioned findings raise a note of caution regarding interpretation of data from knock-out animals and emphasize the flexibility and redundancy of apoptotic pathways. Two compensatory changes were discovered in expression of apoptosis-related proteins that occur in response to loss of caspase-2 and these appear to contribute to enablement of an alternative apoptotic caspase pathway in sympathetic neurons. It is conceivable that similar or additional changes also occurred in other tissues and that these may affect other aspects of the phenotype of caspase-2-null mice. It has been recently reported that compensatory changes in caspase activation occur in caspase-9 and caspase-3-null animals (Zheng et al. 2000). Although the latter study did not identify specific molecular changes that underlie the compensatory activation of alternative caspases, it does underscore the point raised here that cells possess multiple apoptotic caspase pathways and the means to switch from one to another.

In summary, the data presented here show that TFD-induced death of sympathetic neurons has the potential to proceed by either of two distinct pathways and that the decision of which pathway is used in a given situation can be regulated by alterations in the relative levels of the components of each of the pathways. It is of particular interest that such regulation included both caspases and an IAP inhibitor. While we have manipulated these levels by genetic and antisense approaches, they are likely regulated to similar effect both during development and in neurodegenerative disorders.

References

1. Allinquant, B., et al., (1995). “Downregulation of amyloid precursor protein inhibits neurite outgrowth in vitro.” J Cell Biol 128(5):919-27.

2. Bergeron, L., et al., (1998). “Defects in regulation of apoptosis in caspase-2-deficient mice.” Genes Dev 12(9): 1304-14.

3. Chai, J., et al., (2000). “Structural and biochemical basis of apoptotic activation by Smac/DIABLO.” Nature 406(6798):855-62.

4. Deshmukh, M., et al., (1998). “Evidence of a novel event during neuronal death: development of competence-to-die in response to cytoplasmic cytochrome c.” Neuron 21(4): 695-705.

5. Deshmukh, M., et al., (2000). “Caspase inhibition extends the commitment to neuronal death beyond cytochrome c release to the point of mitochondrial depolarization.” J Cell Biol 150(1):131-43.

6. Deveraux, Q. L., et al., .(1999a). “Cleavage of human inhibitor of apoptosis protein XIAP results in fragments with distinct specificities for caspases.” Embo J 18(19): 5242-51.

7. Deveraux, Q. L., et al., (1998). “IAPs block apoptotic events induced by caspase-8 and cytochrome c by direct inhibition of distinct caspases.” Embo J 17(8):2215-23.

8. Deveraux, Q. L., et al., (1999b). “Endogenous inhibitors of caspases.” J Clin Immunol 19(6):388-98.

9. Deveraux, Q. L., et al., (1997). “X-linked IAP is a direct inhibitor of cell-death proteases.” Nature 388(6639):300-4.

10. Du, C., et al., (2000). “Smac, a mitochondrial protein that promotes cytochrome c-dependent caspase activation by eliminating IAP inhibition.” Cell 102(1):33-42.

11. Duan, H. and V. M. Dixit (1997). “RAIDD is a new ‘death’ adaptor molecule.” Nature 385(6611):86-9.

12. Farahani, R., et al., (1997). “Genomic organization and primary characterization of miap-3: the murine homologue of human X-linked IAP.” Genomics 42(3):514-8.

13. Farinelli, S. E., et al., (1996). “Nitric oxide delays the death of trophic factor-deprived PC12 cells and sympathetic neurons by a cGMP-mediated mechanism.” J Neurosci 16(7):2325-34.

14. Hakem, R., et al., (1998). “Differential requirement for caspase 9 in apoptotic pathways in vivo.” Cell 94(3): 339-52.

15. Haviv, R., et al., (1998). “Need for caspase-2 in apoptosis of growth-factor-deprived PC12 cells.” J Neurosci Res 52(5):491-7.

16. Hengartner, M. 0. (2000). “The biochemistry of apoptosis.” Nature 407(6805):770-6.

17. Kuida, K., et al., (1998). “Reduced apoptosis and cytochrome c-mediated caspase activation in mice lacking caspase 9.” Cell 94(3):325-37.

18. Kuida, K., et al., (1996). “Decreased apoptosis in the brain and premature lethality in CPP32-deficient mice.” Nature 384(6607):368-372.

19. Kumar, S., et al., (1994). “Induction of apoptosis by the mouse Nedd2 gene, which encodes a protein similar to the product of the Caenorhabditis elegans cell death gene ced-3 and the mammalian IL-1 beta-converting enzyme.” Genes Dev 8(14):1613-26.

20. Nakagawa, T., et al., (2000). “Caspase-12 mediates endoplasmic-reticulum-specific apoptosis and cytotoxicity by amyloid-beta.” Nature 403(6765):98-103.

21. Neame, S. J., et al., (1998). “Blocking cytochrome c activity within intact neurons inhibits apoptosis.” J Cell Biol 142(6):1583-93.

22. Park, D. S., et al., (1998). “Multiple pathways of neuronal death induced by DNA-damaging agents, NGF deprivation, and oxidative stress.” J Neurosci 18(3): 830-40.

23. Pettmann, B. and C. E. Henderson (1998). “Neuronal cell death.” Neuron 20(4):633-47.

24. Pooga, M., et al., (1998). “Cell penetrating PNA constructs regulate galanin receptor levels and modify pain transmission in vivo.” Nat Biotechnol 16(9):857-61.

25. Salvesen, G. S. (1999). “Programmed cell death and the caspases.” Apmis 107(1):73-9.

26. Savitz, S. I. and J. A. Kessler (2000). “Leukemia inhibitory factor requires concurrent p75LNTR signaling to induce apoptosis of cultured sympathetic neurons.” J Neurosci 20(11):4198-205.

27. Stefanis, L., et al., (1998). “Caspase-2 (Nedd-2) processing and death of trophic factor-deprived PC12 cells and sympathetic neurons occur independently of caspase-3 (cPP32)-like activity.” J Neurosci 18(22): 9204-15.

28. Susin, S. A., et al., (1999). “Mitochondrial release of caspase-2 and -9 during the apoptotic process.” J Exp Med 189(2):381-94.

29. Takahashi, R., et al., (1998). “A single BIR domain of XIAP sufficient for inhibiting caspases.” J Biol Chem 273(14):7787-90.

30. Thornberry, N. A. and Y. Lazebnik (1998). “Caspases: enemies within.” Science 281:1312-6.

31. Troy, C. M., et al., (1996). “Downregulation of Cu/Zn superoxide dismutase leads to cell death via the nitric oxide-peroxynitrite pathway.” J Neurosci 16(1):253-61.

32. Troy, C. M., et al., (2000). “Caspase-2 mediates neuronal cell death induced by beta-amyloid J Neurosci 20(4):1386-92.

33. Troy, C. M., et al., (1997). “Nedd2 is required for apoptosis after trophic factor withdrawal, but not superoxide dismutase (SOD1) downregulation, in sympathetic neurons and PC12 cells.” J Neurosci 17(6): 1911-8.

34. Verhagen, A. M., et al., (2000). “Identification of DIABLO, a mammalian protein that promotes apoptosis by binding to and antagonizing IAP proteins.” Cell 102(1): 43-53.

35. Yoshida, H., et al., (1998). “Apafl is required for mitochondrial pathways of apoptosis and brain development.” Cell 94(6):739-50.

36. Zheng, T. S., et al., (2000). “Deficiency in caspase-9 or caspase-3 induces compensatory caspase activation.” Nat Med 6(11):1241-7.

37. Zou, H., et al., (1997). “Apaf-1, a human protein homologous to C. elegans CED-4, participates in cytochrome c-dependent activation of caspase-3.” Cell 90(3):405-13.

What is claimed is:

1. A nucleic acid which specifically hybridizes to a nucleic acid encoding an inhibitor-of-apoptosis protein.

2. The nucleic acid of claim 1, wherein the nucleic acid is complementary to the nucleic acid encoding the inhibitor-of-apoptosis protein.

3. The nucleic acid of claim 1, wherein the nucleic acid has a length of from about 15 nucleotides to about 25 nucleotides.

4. The nucleic acid of claim 1, wherein the inhibitor-of-apoptosis protein is selected from the group consisting of MIAP1, MIAP2, MIAP3, CIAP1, CIAP2, and XIAP.

5. The nucleic acid of claim 1, wherein the nucleic acid specifically hybridizes to the portion of the nucleic acid encoding MIAP3 beginning with the adenosine at position 769 and ending with the guanosine at position 791.

6. A composition comprising the nucleic acid of claim 1 and a carrier.

7. The composition of claim 6, wherein the composition comprises nucleic acids which specifically hybridize to nucleic acids encoding a plurality of inhibitor-of-apoptosis proteins.

8. The composition of claim 7, wherein the inhibitor-of-apoptosis proteins comprise CIAP1, CIAP2 and XIAP.

9. The composition of claim 7, wherein the inhibitor-of-apoptosis proteins comprise CIAP1 and XIAP.

10. The composition of claim 7, wherein the inhibitor-of-apoptosis proteins comprise CIAP2 and XIAP.

11. The composition of claim 7, wherein the inhibitor-of-apoptosis proteins comprise CIAP1 and CIAP2.

12. The composition of claim 6, wherein the carrier comprises a diluent, an adjuvant, a virus, a liposome, a microencapsule, a neuronal cell receptor ligand, a neuronal-specific virus, a polymer-encapsulated cell or a retroviral vector.

13. The composition of claim 6, wherein the carrier is an aerosol, an intravenous carrier, an oral carrier or a topical carrier.

14. A method for inducing a cell's death which comprises contacting the cell with the nucleic acid of claim 1 under conditions permitting the nucleic acid to enter the cell.

15. The method of claim 14, further comprising contacting the cell with nucleic acids which specifically hybridize to nucleic acids encoding a plurality of inhibitor-of-apoptosis proteins.

16. The method of claim 15, wherein the inhibitor of apoptosis proteins comprise CIAP1, CIAP2 and XIAP.

17. The method of claim 15, wherein the inhibitor of apoptosis proteins comprise CIAP1 and XIAP.

18. The method of claim 15, wherein the inhibitor of apoptosis proteins comprise CIAP2 and XIAP.

19. The method of claim 15, wherein the inhibitor of apoptosis proteins comprise CIAP1 and CIAP2.

20. The method of claim 14, wherein the conditions permitting the nucleic acid to enter the cell comprise the use of a vector, a liposome, a mechanical means or an electrical means.

21. The method of claim 20, wherein the vector is selected from the group consisting of an adenovirus vector, an adeno-associated virus vector, an Epstein-Barr virus vector, a Herpes virus vector, an attenuated HIV vector, a retroviral vector and a vaccinia virus vector.

22. The method of claim 20, wherein the liposome is an antibody-coated liposome.

23. A method for treating a subject afflicted with cancer which comprises administering to the subject a therapeutically effective amount of the nucleic acid of claim 1.

24. The method of claim 23, wherein the cancer is selected from the group consisting of acute lymphocytic leukemia, acute myelogenous leukemia, lung cancer, breast cancer, ovarian cancer, prostate cancer, lymphoma, Hodgkin's disease, malignant melanoma, neuroblastoma, renal cell carcinoma and squamous cell carcinoma.

25. The method of claim 23, wherein the cancer is a tumor.

26. The method of claim 23, wherein the subject is a mammal.

27. The method of claim 26, wherein the subject is a human.

28. An nucleic acid that specifically hybridizes to a nucleic acid which encodes a protein, other than caspase-2, that induces cell death.

29. The nucleic acid of claim 28, wherein the nucleic acid is complementary to the nucleic acid encoding the protein that induces cell death.

30. The nucleic acid of claim 28, wherein the nucleic acid has a length of from about 15 nucleotides to about 25 nucleotides.

31. The nucleic acid of claim 28, wherein the protein is selected from the group consisting of APAF1, RAIDD, and Diablo/SMAC.

32. The nucleic acid of claim 28 which specifically hybridizes to a nucleic acid encoding the protein APAF1.

33. The nucleic acid of claim 28, wherein the nucleic acid specifically hybridizes to the portion of the nucleic acid encoding APAF-1 beginning with the cytosine at position 576 and ending with the adenosine at position 596.

34. The nucleic acid of claim 28 which specifically hybridizes to a nucleic acid encoding the protein RAIDD.

35. The nucleic acid of claim 28, wherein the nucleic acid specifically hybridizes to the portion of the nucleic acid encoding RAIDD beginning with the guanosine at position 110 and ending with the adenosine at position 130.

36. The nucleic acid of claim 28 which specifically hybridizes to a nucleic acid encoding the protein Diablo/SMAC.

37. The nucleic acid of claim 28, wherein the nucleic acid specifically hybridizes to the portion of the nucleic acid encoding Diablo/SMAC beginning with the thymidine at position 1 and ending with the thymidine at position 21.

38. A composition comprising the nucleic acid of claim 28 and a carrier.

39. The composition of claim 38, wherein the composition comprises nucleic acids which specifically hybridize to nucleic acids encoding a plurality of proteins that induce cell death.

40. The composition of claim 39, wherein the proteins comprise APAF-1 and Diablo/SMAC.

41. The composition of claim 39, wherein the proteins comprise APAF-1, Diablo/SMAC and caspase-9.

42. The composition of claim 39, wherein the proteins comprise APAF-1, Diablo/SMAC and caspase-7.

43. The composition of claim 39, wherein the proteins comprise caspase-2 and RAIDD.

44. The composition of claim 39, wherein the proteins comprise caspase-8 and RAIDD.

45. The composition of claim 39, wherein the proteins comprise caspase-8, RAIDD and caspase-3.

46. The composition of claim 39, wherein the proteins comprise caspase-2 and caspase-9.

47. The composition of claim 38, wherein the carrier comprises a diluent, an adjuvant, a virus, a liposome, a microencapsule, a neuronal cell receptor ligand, a neuronal-specific virus, a polymer-encapsulated cell or a retroviral vector.

48. The composition of claim 38, wherein the carrier is an aerosol, an intravenous carrier, an oral carrier or a topical carrier.

49. A method for inhibiting a cell's death which comprises contacting the cell with the nucleic acid of claim 28 under conditions permitting the nucleic acid to enter the cell.

50. A method for inhibiting a neuronal cell's death which comprises contacting the cell with the nucleic acid of claim 28 under conditions permitting the nucleic acid to enter the cell.

51. The method of claim 49 or 50, further comprising contacting the cell with nucleic acids which specifically hybridize to nucleic acids encoding a plurality of proteins that induce cell death.

52. The method of claim 51, wherein the proteins that induce cell death comprise APAF-1 and Diablo/SMAC.

53. The method of claim 51, wherein the proteins that induce cell death comprise APAF-1, Diablo/SMAC and caspase-9.

54. The method of claim 51, wherein the proteins that induce cell death comprise APAF-1, Diablo/SMAC and caspase-7.

55. The method of claim 51, wherein the proteins that induce cell death comprise caspase-2 and RAIDD.

56. The method of claim 51, wherein the proteins that induce cell death comprise caspase-8 and RAIDD.

57. The method of claim 51, wherein the proteins that induce cell death comprise caspase-8, RAIDD and caspase-3.

58. The method of claim 51, wherein the proteins that induce cell death comprise caspase-2 and caspase-9.

59. The method of claim 49 or 50, wherein the conditions permitting the nucleic acid to enter the cell comprise the use of a vector, a liposome, a mechanical means or an electrical means.

60. The method of claim 59, wherein the vector is selected from the group consisting of an adenovirus vector, an adeno-associated virus vector, an Epstein-Barr virus vector, a Herpes virus vector, an attenuated HIV vector, a retroviral vector and a vaccinia virus vector.

61. The method of claim 59, wherein the liposome is an antibody-coated liposome.

62. A method for treating a neurodegenerative disorder in a subject which comprises administering to the subject a therapeutically effective amount of the nucleic acid of claim 28.

63. The method of claim 62, wherein the neurodegenerative disorder is a brain disorder or a central nervous system disorder.

64. A method for treating a heart disorder in a subject which comprises administering to the subject a therapeutically effective amount of the nucleic acid of claim 28.

65. The method of claim 64, wherein the heart disorder is cardiomyopathy.

66. The method of claim 49, 50, 62 or 64, wherein the subject is a mammal.

67. The method of claim 66, wherein the subject is a human.


SEQUENCE LISTING (22 sequences)

SEQ ID NO: 1 - protein, 600 aa, human
MVQDSAFLAKLMKSADTFELKYDFSCELYRLSTYSAFPRGVPVSERSLARAGFYYTGANDKVKCFCCGLMLDNWKQGDSPMEKHRKLYPSCNFVQTLNPANSLEASPRPSLPSTAMSTMPLSFASSENTGYFSGSYSSFPSDPVNFRANQDCPALSTSPYHFAMNTEKARLLTYETWPLSFLSPAKLAKAGFYYIGPGDRVACFACDGKLSNWERKDDAMSEHQRHFPSCPFLKDLGQSASRYTVSNLSMQTHAARIRTFSNWPSSALVHSQELASAGFYYTGHSDDVKCFCCDGGLRCWESGDDPWVEHAKWFPRCEYLLRIKGQEFVSQVQAGYPHLLEQLLSTSDSPEDENADAAIVHFGPGESSEDVVMMSTPVVKAALEMGFSRSLVRQTVQRQILATGENYRTVSDLVIGLLDAEDEMREEQMEQAAEEEESDDLALIRKNKMVLFQHLTCVTPMLYCLLSARAITEQECNAVKQKPHTLQASTLIDTVLAKGNTAATSFRNSLREIDPALYRDIFVQQDIRSLPTDDIAALPMEEQLRKLQEERMCKVCMDREVSIVFIPCGHLVVCKDCAPSLRKCPICRGTIKGTVRTFLS

SEQ ID NO: 2 - DNA, 2673 bp, human
tgggagttccccggagccctggaggaaagcaccgcaggtctgagcagccctgagccgggcagggtgggggcagtggctaaggcctagctggggacgatttaaaggtatcgcgccacccagccacaccccacaggccaggcgagggtgccacccccggagatcagaggtcattgctggcgttcagagcctaggaagtgggctgcggtatcagcctagcagtaaaaccgaccagaagccatgcacaaaactacatccccagagaaagacttgtcccttcccctccctgtcatctcaccatgaacatggttcaagacagcgcctttctagccaagctgatgaagagtgctgacacctttgagttgaagtatgacttttcctgtgagctgtaccgattgtccacgtattcagcttttcccaggggagttcctgtgtcagaaaggagtctggctcgtgctggcttttactacactggtgccaatgacaaggtcaagtgcttctgctgtggcctgatgctagacaactggaaacaaggggacagtcccatggagaagcacagaaagttgtaccccagctgcaactttgtacagactttgaatccagccaacagtctggaagctagtcctcggccttctcttccttccacggcgatgagcaccatgcctttgagctttgcaagttctgagaatactggctatttcagtggctcttactcgagctttccctcagaccctgtgaacttccgagcaaatcaagattgtcctgctttgagcacaagtccctaccactttgcaatgaacacagagaaggccagattactcacctatgaaacatggccattgtcttttctgtcaccagcaaagctggccaaagcaggcttctactacataggacctggagatagagtggcctgctttgcgtgcgatgggaaactgagcaactgggaacgtaaggatgatgctatgtcagagcaccagaggcatttccccagctgtccgttcttaaaagacttgggtcagtctgcttcgagatacactgtctctaacctgagcatgcagacacacgcagcccgtattagaacattctctaactggccttctagtgcactagttcattcccaggaacttgcaagtgcgggcttttattatacaggacacagtgatgatgtcaagtgtttttgctgtgatggtgggctgaggtgctgggaatctggagatgacccctgggtggaacatgccaagtggtttccaaggtgtgagtacttgctcagaatcaaaggccaagaatttgtcagccaagttcaagctggctatcctcatctacttgagcagctattatctacgtcagactccccagaagatgagaatgcagacgcagcaatcgtgcattttggccctggagaaagttcggaagatgtcgtcatgatgagcacgcctgtggttaaagcagccttggaaatgggcttcagtaggagcctggtgagacagacggttcagcggcagatcctggccactggtgagaactacaggaccgtcagtgacctcgttataggcttactcgatgcagaagacgagatgagagaggagcagatggagcaggcggccgaggaggaggagtcagatgatctagcactaatccggaagaacaaaatggtgcttttccaacatttgacgtgtgtgacaccaatgctgtattgcctcctaagtgcaagggccatcactgaacaggagtgcaatgctgtgaaacagaaaccacacaccttacaagcaagcacactgattgatactgtgttagcaaaaggaaacactgcagcaacctcattcagaaactcccttcgggaaattgaccctgcgttatacagagatatatttgtgcaacaggacattaggagtcttcccacagatgacattgcagctctaccaatggaagaacagttgcggaaactccaggaggaaagaatgtgtaaagtgtgtatggaccgagaggtatccatcgtgttcattccctgtggccatctggtcgtgtgcaaagactgcgctccctctctgaggaagtgtcccatctgtagagggaccatcaagggcacagtgcgcacatttctctcctgaacaagactaatggtccatggctgcaacttcagccaggaggaagttcactgtcactcccagctccattcggaacttgaggccagcctggatagcacgagacaccgccaaacacacaaatataaacatgaaaaacttttgtctgaagtcaagaatgaatgaattacttatataataattttaattggtttccttaaaagtgctatttgttcccaactcagaaaattgttttctgtaaacatatttacatactacctgcatctaaagtattcatatattcatatattcagatgtcatgagagagggttttgttcttgttcctgaaaagcagggattgcctgcactcctgaaattctcagaaagatttacaatgttggcatttatggttcagaaactagaatcttctcccgttgctttaagaaccgggagcacagatgtccatgtgttttatgtatagaaattcctgttatttattggatgacattttagggatatgaaatttttataaagaatttgtgagaaaaagttaataaagcaacataattacctctttttttttaaagaaaaaaaa

SEQ ID NO: 3 - protein, 612 aa, human
MDKTVSQRLGQGTLHQKLKRIMEKSTILSNWTKESEEKMKFDFSCELYRMSTYSAFPRGVPVSERSLARAGFYYTGVNDKVKCFCCGLMLDNWKQGDSPVEKHRQFYPSCSFVQTLLSASLQSPSKNMSPVKSRFAHSSPLERGGIHSNLCSSPLNSRAVEDFSSRMDPCSYAMSTEEARFLTYSMWPLSFLSPAELARAGFYYIGPGDRVACFACGGKLSNWEPKDDAMSEHRRHFPHCPFLENTSETQRFSISNLSMQTHSARLRTFLYWPPSVPVQPEQLASAGFYYVDRNDDVKCFCCDGGLRCWEPGDDPWIEHAKWFPRCEFLIRMKGQEFVDEIQARYPHLLEQLLSTSDTPGEENADPTETVVHFGPGESSKDVVMMSTPVVKAALEMGFSRSLVRQTVQRQILATGENYRTVNDIVSVLLNAEDERREEEKERQTEEMASGDLSLIRKNRMALFQQLTHVLPILDNLLEASVITKQEHDIIRQKTQIPLQARELIDTVLVKGNAAANIFKNSLKEIDSTLYENLFVEKNMKYIPTEDVSGLSLEEQLRRLQEERTCKVCMDREVSIVFIPCGHLVVCQECAPSLRKCPICRGTIKGTVRTFLS

SEQ ID NO: 4 - DNA, 3151 bp, human
agttatataaaatacgaagttttcaaaaagaaggctagtgcaacagaaaagctttgctaaaacagattcttagttatttgaggtaacaaaagaaagccatgtcttgaattgattcgttcttaattataacagacttatagtggaaagggccttaaacacaggcggactttataaaatgcagtcttaggtttatgtgcaaaatactgtctgttgaccagatgtattcacatgatatatacagagtcaaggtggtgatatagaagatttaacagtgagggagttaacagtctgtgctttaagcgcagttcctttacagtgaatactgtagtcttaatagacctgagctgactgctgcagttgatgtaacccactttagagaatactgtatgacatcttctctaaggaaaaccagctgcagacttcactcagttcctttcatttcataggaaaaggagtagttcagatgtcatgtttaagtccttataagggaaaagagcctgaatatatgccctagtacctaggcttcataactagtaataagaagttagttatgggtaaatagatctcaggttacccagaagagttcatgtgacccccaaagagtcctaactagtgtcttggcaagtgagacagatttgtcctgtgagggtgtcaattcaccagtccaagcagaagacaatgaatctatccagtcaggtgtctgtggtggagatctagtgtccaagtggtgagaaacttcatctggaagtttaagcggtcagaaatactattactactcatggacaaaactgtctcccagagactcggccaaggtaccttacaccaaaaacttaaacgtataatggagaagagcacaatcttgtcaaattggacaaaggagagcgaagaaaaaatgaagtttgacttttcgtgtgaactctaccgaatgtctacatattcagcttttcccaggggagttcctgtctcagagaggagtctggctcgtgctggcttttattatacaggtgtgaatgacaaagtcaagtgcttctgctgtggcctgatgttggataactggaaacaaggggacagtcctgttgaaaagcacagacagttctatcccagctgcagctttgtacagactctgctttcagccagtctgcagtctccatctaagaatatgtctcctgtgaaaagtagatttgcacattcgtcacctctggaacgaggtggcattcactccaacctgtgctctagccctcttaattctagagcagtggaagacttctcatcaaggatggatccctgcagctatgccatgagtacagaagaggccagatttcttacttacagtatgtggcctttaagttttctgtcaccagcagagctggccagagctggcttctattacatagggcctggagacagggtggcctgttttgcctgtggtgggaaactgagcaactgggaaccaaaggatgatgctatgtcagagcaccgcagacattttccccactgtccatttctggaaaatacttcagaaacacagaggtttagtatatcaaatctaagtatgcagacacactctgctcgattgaggacatttctgtactggccacctagtgttcctgttcagcccgagcagcttgcaagtgctggattctattacgtggatcgcaatgatgatgtcaagtgcttttgttgtgatggtggcttgagatgttgggaacctggagatgacccctggatagaacacgccaaatggtttccaaggtgtgagttcttgatacggatgaagggtcaggagtttgttgatgagattcaagctagatatcctcatcttcttgagcagctgttgtccacttcagacaccccaggagaagaaaatgctgaccctacagagacagtggtgcattttggccctggagaaagttcgaaagatgtcgtcatgatgagcacgcctgtggttaaagcagccttggaaatgggcttcagtaggagcctggtgagacagacggttcagcggcagatcctggccactggtgagaactacaggaccgtcaatgatattgtctcagtacttttgaatgctgaagatgagagaagagaagaggagaaggaaagacagactgaagagatggcatcaggtgacttatcactgattcggaagaatagaatggccctctttcaacagttgacacatgtccttcctatcctggataatcttcttgaggccagtgtaattacaaaacaggaacatgatattattagacagaaaacacagatacccttacaagcaagagagcttattgacaccgttttagtcaagggaaatgctgcagccaacatcttcaaaaactctctgaaggaaattgactccacgttatatgaaaacttatttgtggaaaagaatatgaagtatattccaacagaagacgtttcaggcttgtcattggaagagcagttgcggagattacaagaagaacgaacttgcaaagtgtgtatggacagagaggtttctattgtgttcattccgtgtggtcatctagtagtctgccaggaatgtgccccttctctaaggaagtgccccatctgcagggggacaatcaaggggactgtgcgcacatttctctcatgagtgaagaatggtctgaaagtattgttggacatcagaagctgtcagaacaaagaatgaactactgatttcagctcttcagcaggacattctactctctttcaagattagtaatcttgctttatgaagggtagcattgtatatttaagcttagtctgttgcaagggaaggtctatgctgttgagctacaggactgtgtctgttccagagcaggagttgggatgcttgctgtatgtccttcaggacttcttggatttggaatttgtgaaagctttggattcaggtgatgtggagctcagaaatcctgaaaccagtggctctggtactcagtagttagggtaccctgtgcttcttggtgcttttcctttctggaaaataaggatttttctgctactggtaaatattttctgtttgtgagaaatatattaaagtgtttcttttaaaggcgtgcatcattgtagtgtgtgcagggatgtatgcaggcaaaacactgtgtatataataaataaatctttttaaaaagtgtaaaaaaaaaaa

SEQ ID NO: 5 - protein, 496 aa, human
MTFNSFEGTRTFVLADTNKDEEFVEEFNRLKTFANFPSSSPVSASTLARAGFLYTGEGDTVQCFSCHAAIDRWQYGDSAVGRHRRISPNCRFINGFYFENGAAQSTNPGIQNGQYKSENCVGNRNPFAPDRPPETHADYLLRTGQVVDISDTIYPRNPAMCSEEARLKSFQNWPDYAHLTPRELASAGLYYTGADDQVQCFCCGGKLKNWEPCDRAWSEHRRHFPNCFFVLGRNVNVRSESGVSSDRNFPNSTNSPRNPAMAEYEARIVTFGTWTSSVNKEQLARAGFYALGEGDKVKCFHCGGGLTDWKPSEDPWDQHAKCYPGCKYLLDEKGQEYINNIHLTHPLEESLGRTAEKTPPLTKKIDDTIFQNPMVQEAIRMGFSFKDLKKTMEEKIQTSGSSYLSLEVLIADLVSAQKDNTEDESSQTSLQKDISTEEQLRRLQEEKLSKICMDRNIAIVFFPCGHLATCKQCAEAVDKCPMCYTVITFNQKIFMS

SEQ ID NO: 6 - DNA, 2691 bp, human
attttttaaattgatgcattaacattctaaacattcatctgtttttaaatagtaaaaattgaactttgccttgaatatgtaatgattcattataacaattatgcatagtctttaataatctgcatattttatgctgctttcatgtttttcctaattaatgacttcacatgtttaatatttataatttttctgtcatagtttccatatttatataaaatgaatacttaagatcagtaattctgctctgtttgtttatatactattttccatcaaaagacaaaatgggactgaggttgaggctcgttgctaaagcactttcctaaaatgcaaaaggccctatgatggatccctagtacttatttaagtgagagagaaacaggctgggggtgtaggtctgttagagcatgtgtttggcattatgtgaagcccaaacactaaaaaaggagaacaaacaaaagcgcagactttaaaactcaagtggtttggtaatgtacgactctactgtttagaattaaaatgtgtcttagttattgtgccattatttttatgtcatcactggataatatattagtgcttagtatcagaaatagtccttatgctttgtgttttgaagttcctaatgcaatgttctctttctagaaaaggtggacaagtcctattttccagagaagatgacttttaacagttttgaaggaactagaacttttgtacttgcagacaccaataaggatgaagaatttgtagaagagtttaatagattaaaaacatttgctaacttcccaagtagtagtcctgtttcagcatcaacattggcgcgagctgggtttctttataccggtgaaggagacaccgtgcaatgtttcagttgtcatgcggcaatagatagatggcagtatggagactcagctgttggaagacacaggagaatatccccaaattgcagatttatcaatggtttttattttgaaaatggtgctgcacagtctacaaatcctggtatccaaaatggccagtacaaatctgaaaactgtgtgggaaatagaaatccttttgcccctgacaggccacctgagactcatgctgattatctcttgagaactggacaggttgtagatatttcagacaccatatacccgaggaaccctgccatgtgtagtgaagaagccagattgaagtcatttcagaactggccggactatgctcatttaacccccagagagttagctagtgctggcctctactacacaggggctgatgatcaagtgcaatgcttttgttgtgggggaaaactgaaaaattgggaaccctgtgatcgtgcctggtcagaacacaggagacactttcccaattgcttttttgttttgggccggaacgttaatgttcgaagtgaatctggtgtgagttctgataggaatttcccaaattcaacaaactctccaagaaatccagccatggcagaatatgaagcacggatcgttacttttggaacatggacatcctcagttaacaaggagcagcttgcaagagctggattttatgctttaggtgaaggcgataaagtgaagtgcttccactgtggaggagggctcacggattggaagccaagtgaagacccctgggaccagcatgctaagtgctacccagggtgcaaatacctattggatgagaaggggcaagaatatataaataatattcatttaacccatccacttgaggaatctttgggaagaactgctgaaaaaacaccaccgctaactaaaaaaatcgatgataccatcttccagaatcctatggtgcaagaagctatacgaatgggatttagcttcaaggaccttaagaaaacaatggaagaaaaaatccaaacatccgggagcagctatctatcacttgaggtcctgattgcagatcttgtgagtgctcagaaagataatacggaggatgagtcaagtcaaacttcattgcagaaagacattagtactgaagagcagctaaggcgcctacaagaggagaagctttccaaaatctgtatggatagaaatattgctatcgttttttttccttgtggacatctggccacttgtaaacagtgtgcagaagcagttgacaaatgtcccatgtgctacaccgtcattacgttcaaccaaaaaatttttatgtcttagtggggcaccacatgttatgttcttcttgctctaattgaatgtgtaatgggagcgaactttaagtaatcctgcatttgcattccattagcatcctgctgtttccaaatggagaccaatgctaacagcactgtttccgtctaaacattcaatttctggatctttcgagttatcagctgtatcatttagccagtgttttactcgattgaaaccttagacagagaagcattttatagcttttcacatgtatattggtagtacactgacttgatttctatatgtaagtgaattcatcacctgcatgtttcatgccttttgcataagcttaacaaatggagtgttctgtataagcatggagatgtgatggaatctgcccaatgactttaattggcttattgtaaacacggaaagaactgccccacgctgctgggaggataaagattgttttagatgctcacttctgtgttttaggattctgcccatttacttggaatttattggagttataatgtacttatatgatatttccgaa

SEQ ID NO: 7 - protein, 459 aa, human
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASVICH

SEQ ID NO: 8 - DNA, 3495 bp, human
gaattctatggagtgtaattttgtgtatgaattatatttttaaaacattgaagagttttcagaaagaaggctagtagagttgattactgatactttatgctaagcagtacttttttggtagtacaatattttgttaggcgtttctgataacactagaaaggacaagttttatcttgtgataaattgattaatgtttacaacatgactgataattatagctgaatagtccttaaatgatgaacaggttatttagtttttaaatgcagtgtaaaaagtgtgctgtggaaattttatggctaactaagtttatggagaaaataccttcagttgatcaagaataatagtggtatacaaagttaggaagaaagtcaacatgatgctgcaggaaatggaaacaaatacaaatgatatttaacaaagatagagtttacagtttttgaactttaagccaaattcatttgacatcaagcactatagcaggcacaggttcaacaaagcttgtgggtattgacttcccccaaaagttgtcagctgaagtaatttagcccacttaagtaaatactatgatgataagctgtgtgaacttagcttttaaatagtgtgaccatatgaaggttttaattacttttgtttattggaataaaatgagattttttgggttgtcatgttaaagtgcttatagggaaagaagcctgcatataattttttaccttgtggcataatcagtaattggtctgttattcaggcttcatagcttgtaaccaaatataaataaaaggcataatttaggtattctatagttgcttagaattttgttaatataaatctctgtgaaaaatcaaggagttttaatattttcagaagtgcatccacctttcagggctttaagttagtattactcaagattatgaacaaatagcacttaggttacctgaaagagttactacaaccccaaagagttgtgttctaagtagtatcttggtaattcagagagatactcatcctacctgaatataaactgagataaatccagtaaagaaagtgtagtaaattctacataagagtctatcattgatttctttttgtggtaaaaatcttagttcatgtgaagaaatttcatgtgaatgttttagctatcaaacagtactgtcacctactcatgcacaaaactgcctcccaaagacttttcccaggtccctcgtatcaaaacattaagagtataatggaagatagcacgatcttgtcagattggacaaacagcaacaaacaaaaaatgaagtatgacttttcctgtgaactctacagaatgtctacatattcaactttccccgccggggtgcctgtctcagaaaggagtcttgctcgtgctggtttttattatactggtgtgaatgacaaggtcaaatgcttctgttgtggcctgatgctggataactggaaactaggagacagtcctattcaaaagcataaacagctatatcctagctgtagctttattcagaatctggtttcagctagtctgggatccacctctaagaatacgtctccaatgagaaacagttttgcacattcattatctcccaccttggaacatagtagcttgttcagtggttcttactccagcctttctccaaaccctcttaattctagagcagttgaagacatctcttcatcgaggactaacccctacagttatgcaatgagtactgaagaagccagatttcttacctaccatatgtggccattaacttttttgtcaccatcagaattggcaagagctggtttttattatataggacctggagatagggtagcctgctttgcctgtggtgggaagctcagtaactgggaaccaaaggatgatgctatgtcagaacaccggaggcattttcccaactgtccatttttggaaaattctctagaaactctgaggtttagcatttcaaatctgagcatgcagacacatgcagctcgaatgagaacatttatgtactggccatctagtgttccagttcagcctgagcagcttgcaagtgctggtttttattatgtgggtcgcaatgatgatgtcaaatgcttttgttgtgatggtggcttgaggtgttgggaatctggagatgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttcaaagtaaaatcctgacaactggagagaactataaaacagttaatgatattgtgtcagcacttcttaatgctgaagatgaaaaaagagaagaggagaaggaaaaacaagctgaagaaatggcatcagtgatttgtcattaattcggaagaacagaatggctctctttcaacaattgacatgtgtgcttcctatcctggataatcttttaaaggccaatgtaattaataaacaggaacatgatattattaaacaaaaaacacagatacctttacaagcgagagaactgattgataccattttggttaaaggaaatgctgcggccaacatcttcaaaaactgtctaaaagaaattgactctacattgtataagaacttatttgtggataagaatatgaagtatattccaacagaagatgtttcaggtctgtcactggaagaacaattgaggaggttgcaagaagaacgaacttgtaaagtgtgtatggacaaagaagtttctgttgtatttattccttgtggtcatctggtagtatgccaggaatgtgccccttctctaagaaaatgccctatttgcaggggtataatcaagggtactgttcgtacatttctctcttaaagaaaaatagtctatattttaacctgcataaaaaggtctttaaaatattgttgaacacttgaagccatctaaagtaaaaagggaattatgagtttttcaattagtaacattcatgttctagtctgctttggtactaataatcttgtttctgaaaagatggtatcatatatttaatcttaatctgtttatttacaagggaagatttatgtttggtgaactatattagtatgtatgtgtacctaagggagtagtgtcactgcttgttatgcatcatttcaggagttactggatttgttgttctttcagaaagctttgaatactaaattatagtgtagaaaagaactggaaaccaggaactctggagttcatcagagttatggtgccgaattgtctttggtgcttttcacttgtgttttaaaataaggatttttctcttatttctccccctagtttgtgagaaacatctcaataaagtgcttt

SEQ ID NO: 9 - protein, 557 aa, human
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSGSTSGRTIAETTRRKNM

SEQ ID NO: 10 - DNA, 3153 bp, human
gaattcaaaatgtcttcagttgtaaatcttaccattattttacgtacctctaagaaataaaagtgcttctaattaaaatatgatgtcattaattatgaaatacttcttgataacagaagttttaaaatagccatcttagaatcagtgaaatatggtaatgtattattttcctcctttgagttaggtcttgtgcttttttttcctggccactaaatttcacaatttccaaaaagcaaaataaacatattctgaatatttttgctgtgaaacacttgacagcagagctttccaccatgaaaagaagcttcatgagtcacacattacatctttgggttgattgaatgccactgaaacattctagtagcctggagaagttgacctacctgtggagatgcctgccattaaatggcatcctgatggcttaatacacatcactcttctgtgaagggttttaattttcaacacagcttactctgtagcatcatgtttacattgtatgtataaagattatacaaaggtgcaattgtgtatttcttccttaaaatgtatcagtataggatttagaatctccatgttgaaactctaaatgcatagaaataaaaataataaaaaatttttcattttggcttttcagcctagtattaaaactgataaaagcaaagccatgcacaaaactacctccctagagaaaggctagtcccttttcttccccattcatttcattatgaacatagtagaaaacagcatattcttatcaaatttgatgaaaagcgccaacacgtttgaactgaaatacgacttgtcatgtgaactgtaccgaatgtctacgtattccacttttcctgctggggttcctgtctcagaaaggagtcttgctcgtgctggtttctattacactggtgtgaatgacaaggtcaaatgcttctgttgtggcctgatgctggataactggaaaagaggagacagtcctactgaaaagcataaaaagttgtatcctagctgcagattcgttcagagtctaaattccgttaacaacttggaagctacctctcagcctacttttccttcttcagtaacaaattccacacactcattacttccgggtacagaaaacagtggatatttccgtggctcttattcaaactctccatcaaatcctgtaaactccagagcaaatcaagatttttctgccttgatgagaagttcctaccactgtgcaatgaataacgaaaatgccagattacttacttttcagacatggccattgacttttctgtcgccaacagatctggcaaaagcaggcttttactacataggacctggagacagagtggcttgctttgcctgtggtggaaaattgagcaattgggaaccgaaggataatgctatgtcagaacacctgagacattttcccaaatgcccatttatagaaaatcagcttcaagacacttcaagatacacagtttctaatctgagcatgcagacacatgcagcccgctttaaaacattctttaactggccctctagtgttctagttaatcctgagcagcttgcaagtgcgggtttttattatgtgggtaacagtgatgatgtcaaatgcttttgctgtgatggtggactcaggtgttgggaatctggagatgatccatgggttcaacatgccaagtggtttccaaggtgtgagtacttgataagaattaaaggacaggagttcatccgtcaagttcaagccagttaccctcatctacttgaacagctgctatccacatcagacagcccaggagatgaaaatgcagagtcatcaattatccattttgaacctggagaagaccattcagaagatgcaatcatgatgaatactcctgtgattaatgctgccgtggaaatgggctttagtagaagcctggtaaaacagacagttcagagaaaaatcctagcaactggagagaattatagactagtcaatgatcttgtgttagacttactcaatgcagaagatgaaataagggaagaggagagagaaagagcaactgaggaaaaagaatcaaatgatttattattaatccggaagaatagaatggcactttttcaacatttgacttgtgtaattccaatcctggatagtctactaactgccggaattattaatgaacaagaacatgatgttattaaacagaagacacagacgtctttacaagcaagagaactgattgatacgattttagtaaaaggaaatattgcagccactgtattcagaaactctctgcaagaagctgaagctgtgttatatgagcatttatttgtgcaacaggacataaaatatattcccacagaagatgtttcaggatctaccagtggaagaacaattgcggagactacaagaagaaagaacatgtaaagtgtgtatggacaaagaagtgtccatagtgtttattccttgtggtcatctagtagtatgcaaagattgtgctccttctttaagaaagtgtcctatttgtaggagtacaatcaagggtacagttcgtacatttctttcatgaagaagaaccaaaacatcgtctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaataccgggaacatgaagccaggtgtggtggtatgtgcctgtagtcccaggctgaggcaagagaattacttgagcccaggagtttgaatccatcctgggcagcatactgagaccctgcctttaaaaacaaacagaacaaaaacaaaacaccagggacacatttctctgtcttttttgatcagtgtcctatacatcgaaggtgtgcatatatgttgaatgacattttagggacatggtgtttttataaagaattctgtgagaaaaaatttaataaa

SEQ ID NO: 11 - protein, 618 aa, human
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 12 - DNA, 3495 bp, human
gaattctatggagtgtaattttgtgtatgaattatatttttaaaacattgaagagttttcagaaagaaggctagtagagttgattactgatactttatgctaagcagtacttttttggtagtacaatattttgttaggcgtttctgataacactagaaaggacaagttttatcttgtgataaattgattaatgtttacaacatgactgataattatagctgaatagtccttaaatgatgaacaggttatttagtttttaaatgcagtgtaaaaagtgtgctgtggaaattttatggctaactaagtttatggagaaaataccttcagttgatcaagaataatagtggtatacaaagttaggaagaaagtcaacatgatgctgcaggaaatggaaacaaatacaaatgatatttaacaaagatagagtttacagtttttgaactttaagccaaattcatttgacatcaagcactatagcaggcacaggttcaacaaagctttgggtattgacttcccccaaaagttgtcagctgaagtaatttagcccacttaagtaaatactatgatgataagctgtgtgaacttagcttttaaatagtgtgaccatatgaaggttttaattacttttgtttattggaataaaatgagattttttgggttgtcatgttaaagtgcttatagggaaagaagcctgcatataattttttaccttgtggcataatcagtaattggtctgttattcaggcttcatagcttgtaaccaaatataaataaaaggcataatttaggtattctatagttgcttagaattttgttaatataaatctctgtgaaaaatcaaggagttttaatattttcagaagtgcatccacctttcagggctttaagttagtattactcaagattatgaacaaatagcacttaggttacctgaaagagttactacaaccccaaagagttgtgttctaagtagtatcttggaaattcagagagatactcatcctacctgaatataaactgagataaatccagtaaagaaagtgtagtaaattctacataagagtctatcattgatttcttttggtggtaaaaatcttagttcatgtgaagaaatttcatgtgaatgttttagctatcaaacagcactgtcacctactcatgcacaaaactgcctcccaaagacttttcccaggtccctcgtatcaaaacattaagagtataatggaagatagcacgatcttgtcagattggacaaacagcaacaaacaaaaaatgaagtatgacttttcctgtgaactctacagaatgtctacatattcaactttccccgccggggtgcctgtctcagaaaggagtcttgctcgtgctggtttttattatactggtgtgaatgacaaggtcaaatgcttctgttgtggcctgatgctggataactggaaactaggagacagtcctattcaaaagcataaacagctatatcctagctgtagctttattcagaatctggtttcagctagtctgggatccacctctaagaatacgtctccaatgagaaacagttttgcacattcattatctcccaccttggaacatagtagcttgttcagtggttcttactccagcctttctccaaaccctcttaattctagagcagttgaagacatctcttcatcgaggactaacccctacagttatgcaatgagtactgaagaagccagatttcttacctaccatatgtggccattaacttttttgtcaccatcagaattggcaagagctggtttttattatataggacctggagatagggtagcctgctttgcctgtggtgggaagctcagtaactgggaaccaaaggatgatgctatgtcagaacaccggaggcattttcccaactgtccatttttggaaaattctctagaaactctgaggtttagcatttcaaatctgagcatgcagacacatgcagctcgaatgagaacatttatgtactggccatctagtgttccagttcagcctgagcagcttgcaagtgctggtttttattatgtgggtcgcaatgatgatgtcaaatgcttttgttgtgatggtggcttgaggtgttgggaatctggagatgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttcaaagtaaaatcctgacaactggagagaactataaaacagttaatgatattgtgtcagcacttctaaatgctgaagatgaaaaaagagaggaggagaaggaaaaacaagctgaagaaatggcatcagatgatttgtcattaattcggaagaacagaatggctctctttcaacaattgacatgtgtgcttcctatcctggataatcttttaaaggccaatgtaattaataaacaggaacatgatattattaaacaaaaaacacagatacctttacaagcgagagaactgattgataccattttggttaaaggaaatgctgcggccaacatcttcaaaaactgtctaaaagaaattgactctacattgtataagaacttatttgtggataagaatatgaagtatattccaacagaagatgtttcaggtctgtcactggaagaacaattgaggaggttgcaagaagaacgaacttgtaaagtgtgtatggacaaagaagtttctgttgtatttattccttgtggtcatctggtagtatgccaggaatgtgccccttctctaagaaaatgccctatttgcaggggtataatcaagggtactgttcgtacatttctctcttaaagaaaaatagtctatattttaacctgcataaaaaggtctttaaaatattgttgaacacttgaagccatctaaagtaaaaagggaattatgagtttttcaattagtaacattcatgttctagtctgctttggtactaataatcttgtttctgaaaagatggtatcatatatttaatcttaatctgtttatttacaagggaagatttatgtttggtgaactatattagtatgtatgtgtacctaagggagtagtgtcactgcttgttatgcatcatttcaggagttactggatttgttgttctttcagaaagctttgaatactaaattatagtgtagaaaagaactggaaaccaggaactctggagttcatcagagttatggtgccgaattgtctttggtgcttttcacttgtgttttaaaataaggatttttctcttatttctccccctagtttgtgagaaacatctcaataaagtgcttt

SEQ ID NO: 13 - protein, 806 aa, human
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 14 - protein, 142 aa, human
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 15 - DNA, 1662 bp, human
ggcacgagggcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaaaaaaaaaaaaaaaaaaaaaaaaaaaaaaaaaaaaaaaaaaaaaaaa

SEQ ID NO: 16 - protein, 1248 aa, human
MDAKARNCLLQHREALEKDIKTSYIMDHMISDGFLTISEEEKVRNEPTQQQRAAMLIKMILKKDNDSYVSFYNALLHEGYKDLAALLHDGIPVVSSSSGKDSVSGITSYVRTVLCEGGVPQRPVVFVTRKKLVNAIQQKLSKLKGEPGWVTIHGMAGCGKSVLAAEAVRDHSLLEGCFPGGVHWVSVGKQDKSGLLMKLQNLCTRLDQDESFSQRLPLNIEEAKDRLRILMLRKHPRSLLILDDVWDSWVLKAFDSQCQILLTTRDKSVTDSVMGPKYVVPVESSLGKEKGLEILSLFVNMKKADLPEQAHSIIKECKGSPLVVSLIGALLRDFPNRWEYYLKQLQNKQFKRIRKSSSYDYEALDEAMSISVEMLREDIKDYYTDLSILQKDVKVPTKVLCILWDMETEEVEDILQEFVNKSLLFCDRNGKSFRYYLHDLQVDFLTEKNCSQLQDLHKKIITQFQRYHQPHTLSPDQEDCMYWYNFLAYHMASAKMHKELCALMFSLDWIKAKTELVGPAHLIHEFVEYRHILDEKDCAVSENFQEFLSLNGHLLGRQPFPNIVQLGLCEPETSEVYQQAKLQAKQEVDNGMLYLEWINKKNITNLSRLVVRPHTDAVYHACFSEDGQRIASCGADKTLQVFKAETGEKLLEIKAHEDEVLCCAFSTDDRFIATCSVDKKVKIWNSMTGELVHTYDEHSEQVNCCHFTNSSHHLLLATGSSDCFLKLWDLNQKECRNTMFGHTNSVNHCRFSPDDKLLASCSADGTLKLWDATSANERKSINVKQFFLNLEDPQEDMEVIVKCCSWSADGARIMVAAKNKIFLFDIHTSGLLGEIHTGHHSTIQYCDFSPQNHLAVVALSQYCVELWNTDSRSKVADCRGHLSWVHGVMFSPDGSSFLTSSDDQTIRLWETKKVCKNSAVMLKQEVDVVFQENEVMVLAVDHIRRLQLINGRTGQIDYLTEAQVSCCCLSPHLQYIAFGDENGAIEILELVNNRIFQSRFQHKKTVWHIQFTADEKTLISSSDDAEIQVWNWQLDKCIFLRGHQETVKDFRLLKNSRLLSWSFDGTVKVWNIITGNKEKDFVCHQGTVLSCDISHDATKFSSTSADKTAKIWSFDLLLPLHELRGHNGCVRCSAFSVDSTLLATGDDNGEIRIWNVSNGELLHLCAPLSEEGAATHGGWVTDLCFSPDGKMLISAGGYIKWWNVVTGESSQTFYTNGTNLKKIHVSPDFKTYVTVDNLGILYILQTLE

SEQ ID NO: 17 - DNA, 3747 bp, human
atggatgcaaaagctcgaaattgtttgcttcaacatagagaagctctggaaaaggacatcaagacatcctacatcatggatcacatgattagtgatggatttttaacaatatcagaagaggaaaaagtaagaaatgagcccactcaacagcaaagagcagctatgctgattaaaatgatacttaaaaaagataatgattcctacgtatcattctacaatgctctactacatgaaggatataaagatcttgctgcccttctccatgatggcattcctgttgtctcttcttccagtggtaaagattcagttagtggaataacttcgtatgtaaggacagtcctgtgtgaaggtggagtaccacagaggccagttgtttttgtcacaaggaagaagctggtgaatgcaattcagcagaagctctccaaattgaaaggtgaaccaggatgggtcaccatacatggaatggcaggctgtgggaagtctgtattagctgcagaagctgttagagatcattcccttttagaaggttgtttcccagggggagtgcattgggtttcagttgggaaacaagacaaatctgggcttctgatgaaactgcagaatctttgcacacggttggatcaggatgagagtttttcccagaggcttccacttaatattgaagaggctaaagaccgtctccgcattctgatgcttcgcaaacacccaaggtctctcttgatcttggatgatgtttgggactcttgggtgttgaaagcttttgacagtcagtgtcagattcttcttacaaccagagacaagagtgttacagattcagtaatgggtcctaaatatgtagtccctgtggagagttccttaggaaaggaaaaaggacttgaaattttatccctttttgttaatatgaagaaggcagatttgccagaacaagctcatagtattataaaagaatgtaaaggctctccccttgtagtatctttaattggtgcacttttacgtgattttcccaatcgctgggagtactacctcaaacagcttcagaataagcagtttaagagaataaggaaatcttcgtcttatgattatgaggctctagatgaagccatgtctataagtgttgaaatgctcagagaagacatcaaagattattacacagatctttccatccttcagaaggacgttaaggtgcctacaaaggtgttatgtattctctgggacatggaaactgaagaagttgaagacatactgcaggagtttgtaaataagtctcttttattctgtgatcggaatggaaagtcgtttcgttattatttacatgatcttcaagtagattttcttacagagaagaattgcagccagcttcaggatctacataagaagataatcactcagtttcagagatatcaccagccgcatactctttcaccagatcaggaagactgtatgtattggtacaactttctggcctatcacatggccagtgccaagatgcacaaggaactttgtgctttaatgttttccctggattggattaaagcaaaaacagaacttgtaggccctgctcatctgattcatgaatttgtggaatacagacatatactagatgaaaaggattgtgcagtcagtgagaattttcaggagtttttatctttaaatggacaccttcttggacgacagccatttcctaatattgtacaactgggtctctgtgagccggaaacttcagaagtttatcagcaagctaagctgcaggccaagcaggaggtcgataatggaatgctttacctggaatggataaacaaaaaaaacatcacgaatctttcccgcttagttgtccgcccccacacagatgctgtttaccatgcctgcttttctgaggatggtcagagaatagcttcttgtggagctgataaaaccttacaggtgttcaaagctgaaacaggagagaaacttctagaaatcaaggctcatgaggatgaagtgctttgttgtgcattctctacagatgacagatttatagcaacctgctcagtggataaaaaagtgaagatttggaattctatgactggggaactagtacacacctatgatgagcactcagagcaagtcaattgctgccatttcaccaacagtagtcatcatcttctcttagccactgggtcaagtgactgcttcctcaaactttgggatttgaatcaaaaagaatgtcgaaataccatgtttggtcatacaaattcagtcaatcactgcagattttcaccagatgataagcttttggctagttgttcagctgatggaaccttaaagctttgggatgcgacatcagcaaatgagaggaaaagcattaatgtgaaacagttcttcctaaatttggaggaccctcaagaggatatggaagtgatagtgaagtgttgttcgtggtctgctgatggtgcaaggataatggtggcagcaaaaaataaaatctttctttttgacattcatactagtggcctattgggagaaatccacacgggccatcacagcaccatccagtactgtgacttctccccacaaaaccatttggcagtggttgctttgtcccagtactgtgtagagttgtggaatacagactcacgttcaaaggtggctgattgcagaggacatttaagttgggttcatggtgtgatgttttctcctgatggatcatcatttttgacatcttctgatgaccagacaatcaggctctgggagacaaagaaagtatgtaagaactctgctgtaatgttaaagcaagaagtagatgttgtgtttcaagaaaatgaagtgatggtccttgcagttgaccatataagacgtctgcaactcattaatggaagaacaggtcagattgattatctgactgaagctcaagttagctgctgttgcttaagtccacatcttcagtacattgcatttggagatgaaaatggagccattgagattttagaacttgtaaacaatagaatcttccagtccaggtttcagcacaagaaaactgtatggcacatccagttcacagccgatgagaagactcttatttcaagttctgatgatgctgaaattcaggtatggaattggcaattggacaaatgtatctttctacgaggccatcaggaaacagtgaaagactttagactcttgaaaaattcaagactgctttcttggtcatttgatggaacagtgaaggtatggaatattattactggaaataaagaaaaagactttgtctgtcaccagggtacagtactttcttgtgacatttctcacgatgctaccaagttttcatctacctctgctgacaagactgcaaagatctggagttttgatctccttttgccacttcatgaattgaggggccacaacggctgtgtgcgctgctctgccttctctgtggacagtaccctgctggcaacgggagatgacaatggagaaatcaggatatggaatgtctcaaacggtgagcttcttcatttgtgtgctccgctttcagaagaaggagctgctacccatggaggctgggtgactgacctttgcttttctccagatggcaaaatgcttatctctgctggaggatatattaagtggtggaacgttgtcactggggaatcctcacagaccttctacacaaatggaaccaatcttaagaaaatacacgtgtcccctgacttcaaaacatatgtgactgtggataatcttggtattttatatattttacagactttagaataa

SEQ ID NO: 18 - protein, 199 aa, human [sequence omitted]

SEQ ID NO: 19 - DNA, 600 bp, human
atggaagccagagacaagcaggtactccgctccctgcgtctggagctgggtgccgaggtactggtggaaggactggttcttcagtacctttaccaggaaggaattttgacagaaaaccacattcaagaaatcaaagctcaaaccacaggcctccggaagacaatgctgttgctggacatcctgccttccaggggccccaaagcttttgacaccttcctcgattccctccaggaatttccctgggtaagagagaagctggagaaggcgagagaggaagtcacagccgagctgcctacaggtgactggatggccggaatcccctcacacatcctcagcagctcgccatcagaccagcagattaaccagctggctcagaagctaggcccggagtgggagcccgtggtcctgtctctgggactgtcccagaccgacatctaccgctgcaaggccaaccatccccacaacgtgcattcgcaggtggtggaggcctttgtccgctggcgccagcgttttgggaagcaggccaccttcctaagcttacacaagggcctccaggcagtggaggctgatccctccctgctccagcacatgctggagtga

SEQ ID NO: 20 - protein, 239 aa, human
MAALKSWLSRSLTSTSRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 21 - DNA, 1351 bp, human
ggcgtccgcgcgctgcacaatggcggctctgaagagttggctgtcgcgcagcctgacttctacttccaggtacagacagtgtttgtgtgttcctgttgtggctaactttaagaagcggtgtttctcagaattgataagaccatggcacaaaactgtgacgattggctttggagtaaccctgtgtgcggttcctattgcacagaaatcagagcctcattcccttagtagtgaagcattgatgaggagagcagtgtctttggtaacagatagcacctctacctttctctctcagaccacatatgcgttgattgaagctattactgaatatactaaggctgtttataccttaacttctctttaccgacaatatacaagtttacttgggaaaatgaattcagaggaggaagatgaagtgtggcaggtgatcataggagccagagctgagatgacttcaaaacaccaagagtacttgaagctggaaaccacttggatgactgcagttggtctttcagagatggcagcagaagctgcatatcaaactggcgcagatcaggcctctataaccgccaggaatcacattcagctggtgaaactgcaggtggaagaggtgcaccagctctcccggaaagcagaaaccaagctggcagaagcacagatagaagagctccgtcagaaaacacaggaggaaggggaggagcgggctgagtcggagcaggaggcctacctgcgtgaggattgagggcctgagcacactgccctgtctccccactcagtggggaaagcaggggcagatgccaccctgcccagggttggcatgactgtctgtgcaccgagaagaggcggcagatcctgccctggccaatcaggcgagacgcctttgtgagctgtgagtgcctcctgtggtctcaggcttgcgctggacctggttcttagcccttgggcactgcaccctgtttaacatttcaccccactctgtacagctgctcttacccattttttttacctcacacccaaagcattttgcctacctgggtcagagagaggagtcctttttgtcatgcccttaagttcagcaactgtttaacctgttttcagtcttatttacgtcgtcaaaaatgatttagtacttgttccctctgttgggatgccagttgtggcagggggaggggaacctgtccagtttgtacgatttctttgtatgtatttctgatgtgttctctgatctgcccccactgtcctgtgaggacagctgaggccaaggagtgaaaaacctattactactaagagaaggggtgcagagtgtttacctggtgctctcaacaggacttaacatcaacaggacttaacacag

SEQ ID NO: 22 - protein, 1403 aa, human
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK